(19) United States
(12) Patent Application Publication
(10) Pub. No.: US 2026/0234698 A1
(43) Pub. Date: Aug. 13, 2026

(54) DEVICES AND METHODS FOR MACROMOLECULAR MANIPULATION

(71) Applicants: DIMENSIONGEN, GRAND CAYMAN (KY); Dimension Genomics Inc, San Diego, CA (US)

(72) Inventors: Michael David Austin, San Diego, CA (US); Han Cao, San Diego, CA (US)

(73) Assignees: DIMENSIONGEN, GRAND CAYMAN (KY); Dimension Genomics Inc, San Diego, CA (US)

(21) Appl. No.: 19/426,152

(22) Filed: Dec. 19, 2025

Related U.S. Application Data
(63) Continuation of application No. 17/921,219, filed on Apr. 29, 2021, now abandoned.
(60) Provisional application No. 63/017,650, filed on Apr. 30, 2020.
(60) Provisional application No. 63/087,131, filed on Oct. 2, 2020.
(60) Provisional application No. 63/143,857, filed on Jan. 31, 2021.

(51) Int. Cl. C12Q 1/6806; C12Q 1/6816

(52) U.S. Cl. CPC C12Q 1/6806; C12Q 1/6816

(57) ABSTRACT

Disclosed herein are methods, compositions and systems for the interrogation of macromolecules, more particularly for preparation of isolated single macromolecules for subsequent processing of specific regions of interest within said macromolecule based on an analysis of the molecule's physical map. The disclosure is further related to the controlled segmentation of long nucleic acid parent molecules into smaller child molecules in a targeted manner such that further processing on said children may be performed with the knowledge of their origin within the parent, in a controlled environment enabled by purposefully designed microfluidic devices. Also disclosed is binding of regional specific barcodes along the length of a long nucleic acid molecule such that upon cleavage of said molecule into child molecules, the regional origin of the children can be tracked, in a controlled environment enabled by purposefully designed microfluidic devices. Finally, the disclosure is further related to droplet devices and method to control the encapsulation of long nucleic acid molecules or specific subregions thereof into a droplet, and further tracking said droplets with their contents.

### CROSS-REFERENCE TO RELATED APPLICATIONS

This document claims the benefit of priority to U.S. Provisional Application Ser. No. 63/017,650, filed Apr. 30, 2020, to U.S. Provisional Application Ser. No. 63/087,131, filed Oct. 2, 2020, and to U.S. Provisional Application Ser. No. 63/143,857, filed Jan. 31, 2021, each of which is hereby incorporated by reference in its entirety.

### BACKGROUND

It is now widely known that genomic materials including chromosomes, extrachromosomal DNA, exogenous and transcribed RNAs are distinct and heterogenous among cells from tissues of the same individual, such as in the cases of de novo mutations, mosaicism, cancer, or neuron development. Furthermore, they can change dynamically within the same cell along the natural course of time, for example stimulated from a pathological development such as an infectious event or mutation, or in divergent environment with external stimulus. Ideally, genomic and proteomic analysis technology should be able to detect and discern these differences and changes at individual cellular and subcellular level with structural, environmental, spatial, and chronological context.

A chromosome is a deoxyribonucleic acid (DNA) molecule that contains all or part of the genetic material of an organism, its “genome”. Most eukaryotic chromosomes include packaging proteins which, aided by chaperone proteins, bind to and condense the DNA molecule to prevent it from becoming an unmanageable tangle [Hammond, 2017][Wilson, 2002]. For example, an average freely suspended human cell in solution with diameter of 20-100 um (diploid) contains about 6.4 billion base pairs of DNA divided among 46 chromosomes. The length of each base pair is about 0.34 nm. Therefore, if the DNA molecule in a diploid cell were elongated, and laid out end to end, the total length of DNA would be approximately 2 meters, and yet remarkably this genomic material can fit in a cell nucleus of diameter 10 micrometers in an organized manner. This is accomplished by packaging the DNA in cells in highly ordered three-dimensional chromosomes. Furthermore, the genome structure of this packaging plays a significant functional role in gene transcriptional regulation [Bonev, 2016]. Chromosomes are normally visible under a light microscope only when the cell is undergoing the metaphase of cell division (where all chromosomes are aligned in the center of the cell in their condensed form) [Alberts, 2014]. Before this happens, every chromosome is copied once (S phase), and the copy is joined to the original by a centromere, resulting either in an X-shaped structure if the centromere is located in the middle of the chromosome, or a two-arm structure if the centromere is located near one of the ends. The original chromosome and the copy are now called sister chromatids. During anaphase and metaphase, chromosomes in highly condensed discrete particle-like form, are easiest to distinguish and study for genetic abnormalities [Schleyden, 1847] [Antonin, 2016]. In human cells, the typical metaphase chromosome size has an approximate dimension of 1.4 micron in width to 10 microns in length. Chromosomal recombination during meiosis and subsequent sexual reproduction plays a significant role in genetic diversity. These genomic content and structures could be subject to many known and unknown factors, through processes known as chromosomal instability, can result in changes ranging from simpler rearrangements such as inversion, translocation, to highly complex chromoanagenesis [Pihan, 2013] such as chromoplexy [Shen, 2013] and chromothripsis [Maher, 2012] [Stephens, 2011]. Usually, this will make the cell initiate apoptosis leading to its own death, but sometimes mutations in the cell hamper this process and thus cause progression of cancer or developmental and congenital disorders.

Extrachromosomal DNA (abbreviated ecDNA) is any DNA that is found off the chromosomes, either inside or outside the nucleus of a cell, serving important biological functions [Rush, 1985] and playing a role in disease, such as ecDNA in cancer [Verhaak, 2019]. Besides plasmids, Mitochondrial and viral DNA, nuclear ecDNA molecules in tumor cells are considered to be a primary mechanism of gene amplification, resulting in many copies of driver oncogenes and very aggressive cancers [Nathanson, 2014][deCarvalho, 2018][Turner, 2017].

Cytogenetics is the study of chromosomes, which are long strands of DNA and protein that contain most of the genetic information in a cell. Cytogenetics involves testing samples of tissue, blood, Amniotic fluid or bone marrow in a laboratory to look for changes in chromosomes, including broken, missing, rearranged, or extra chromosomes. Changes in certain chromosomes may be a sign of a genetic disease or condition or some types of cancer. Cytogenetics may be used to help diagnose a disease or condition, plan treatment, or find out how well treatment is working. Techniques used include karyotyping, analysis of G-banded chromosomes, other cytogenetic optical banding techniques, as well as molecular cytogenetics such as fluorescent in situ hybridization (FISH) and comparative genomic hybridization (CGH). For example, the Mitelman Database of Chromosome Aberrations and Gene Fusions in Cancer is just one of the databases, supported by National Cancer Institute (NCI), has catalogued a total number of published unique clinical cases of 70,469 (July 2020), with a total number of unique gene fusions of 32,551 and a total number of genes involved of 14,014, since they started to collect information in 1983 (3844 cases) [Mitelman, 2020]. These chromosome aberrations were largely discovered by the gold standards of first-line clinical cytogenetics testing (karyotyping, FISH, Array, and CGH), with guidelines recommended by American College of Medical Genetics (ACMG), American Society for Clinical Pathology (ASCP), National Comprehensive Cancer Network (NCCN), American College of Medical Genetics (ACMG), American College of Obstetricians/Gynecologists (ACOG), and World Health Organization (WHO).

Genome Analysis via sequencing has rapidly expanded the advancement of next generation sequencing (NGS) technologies. Using such technologies, a large data base of genomic changes mostly comprised with millions of point mutations and SNPs (Single Nucleotide Polymorphisms) in human or biological populations have been generated, but direct clinical utility of the vast majority of these genomic changes remains unproven. There are over 7,000 known genetic disorders yet more than ⅔ remain without a clear understanding of the genetic cause, with some take 5-7 years to reach a diagnosis [Mayo website]. Despite the progress of NGS technology, the cost and technology limitations (for example limited native read length, library bias, and the bioinformatics complexity, to name a few) have prevented NGS's wider adoption into more clinical settings.

NGS provides a gain in genome nucleotide resolution, but at the expense of a loss in spatial and structural resolution of the chromosomes and genome analysis. In addition, with a large portion of the genome comprising “Dark Matter” (highly repetitive and variable regions difficult for sequencing and computation) that is still not easily accessible, NGS technologies have yet to provide true diploid/multiploidy medical grade genome data that is critical for a clinical environment. In addition, complete extrachromosomal DNA (ecDNA) information and complex chromothripsis structures remains elusive, as NGS sample prep and algorithms cannot distinguish them a priori. NGS data for accurately identifying structural variants is largely limited to SNPs and short indels.

The ability to efficiently sequence small quantities of nucleic acid, e.g., DNA, is important for applications ranging from the assembly of uncultivable microbial genomes to the identification of cancer associated mutations. To obtain enough nucleic acid for sequencing, the limited starting material must be amplified significantly. Recently, sample prep technologies to tailor sequencing methods towards single cell level have emerged, at least targeting the expressed portion of the genome, such as mRNAs. Single cell sequencing is an invaluable tool in microbial ecology and has enhanced the analysis of communities ranging from the ocean [Yoon, 2011] to the human mouth [Marcy, 2007]. Because majority of microorganisms cannot be cultured [Hutchison, 2006], obtaining sufficient quantity of DNA for sequencing requires significant amplification of single-cell genomes. However, existing methods are prone to amplification bias, often yield errors or non-uniformity of coverage, making sequencing inefficient and costly. Consequently, there has been a sustained effort to develop new methods to uniformly amplify small quantities of DNA.

One method is to modify the PCR reaction to enable non-specific amplification. Primer Extension Preamplification (PEP) and Degenerate Oligonucleotide-Primed PCR (DOP-PCR), for example, use modified primers and thermal cycling conditions to enable non-specific annealing and amplification of most DNA sequences [Zhang, 1992, Telenius, 1992]. However, amplification bias remains a major challenge for these methods: the products typically do not fully cover the original template and possess significant variation in coverage [Dean, 2002]. Multiple Annealing and Looping Based Amplification Cycles (MALBAC) reduces this bias with primers that cause amplicons to self-anneal in a loop; this suppresses exponential amplification of dominant products and equalizes amplification across the templates [Zong, 2012]. Nevertheless, the specialized polymerase required for this reaction is prone to copy errors that propagate through cycling, resulting in increased error rates. Multiple displacement amplification (MDA) enables non-specific amplification with minimal error through the use of the highly accurate enzyme Φ29 DNA polymerase [Esteban, 1993]. In addition, Φ29 DNA polymerase displaces Watson-Crick base-paired strands, enabling exponential amplification of template molecules without thermally induced denaturation [Dean, 2002]. Nevertheless, two major problems persist with MDA: amplification of contaminating DNA [Raghunathan, 2005] and highly uneven amplification of single-cell genomes [Dean, 2001] [Hosono, 2003]. These problems yield numerous challenges when sequencing MDA-amplified material, including incomplete genome assembly, gaps in genome coverage, and biased counts of replicated sequences, which are of biological relevance in a variety of applications such as assessing copy number variants in cancer. Due to its simplicity and accuracy, several strategies have been employed to reduce MDA amplification bias, including augmenting reactions with trehalose [Pan, 2008], reducing reaction volumes [Hutchison, 2005], and using nanoliter-scale microfluidic chambers to reduce the diversity in isolated pools [Marcy, 2007], [Gole, 2013, 2016/0138013]. While these methods help to mitigate the problems associated with MDA, robust and uniform amplification of low-input material remains a challenge.

Targeted DNA capture for sequencing comes in two main forms, amplicon or capture-based. Amplicon-based enrichment utilizes specifically designed primers to amplify only the regions of interest prior to library preparation [Samorodnitsky, 2015]. Alternatively, in capture-based approaches, the DNA is fragmented and targeted regions are enriched via hybridization oligonucleotide bait sequences attached to biotinylated probes, allowing for isolation from the remaining genetic material [Samorodnitsky, 2015, Mertes, 2011]. Amplicon-based enrichment is the cheaper of the two technologies and shows a greater number of on target reads; however, the coverage of these regions is more uniform with hybrid sequencing [Samorodnitsky, 2015, Hung, 2018]. Some commercially available amplicon platforms attempt to address the coverage issues by using specific primers that can amplify overlapping fragments in a single PCR reaction [Schenk, 2017]. Amplicon based sequencing requires much less starting material than hybrid-capture, making it ideal if there is little DNA available. Hybrid-capture has been shown to produce fewer PCR duplicates than amplicon enrichment (<40% and up to ~80%, respectively) [Samorodnitsky, 2015]. These duplicates are also more trivial to remove computationally, as the random shearing of the DNA in hybrid-capture platforms reduces the likelihood of two unique fragments aligning to the same genomic coordinates compared with the identical amplicons generated by amplicon enrichment platforms. This makes hybrid-capture especially useful for samples where these PCR artefacts are more likely to occur, such as FFPE and ctDNA samples. Further, certain regions of the genome make primer design for amplicon enrichment difficult (e.g. regions with a high number of repeated sequences). The long bait sequences used in hybrid-capture, however, allow a greater level of specificity in region selection. Overall hybrid-capture based platforms provide more accurate and uniform target selection, whilst amplicon-based platforms are often used in small scale experiments where sample quantity or cost are a factor.

For all targeted capture technologies, the capture mechanism is based on hybridization of a specific probe to a specific target, and thus, knowledge a priori of the desired target at nucleotide level to be captured. However, in some applications, the desired target may not match the probe due to mutations, or the desired target may not be based on a specific sequence, but a more complex requirement based on context of the genome in which the target lies. For example, distance from a known gene, or a known or unknown structural variant or features of interests suspected in a disease etiology.

The well established cytogenetics techniques of optically interrogating chromosome spreads are comprised of protocols and tools used by clinicians in tens of thousands hospital and clinical labs [Gersen, 2013]. They represent an elegant “top down” true single-cell, single-molecule level test with full real time optical view of the entire genomic chromosomal sets. The techniques remain the gold standard of first-line tests with well-established protocols and guidelines, with results trusted by doctors and clinicians. However, despite the historical success of these techniques, significant technological challenges have limited their potential to lower cost, improve quality, reduce errors, improve resolution of genomic changes, and most importantly high scalability. These limitations include: Labor-intensive procedures requiring subjective manual involvement of highly trained professionals (100~500 tests/person/year) [NPAAC, 2013], low resolution of identifying genomic changes, limited to megabases or larger, long turn-around time from sample to answer (typically 3~28 days), and ambiguous or erroneous raw image datasets that hinder the adaption of machine learning and require manual curation.

Clinical Samples are extremely complex, individualized and heterogeneous, at cellular and molecular levels. Large amounts of chromosomal lesions and rearrangements are well known. Large structural or numerical aberrations affect biological functions and are associated with complex diseases such as developmental and mental disorders, rare & undiagnosed diseases, reproductive anomalies, blood and all cancers. Technologies based on bottom-up ensemble data averaging using mixtures of cells and molecules address some questions in germline domain but come short in more challenging heterogeneous and dynamically complex clinical samples, in de novo, somatic, real time or early diagnostic settings. Today, most patients' tests are done with bulk solution assay using mixtures of cell/molecule samples, on different technology platforms, samples drawn and prepared at different time with different workflows, data interpreted in different labs with different algorithms on essentially different cells/molecules of even the same patient sample, averaged ensemble data report often yields inconclusive or even misleading results of subpar sensitivity and specificity. Often clinically significant biomarker signal arises in the earliest stages of a disease progression such as cancer that could be ‘drown-out” by ensemble measurement indicating a “normal” outcome; using less sensitive and less specific ensemble technology, a detectable averaged value based on large number of cells or molecules often only yield meaningful diagnostic conclusion when a consensus call is achieved, and usually during the late stage of the cancer when the best therapeutic intervention window has passed. Next generation single cell, subnuclear and single molecule level technologies, with capability to follow the exact same clinical sample by precisely compartmentalizing specific target cell, chromosome, molecules or subsegment of a molecule, while extracting multiple layers of diagnostic values in targeted regions of interests on the very same clinically relevant specific analytes in standardized workflow, are urgently needed. New technologies that overcome those current limitations, while maintaining the clinical validity of traditional single cell single molecule level cytogenetic methods, will allow for improved patient and doctor access to the most accurate and actionable genomic information of clinical value and help fulfill the promise of individualized medicine.

### SUMMARY OF THE INVENTION

The present disclosure provides devices and methods that facilitate the preparation of single long nucleic acid molecules for further processing or analysis.

In one set of embodiments, the disclosed devices and methods allow for the preparation of at least one ROI (region of interest) contained within a long genomic molecule, identified by the interrogation and analysis of a physical map on said molecule while in a fluidic device. The identification of the ROI by the interrogation and analysis of the physical map, followed by the ability of the device to arbitrarily target any number of ROI(s) of a certain size range along the length of the molecules allows for very flexible possibilities as to what can constitute an ROL Here, the ROI selection is not based on specificity of binding partners that must be per-determined, but instead can be assigned on-the-fly based on requirements that can change with time, or user preferences. The ROI may be a gene, a structural variation (SV), a methylation pattern, a labelling body, a physical map region. The ROI may be an unidentified region within the physical map, or a region that may have an association with another ROL directly or indirectly. The ROI may be a regulatory region, or a transcription factor binding site. The ROI may be a chromosomal region, a chromatin section, a compaction feature, an interaction or binding site, a regulatory factor or complex, a binding site, a transcription factor binding site, a TAD, a CRISPR binding site or complex, an SV, a phasing block, a regulatory or modification enzyme binding site, a restriction enzymes sequence motif, a methylation binding body, a centromeric region, a sub-telomeric region, a portion of telomere, a mobile element, a repetitive element, a viral insertion site. The ROI may be selected by some computer algorithm, or patent diagnosis, or discase hypothesis, or experimental hypothesis. The ROI may be selected by the user on-the-fly, or selected based on observations and analysis of other ROIs. The ROI may be selected based on the analysis of physical maps of other long nucleic acid molecules. To facilitate this set of embodiments, we disclose a variety of different device and method embodiments that allow either for the physical isolation of the ROI(s) from the parent molecule such that they can be processed independently, or for the targeted exposure of reagents, photons, or contact probes to the ROI(s) while still part of the parent molecule.

The ability to selectively expose ROI(s) along a nucleic acid molecule to reagents, photons, or a contact probe, while maintaining the integrity of the entire fragment, has many potential applications: motif labeling optical mapping, binding primers to enable local amplification, localized cleaving, localized enzymatic or binding events, cytogenetic G banding, gene editing/therapy, to name a few. Nucleic acid molecule interaction with biological entities typically is initiated with a random process of the entity encountering the nucleic acid molecule in a liquid bulk solution assay environment. This may be inefficient depending on the entity's concentration in the solution, furthermore there is no way of controlling which portion of the nucleic acid molecule is exposed to said reagents, and thus may result in undesired exposure to other regions of the nucleic acid molecule. Addressing the issue of undesired exposure via cleaving (physical separation) of the nucleic acid segments of interest results in fragmentation of the original nucleic acid molecule, which may have negative impact on downstream applications where nucleic acid molecule integrity is valuable.

In some embodiments, the ROI(s) are bound to universal primers such that the ROI(s) can be specifically amplified from the parent molecule, either at the time of binding, or at some later date, potentially on a different device. In some embodiments, the parent molecule is randomly bound to universal primers that are non-active, and then the primers in the ROI(s) are selectively photo-activated. In some embodiments, the parent molecule is randomly exposed to captured universal primers that cannot hybridize, then the captured universal primers are selectively photo-released in the area around the ROI(s), enabling the primers to hybridize to the ROI(s). In some embodiments, the parent molecules are bound with bodies that include caged affinity groups, which are protected by photo-liable protecting groups. The selected ROI(s) are exposed to photons so as to un-cage the affinity groups in the ROI(s), allowing them to bind to their respective affinity partner. In some embodiments, the process of amplification and/or binding to affinity partners within the ROI(s) is done on the fluidic device, in some embodiments, external to the device.

In another set of embodiments, the disclosed devices and methods allow for the segmentation of a long nucleic acid parent molecule into child molecules in such way that knowledge of the child's order relationship with the other children is maintained. In some embodiments, knowledge of both the order, and the relative distance in base-pairs of the children is maintained. In some embodiments, a physical map is interrogated and recorded for each child either before or after segmentation. By segmenting and cataloging the children, each of the children can then be individually processed, while maintain their physical contextual relationship from the originating parent, and to each other. For downstream applications, all, a random subset, or a selected subset of children may be processed, including amplification, sequencing, genotyping, or combination there-of. By maintaining parent knowledge, long range structural variations and phasing information can be elucidated in the context of maternal or paternal genomic lineage.

In another set of embodiments, the disclosed devices and methods allow of preparation of a long nucleic acid molecule such that regions are defined along the length of the molecule, and these regions all have unique barcodes, such that upon segmentation of the long molecule into children, the unique barcodes can help inform the origin of the child nucleic acid molecule within the originating parent. In some embodiments, the regional boundaries are selected at random, while in other embodiments, the regional boundaries are at least partially controlled. In the preferred embodiment the relationship between the barcode content and the region's physical boundaries within the originating parent molecule are known, however this not a requirement. In some embodiments the long nucleic acid parent molecule is first segmented into children that then comprise the regions, and then the barcodes are associated with the children, while maintaining knowledge of the children's relationship with other children. In some embodiments, the regions are defined along the length of the long nucleic acid parent molecule, and then the children segments are generated, whose boundaries can be random, or can be defined by the regional boundaries or some other criteria. In some embodiments, the barcodes are attached to universal primers, and the barcodes are then associated to a nucleic acid molecule via binding of the universal primer to the nucleic acid molecule. In some embodiments, the barcodes are associated with a nucleic acid molecule by physical confinement within a droplet. In some embodiments, the unique barcode constitutes a unique combination of barcodes.

In another set of embodiments, devices and methods are disclosed that enable the encapsulation of a long nucleic acid molecule in a single droplet in a manner that can be controlled, for example by the user or instrument controller, that does not rely on population statistics. In addition, further embodiments are disclosed that enable the individual tracking of single droplets whose contents are unique and known. Finally, embodiments are disclosed that enable blind and simultaneous injection of contents into droplets.

All fluidic device embodiments described in this disclosure fall into two major classes. The first class (“a confined fluidic device”) is comprised of at least a single fluidic elongation channel that is enclosed except for its fluidic connections, and is capable of presenting at least a portion of a long nucleic acid molecule in an elongated state for interrogation. In this class of devices, interrogation and exposure of the molecule to reagents and photons is performed while the molecule is surrounded by a solution, and unless specifically stated otherwise in the text, and can be manipulated and transported by a sufficiently large external force. This class of devices allows for dynamic control of the molecules within the device via interaction of the molecules with applied external forces and fluidic device elements within the fluidic device. The second class (“an open fluidic device”) includes a surface on which (or within a porous film on which) the molecule is at least partially placed or attached via molecular combing. In some embodiments the surfaces are patterned. In some embodiments there is a porous film on the surface. In this class of device, interrogation and initial exposure of the molecule to reagents is performed while the molecule is completely or partially immobilized on a surface, or at least partially contained within a porous film on the surface. This class of devices allows for direct interaction of the molecule with other devices external to the fluidic device, such as a fluid dispenser or contact probe.

Unless the class is stated explicitly, any reference to a “fluidic device” in this disclosure is referring to both classes of devices, regardless of grammar. Thus the statement: “a physical map is generated with the sample in the device” refers to both device classes, regardless of the use of the word “in”.

In one embodiment of the device, the input sample is a solution of suspended long nucleic acid molecules (macromolecules). In another embodiment of the device, the input sample is a solution of suspended packages, of which at least one package contains at least one long nucleic acid molecule, and the at least one long nucleic acid molecule is released from by package while in the device.

In one preferred embodiment of the device, the input sample solution and any associated reagent solutions required to operate the device, may be loaded via manual pipette dispensing or automated liquid handling systems. In one preferred embodiment of the microfluidic device, the operation of the device may be controlled by at least one control instrument, which in turn, may be controlled by a program or a person(s). Operation of the device by the control instrument can include manipulating the physical position and conformation of the package or long nucleic acid molecule via the application of external forces on said bodies, exposing the package or long nucleic acid molecule to various reagent compositions and concentrations for various time periods and temperatures, optically interrogating the package or long nucleic acid molecule, or their dynamic configuration changes to facilitate analysis of their composition or as part of a feedback system to control the operation of the device, or extracting desired packages or long nucleic acid molecules from the device. The microfluidic device and control instrument can interface in a number of ways. A non-exhaustive list includes: fluidic ports (both open and scaled), electrical terminals, optical windows, mechanical pads, heat pipes or sinks, inductance coils, fluid dispensing, surface scanning probes. A non-exhaustive list of potential functions the control instrument may perform on the device include: temperature monitoring, applying heat, removing heat, applying pressure or vacuum to ports, measuring vacuum, measuring pressure, applying a voltage, measuring a voltage, applying a current, measuring a current, applying electrical power, measuring electrical power, exposing the device to focused and/or unfocused electromagnetic waves, collecting the electromagnetic waves light generated or reflected from the device, in far or near-fiend setting, creating and measuring a temperature, electromagnetic force, surface energy or chemical concentration differential or gradient, dispensing liquid into a device well or port, or on the device surface, contacting the device surface or entity on the device surface with a contact probe.

In one embodiment, confirmation of the presence of the long nucleic acid molecule and control over its physical position within the device is modulated by the control instrument using a feedback controller system. Detection of the long nucleic acid molecule is via detection of a at least one optical, electromagnetic wave or electronic signal. In the preferred embodiment, the signal is an electromagnetic wave signal originating from a labelling body bound to said long nucleic acid molecule.

In one embodiment, the control instrument feedback control system at least in part utilizes as input information the identification of a physical map profile within the long nucleic acid molecule, or absence of a physical map profile within the molecule.

The control instrument may be centrally located, or have different parts distributed for different or redundant functions.

In order to run the operation software on the control instrument, and perform analysis for feedback control or interrogation data collection, a non-exhaustive list of potential options include: localized processing within the control instrument, adjacent processing via a direct communication connection, external processing via a network connection, or combination there-of. Various examples of processing modules include: a PC, a micro-controller, an application specific integrated micro-chip (ASIC), a field-programmable gate array (FPGA), a CPU, a GPU, System on Chip, a network server, cloud computing service, or combinations there-of.

The control instrument may include an imaging system, which may include any of the following types of imaging, or combinations there-of: fluorescent, cpi-florescent, total internal reflection fluorescence, dark field, bright field, nearfield/evanescent field, wave guide, zero mode waveguide, plasmonic signaling, super resolution, confocal, scattering, light sheet, structured illumination, stimulated emission depletion, stochastic activation super resolution, stochastic binding super resolution, multiphoton.

The control instrument may include at least one contract probe, preferably an atomic force microscope (AFM), that is capable of physically positioning the at least one control probe point at the desired x, y, z coordinates on the surface of the fluidic device.

The control instrument may include at least one fluidic dispensing tip that is capable of dispensing fluid drops at the desired x, y, z coordinates on the surface of the device, and in some embodiments, extracting fluid drops at the desired x, y, z coordinates on the surface of the fluidic device.

The control instrument may be able to fire multiple light sources simultaneously, or in series, and be able to image multiple colors simultaneously, or in series. If imaging multiple colors simultaneously, this may be done on different cameras, on a single camera but different regions of the sensor array, or on the same sensor of the same camera. In some embodiments, the wavelength of light fired by the control instrument is chosen so as to interact with the sample, the sample labeling body, or a functionalized surface in some way. Non limiting examples include: photo-cleaving of the nucleic acid, photo-cleaving photo-cleavable linkers, manipulating optical tweezers, activating photo-activated reactions, de-protecting photolabile protecting groups, IR thermal heating.

Instrumentation for photocleaving, when utilizing a photosensitization mechanism as described, delivers a dose of light of a wavelength adequate to excite the photosensitizing molecule, preferably 515 nm for TOTO-1 or most preferably 488 nm light in the case of YOYO-1. Light may be delivered via the excitation objective or via an external illumination device. When it is desired to selectively photoactivate a region of DNA, a focused light beam can be used, preferably a laser, most preferably a single-mode laser, where the focused spot is positioned at a known, fixed location relative to the field of view and the instrument possesses an XY stage capable of positioning the sample relative to the spot. More elaborate embodiments utilize a digital micromirror device and control system to project an arbitrary spot or plurality of spots at the sample. Further embodiments utilize scanning galvanometer mirrors to direct a spot to a particular region. The instrument can possess control elements, with or without active feedback, for delivering a known dose of light energy. Illumination by a focused 488 nm, 1.33 NA light cone will create an Airy disk with null diameter of 225 nm, corresponding to approximately ⅔ kb of fully stretched DNA.

The control instrument can possess additional refinements in order to minimize the spatial extent of the area subjected to photoactivation, and thereby minimize the genomic region subject to photoactivation. Such methods include stimulated emission depletion of the photosensitizer by performing simultaneous excitation with the existing wavelength of light while also irradiating the focal spot with a torus shaped focal spot of a wavelength of light that matches the emission wavelength of the photosensitizer, preferably 532 nm for TOTO-1 or most preferably 515 nm for YOYO-1. The torus shape is created by a diffractive optical element, spatial light modulator or equivalent method of inducing a spiral phase modulation to create an optical vortex. The photoactivation width can be decreased to 50-60 nm [Wollhofen, 2017], corresponding to approximately 175 bp of fully stretched DNA. Additional methods include the use of high index (n>1.55) hemispherical or aplanatic solid immersion lenses to create a tight focus of the incident light wave or waves, with or without stimulated emission depletion. An additional embodiment creates an in situ solid immersion lens in a silicon device by fabricating a spherical surface on the back side of the silicon device, positioned precisely opposite known fluidic features in the device. Silicon is highly absorptive in the visible wavelengths but this can be overcome with high incident light irradiance and, where applicable, cooling. Alternately a backside polished silicon substrate can be used in combination with a silicon hemisphere or sphere truncated to satisfy the aplanatic condition when added to the thickness of the silicon substrate.

The control instrument may have at least one photosensitive sensor, of which non-limiting examples include: CMOS camera, SCMOS camera, CCD camera, photomultiplier tube (PMT), Time Delay & Integration (TDI) sensor, photodiode, light dependent resistor, photoconductive cell, photo-junction device, photo-voltaic cell.

The control instrument may have at least on xy-stage, allowing for the imaging system to image different regions of the device, or other devices in the control instrument.

The control instrument may have 1 or more motors capable of adjusting the device's plane relative the control instrument's optical path, including z, tip, and tilt, based on an auto-focus feedback system, software analysis of image quality, device accessibility requirements, user access, or combination there-of.

The control instrument may capable of robotic transport of one or more fluidic devices to different parts of the control instrument.

In some embodiments the microfluidic device can include fiducial markers or alignment markers that can be used to enable visual alignment of the device either manually or with the control instrument's program. In some embodiments, there are multiple zones on the fluidic device, with each zone designed to physically isolate different input samples. In some embodiments, there are fiducial markers on the device that guide the user or automated dispensing system where on the device to dispense solution.

In one embodiment, the optical resolution of the physical map on the long nucleic acid molecule is improved by physically expanding and/or elongating the long nucleic acid molecule within at least one plane that is substantially normal to the optical axis used for interrogation. In some embodiments, this expansion is at least partially achieved via a timed exposure of the molecule to reagents (for example: enzyme that digest proteins and/or the nucleic acid) of controlled concentration, thus partially or fully releasing the nucleic acid strands from the chromatin structure. In some embodiments, this is at least partially achieved via the application of an applied force on the long nucleic acid molecule in the presence of physical obstacles, a porous medium, gel, or localized entropic traps within the reaction chamber that provide a retarding force, such that the largely counter-opposing retarding force and applied force on the long nucleic acid molecule act to elongate it. In some embodiments, this is at least partially achieved by introducing the long nucleic acid molecule to a fluidic environment within the device that increases the molecule's physical confinement within at least one dimension, causing the long nucleic acid molecule to physically expand within the non-confining dimension(s). In some embodiments, the molecule is transferred via an applied external force into a region of greater physical confinement. In other embodiments the fluidic environment in which the molecule occupies can be adjusted to become more confining to the molecule, for example with a channel wall that can be modulated by applying pressure or a vacuum to a neighboring channel that interfaces via a flexible wall [Unger, 1999, U.S. Pat. No. 7,144,616], or having a flexible channel walls that are comprised of, or are adjacent to, phase-change materials that can alter their shape to some stimulus [Hilber, 2016], or having channel walls attracted to each other, or repelled from each other with an electrostatic force via the application of an alternating electric field [Sounart, 2005], [Sounart, 2010]. In some embodiments, the long nucleic acid molecule experiences a compressive force with the application of a dielectrophoretic (DEP) force in a confined fluidic environment [Mashid, 2018, U.S. Pat. No. 10,307,769]. In some embodiments, a combination of any or all of these embodiment devices and methods are used to physically expand the long nucleic acid molecule, with any or all of these embodiment device methods under control of the control instrument, preferably using a feedback control system. In some embodiments, a physical mapping labelling method is used that allows for both the generation of karyotyping bands, and the generation of physical map along the length of a nucleic acid molecule. In this way, traditional karyotyping bands within the long nucleic acid molecule can be obtained, and then through manipulation of said long nucleic acid molecule via reagent exposures and/or physical confinement, portions of long nucleic acid molecules originating from the long nucleic acid molecule can be analyzed, identified, and compared to a reference. In some embodiments, the portions of long nucleic acid molecules remain connected to the originating long nucleic acid molecule during interrogation. In some embodiments, the portions are cleaved from the originating long nucleic acid molecule. In the preferred embodiment, the origination position within the long nucleic acid molecule from which the portion of long nucleic acid molecule originates from is monitored and recorded by the control instrument. In some embodiments, the originating position is selected, in preferred embodiments selected due to an analysis of a physical map on the originating long nucleic acid molecule.

The reagent materials and solutions that may be used include any that may be commonly used by someone trained in the art of performing cytogenetic analysis on chromosomes. Additional reagents may include various dyes or labeling bodies for physical mapping, FISH-probes, labelling bodies, methylation dyes, non-methylation dyes. In some embodiments, the flow of various reagents may always be in one direction. In other embodiments, the fluid flow may alternate. In some embodiments, there may be mixture of externally applied forces, for example a pressure driven reagent flow and an applied electrical field to manipulate the charged long nucleic acid molecule.

In some embodiments devices and methods, it is desired to interrogate the long nucleic acid molecule with labelling bodies bound to it that provide a signal similar or equivalent to a karyotyping banding profile. In some embodiments, the banding profile is generated by exposing the long nucleic acid molecules to various reagent compositions and concentrations, for various temperature and time periods. In some embodiments, reagent compositions can be chosen to produce banding patterns well recognized by those in the cytogenetics industry, including R band, Q bands, and G bands. To improve signal contrast, some embodiments will also include a counterstain. For a review of commonly used cytogenetic karyotyping dyes and bandings please refer to [Moore, 2001]. In addition to the traditional karyotyping dyes, in some embodiment devices and methods, it is desirable to generate banding patterns that are compatible with elongated single molecule mapping applications, such as the previously mentioned physical mapping methods. Furthermore, in some embodiments, the process of generating the bands can be controlled by the control instrument, using a feedback control system to monitor the process, and optimize the banding contrast for the desired application.

In some embodiments, the surface of at least one of the boundary walls of the fluidic device that constitutes the interrogation region are modified to change the surface energy or add functionalization to promote nucleic acid molecule immobilization with the said surface, or to provide reagents in support of a reaction. In some embodiments, the reagents are connected to the surface via a cleavable linker. In some embodiments, the functionalized regions are patterned. In some embodiments, a specific region of functionalization on the device surface is designed to immobilize a specific target of long nucleic acid molecule. In some embodiments, the specific target is a type of chromosome, or genomic region.

For all embodiments “prepare for interrogation” refers to the process of physically, chemically, or enzymatically manipulating the long nucleic acid molecule's conformation or structure and/or the bonding of labeling bodies to the molecule to enable interrogation of said molecule via a series of different reagent solution exposures of desired concentrations, times, and temperatures, via any of the device and method embodiments previously discussed. In the preferred embodiment, the labeling bodies on the long nucleic acid molecule comprise a physical map. In some embodiments, some of these preparations are performed beforehand, and thus “prepare for interrogation” in this context refers to the final steps necessary to enable interrogation of the molecules, as some steps have already been completed. For example, the input sample may consist of suspended droplets in solution, in which the contents of the droplet is a single cell that previously underwent processing, including: lysing, enzymatic digestion of proteins, and nucleic acid labeling of fluorescent labelling bodies to enable physical mapping. In some embodiments, at least some of the processes that define “prepare for interrogation” are done during interrogation, in some embodiments, as part of a feedback system. For example, it may be determined during interrogation that additional elongation is required, or a different physical conformation is desired, or the labeling bodies on the long nucleic acid molecule needs to be modified in some way (for example, add a new label of a different fluorescent color), or combinations there-of.

In some embodiments, the molecules can then be collected for further analysis, performed on the device, or external to the device via extraction of the molecules. Additional analysis can include, but not limited to: mapping, sequencing, array-CGH, SNP-arrays, 3D Mapping, amplification (PCR), or additional cytogenetic methods, such as hybridizing FISH probes.

All publications, patents, patent applications, and information available on the internet and mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

The terms used in this specification generally have their ordinary meanings in the art, within the context of the invention, and in the specific context where each term is used. Certain terms are discussed below, or elsewhere in the specification, to provide additional guidance to the practitioner in describing the devices and methods of the invention and how to make and use them. It will be appreciated that way. Consequently, alternative language and synonyms may the same thing can typically be described in more than one be used for any one or more of the terms discussed here. Synonyms for certain terms are provided. However, a recital of one or more synonyms does not exclude the use of other synonyms, nor is any special significance to be placed upon whether or not a term is elaborated or discussed herein. All publications, patent applications, patents, and other references mentioned herein are incorporated by reference. In the case of conflict, the present specification, including definitions, will control. In addition, the materials, methods, and examples are illustrative only and are not intended to be limiting.

The invention is also described by means of particular examples. However, the use of such examples anywhere in the specification, including examples of any terms discussed herein, is illustrative only and in no way limits the scope and meaning of the invention or of any exemplified term. Likewise, the invention is not limited to any particular embodiments described herein. Indeed, many modifications and variations of the invention will be apparent to those skilled in the art upon reading this specification and can be made without departing from its spirit and scope. The invention is therefore to be limited only by the terms of the appended claims along with the full scope of equivalents to which the claims are entitled.

As used herein, “about” or “approximately” in the context of a number shall refer to a range spanning +/−10% of the number, or in the context of a range shall refer to an extended range spanning from 10% below the lower limit of the listed range to 10% above the listed upper limit of the range.

The use of the term “or” in the claims is used to mean “and/or” unless explicitly indicated to refer to alternatives only or the alternatives are mutually exclusive, although the disclosure supports a definition that refers to only alternatives and “and/or.”

The words “a” and “an,” when used in conjunction with the word “comprising” in the claims or specification, denotes one or more, unless specifically noted.

Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural and singular number, respectively. Additionally, the words “herein,” “above,” and “below,” and words of similar import, when used in this application, shall refer to this application as a whole and not to any particular portions of the application.

The use of the term “combination” is used to mean a selection of items from a collection, such that the order of selection does not matter, and the selection of a null set (none), is also a valid selection when explicitly stated. For example, the unique combinations including the null of the set {A, B} that can be selected are: null, A, B, A and B.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF DRAWINGS

For all drawings, the use of roman numerals: i), ii), iii), iv) are to denote a passage of time.

FIG. 1 demonstrates 3 different non-limiting embodiments of generating a physical map along the length of a long nucleic acid molecule. (A) is a physical map generated by cleaving the molecule at known recognition sites producing an ordered pattern of lengths. (B) is a physical map generated by attaching label bodies at known recognition sites producing an ordered pattern of segments. (C) is a physical map generated by attaching label bodies along the length of molecule in a manner such the density of the labeling bodies correlates with the underlying AT/CG ratio.

FIG. 2 demonstrates an enclosed fluidic device and method for generating combed linearly elongated nucleic acid molecule in parallel fashion, with (i) showing the molecules being flown into an enclosed channel, and with (ii) showing said molecules after the roof is removed from the channel.

FIG. 3 demonstrates different, non-limiting embodiments of confined and non-confined channel types within a fluidic device.

FIG. 4 demonstrates various fluid device embodiments of a deformable object encountering entropic barriers, slopes and traps. (A) an object encounters an entropic barrier. (B) an object escapes from an entropic trap. (C) an object encounters an entropic slope. (D) an object encounters an entropic trap.

FIG. 5 demonstrates a deformable object encountering an entropic barrier.

FIG. 6 demonstrates a method of identifying, and then separating two ROIs from their shared parent molecule.

FIG. 7 demonstrates various device and method embodiments for directing a flow of reagents to at least one specific ROI on a molecule: (A) An ROI of an elongated molecule in an elongation channel is exposed to a cross-flow of reagents, (B) An ROI of molecule exposed to a cross-flow of reagents, while the non-ROI portion of the molecule resides in an entropic trap, or behind an entropic barrier. (C) An ROI of a molecule exposed to a cross-flow of reagents, while the non-ROI portion is contained within entropic traps that are shielded from the laminar cross flow of reagents. (D) An ROI of a molecule exposed to a cross-flow of reagents, where-by the regent cross flow is sandwiched between two other cross flows such that the effective width of the reagent cross flow can be controlled.

FIG. 8 demonstrates various device and method embodiments for a tail portion of a long nucleic acid molecule to reagents: (A) with the non-exposed portion of the long nucleic acid molecule retained by a retarding force. (B) with the non-exposed portion of the long nucleic acid molecule retained by an entropic barrier. (C) with the non-exposed portion of the long nucleic acid molecule retained by physical obstacles.

FIG. 9 demonstrates a method of generating a captured primer

FIG. 10 demonstrates a method of selectively activating primers along the length of a long nucleic acid molecule, where-by (A) demonstrates an example non-active universal primer with a barcode, and (B) demonstrates a method for activating the primers within a ROI for selective amplification.

FIG. 11 demonstrates a method of selectively un-caging affinity groups contained within bound bodies on a long nucleic acid molecule, where-by said bound bodies include a photo-liable protecting group.

FIG. 12 demonstrates a method and device of selectively exposing an ROI along a long nucleic acid molecule in a confined fluidic device, such that un-caged affinity groups in the ROI become uncaged and can then bind with their respective affinity partners.

FIG. 13 demonstrates a device and method embodiment for exposing an ROI region of a molecule elongated in a gel to a reaction. (A) a long nucleic molecule is elongated in elongation channel of confined fluidic device and gelled with reagents. (B) an embodiment method where-by post-gelling, the ROI region is exposed to IR to melt the gel. (C) an embodiment method where-by post-gelling, the ROI region is exposed to a wavelength of light to photo-activate the reagents.

FIG. 14 demonstrates a device and method for selectively exposing an ROI within a long nucleic acid molecule on an open fluidic device to a solution containing reagent using a dispenser.

FIG. 15 demonstrates a device and method for selectively exposing multiple ROIs within a long nucleic acid molecule on an open fluidic device to different solution compositions using a dispenser.

FIG. 16 demonstrates a device and method for selectively exposing an ROI within a long nucleic acid molecule on an open fluidic device to a solution using a dispenser, where-by the fluidic device includes patterned wells that allow for the solution drop containment around the ROL.

FIG. 17 demonstrates (A) a device and method embodiment for selectively exposing an ROI region of a combed molecule in a gel on a surface of an open fluidic device to IR, and (B) a device and method embodiment for selectively exposing an ROI region of a combed molecule on a surface of an open fluidic device to photons.

FIG. 18 demonstrates various device and method embodiments allow for the targeted enzymatic cleaving of long nucleic acid molecules in at least a partially elongated state within a confined fluidic device, including (A) the targeted flow of cleaving reagents to a specific region of a molecule contained within an elongation channel, (B) and (C) the targeted flow of cleaving reagents to a specific region of a molecule excluded from an entropic trap.

FIG. 19 shows various device and method embodiments allowing for the targeted photo-cleaving of long nucleic acid molecules in at least a partially elongated state within a confined fluidic device, including (A) a molecule elongated in an elongation channel, (B) a molecule elongated by an applied external force with physical obstacles interacting with the molecule, (C) a molecule elongated in an elongation channel with an applied external force, while a retarding force is applied to the molecule, (D) a molecule contained within two entropic traps, with the connecting portion of the molecule between the traps located in an elongation channel.

FIG. 20 demonstrates a device and method embodiment for capturing an ROI within an entropic trap, and then disposing the non-ROI parent molecule material.

FIG. 21 demonstrates a device and method embodiment for capturing an ROI within at least one entropic trap of an entropic trap array, and then disposing the non-ROI parent molecule material.

FIG. 22 demonstrates a device and method embodiment for capturing long nucleic acid molecule in a gel in an elongated state, identifying an ROI, and then photo-cleaving and removing said ROI to separate it from the parent.

FIG. 23 demonstrates a method and device of selectively exposing an ROI along a long nucleic acid molecule in a confined fluidic device, such that un-caged affinity groups in the ROI become uncaged and can then bind with their respective affinity partners, and in addition, separating the ROI from the parent molecule by photo-cleaving.

FIG. 24 demonstrates a device and method embodiment for capturing an ROI from a combed parent molecule by photo-cleaving the ROL, and then capturing the ROI using a contact probe.

FIG. 25 demonstrates a device and method embodiment for capturing an ROI from a combed parent molecule by photo-cleaving the boundaries of the ROI, and then re-suspending the ROI in a dispensed liquid drop, and then extracting the drop from the surface.

FIG. 26 demonstrates a device and method embodiment for capturing an ROI from a parent molecule combed on a surface of patterned wells by photo-cleaving the boundaries of the ROI, and dispensing a solution so that the ROI is re-suspending in solution, and the solution drop is contained in a well.

FIG. 27 demonstrates a device and method embodiment for capturing an ROI from a parent molecule by un-caging affinity groups bound to the ROL, photo-cleaving the boundaries of the ROI.

FIG. 28 demonstrates a method embodiment assigning known barcodes to child molecules whose origin within the parent molecule is known.

FIG. 29 demonstrates a device and method embodiment where-by a parent molecule is segmented into children molecules by means of an entropic trap array and photo-cleaving.

FIG. 30 demonstrates a device and method embodiment where-by a parent molecule is segmented into children molecules by means of an entropic trap array and photo-cleaving, where-by the physical map of each child is generated and recorded.

FIG. 31 demonstrates a device and method embodiment where-by a long nucleic acid parent molecule is segmented into children molecule, each contained in a water-in-oil droplet, by first segmenting the children by entropic traps and photo-cleaving, and then displacing the aqueous solution with an oil based solution. (B) is a cross-section of (A).

FIG. 32 demonstrates a device and method embodiment where-by a droplet (here containing a long nucleic acid molecule) can be released from an entropic trap by removing the entropic trap barrier (here by adjusting the channel confining dimensions.

FIG. 33 demonstrates a method embodiment where-by barcodes attached to primers are bound to a long nucleic acid molecule, with a unique, and known barcode for each region of the molecule.

FIG. 34 demonstrates a method embodiment where-by a long nucleic acid molecule is bound to universal primers with unique barcodes in each region, and then said molecule is fragmented.

FIG. 35 demonstrates a device and method embodiment where-by barcodes attached to primers are bound to a long nucleic acid molecule by bringing the molecule into proximity of an array of barcode pads within a fluidic device.

FIG. 36 demonstrates a device and method embodiment where-by barcodes attached to primers are bound to a long nucleic acid molecule by combing the molecule over an array of barcode pads on a surface.

FIG. 37 demonstrates a device and method of forming a droplet that contains a long nucleic acid molecule.

FIG. 38 demonstrates a device and method of injecting a long nucleic molecule into a droplet.

FIG. 39 demonstrates a device and method of displacing water with oil in the droplet channel such that a long nucleic acid molecule can be brought to the injection point, and then injected into a droplet.

FIG. 40 demonstrates a device and method of maintaining a droplet at an injector with either (A) an entropic barrier for the droplet, or (B) an entropic trap for the droplet.

FIG. 41 demonstrates a device and method of trapping multiple droplets at multiple injection points.

FIG. 42 demonstrates a method of using a long nucleic acid molecule's physical map as a unique signature.

### DETAILED DESCRIPTIONS

The following definitions will be used in this disclosure:

### Definitions

Sample. The term “sample,” as used herein, generally refers to a biological sample of a subject which at least partially contains nucleic acid originating from said subject. The biological sample may comprise any number of macromolecules, for example, cellular long nucleic acid molecules. The sample may be a cell sample. The sample may be a cell line or cell culture sample. The sample can include one or more cells. The sample can include one or more microbes. The biological sample may be a nucleic acid sample. The biological sample may be derived from another sample. The sample may be a tissue sample, such as a biopsy, core biopsy, needle aspirate, or fine needle aspirate. The sample may be a fluid sample, such as a blood sample, urine sample, or saliva sample. The sample may be a skin sample. The sample may be a cheek swab. The sample may be a plasma or serum sample. The sample may be a cell-free or cell free sample. A cell-free sample may include extracellular polynucleotides. Extracellular polynucleotides may be isolated from a bodily sample that may be selected from the group consisting of blood, plasma, serum, urine, saliva, mucosal excretions, sputum, stool and tears.

Nucleic Acid. The terms “nucleic acid”, “nucleic acid molecule”, “oligonucleotide” and “polynucleotide”, “nucleic acid polymer”, “nucleic acid fragment”, “polymer” are used interchangeably and refer to a polymeric form of nucleotides of any length, either deoxyribonucleotides or ribonucleotides, or analogs thereof. The terms encompass, e.g., DNA, RNA and modified forms thereof. Polynucleotides may have any three-dimensional structure, and may perform any function, known or unknown. Non-limiting examples of polynucleotides include a gene, a gene fragment, exons, introns, messenger RNAs (mRNA), transfer RNAs, ribosomal RNAs, lncRNAs (Long noncoding RNAs), lincRNAs (long intergenic noncoding RNAs), ribozymes, cDNA, ecDNAs (extrachromosomal DNAs) , artificial minichromosomes, cfDNAs (circulating free DNAs), ctDNAs (circulating tumor DNAs), cffDNAs (cell free fetal DNAs), recombinant polynucleotides, branched polynucleotides, plasmids, vectors, isolated DNA of any sequence, control regions, isolated RNA of any sequence, nucleic acid probes, and primers.

Unless specifically stated otherwise, the nucleic acid molecule can be single stranded, double stranded, or a mixture there-of. For example, there may be hairpin turns or loops.

Long Nucleic Acid Molecule. Unless specifically stated otherwise, a “long nucleic acid fragment” or “long nucleic acid molecule” is double strand nucleic acid of at least 5 kbp in length, and is thus a kind of macromolecule, and can span to an entire chromosome. It can originate from any source, man-made or natural, including single cell, a population of cells, droplets, an amplification process, etc. It can include nucleic acids that have additional structure such as structural proteins histones, and thus includes chromatin. It can include nucleic acid that has additional bodies bound to it, for example labeling bodies, DNA binding proteins, RNA.

Child Molecule. Unless specifically stated otherwise, a “child molecule” or “child fragment” is a long nucleic acid molecule that has been separated from a larger originating “parent” long nucleic acid molecule.

Hybridization. As used herein, the terms “hybridization”, “hybridizing,” “hybridize,” “annealing,” and “anneal” are used interchangeably in reference to the pairing of complementary or substantially complementary nucleic acids. Hybridization and the strength of hybridization (i.e., the strength of the association between the nucleic acids) is influenced by such factors as the degree of complementary between the nucleic acids, stringency of the conditions involved, the Tm (melting temperature) of the formed hybrid, and environmental conditions such as temperature and pH. “Hybridization” methods involve the annealing of one nucleic acid to another, complementary nucleic acid, i.e., a nucleic acid having a complementary nucleotide sequence.

Pairing can be achieved by any process in which a nucleic acid sequence joins with a substantially or fully complementary sequence through base pairing to form a hybridization complex. For purposes of hybridization, two nucleic acid sequences are “substantially complementary” if at least 60% (e.g., at least 70%, at least 80%, or at least 90%) of their individual bases are complementary to one another.

In the context of this document, where hybridization occurs between nucleic acid strand and a double-stranded nucleic acid molecule, it should be understood that such hybridization is being done under conditions of either partial or full denaturation of the double-stranded nucleic acid molecule, unless otherwise specifically stated.

Labeling body. A “labelling body” used herein is a physical body that can bind to a nucleic acid molecule, which can be used to generate a signal, for example with a fluorescent imaging device and/or a constriction device, that differs from a signal (or lack there-of) that would be generated by said nucleic acid without said body. A labelling body may be a fluorescent intercalating dye that when bound to nucleic acid, can be used in a fluorescent imaging system to identify the presence of said nucleic acid. In another example, a labelling body may by a compound that binds specifically to methlyated nucleotides, and gives a current blockade signal when transported through a nanopore, thus reporting a signal as to said molecule's methlyation state. In another example, a fluorescent probe specifically hybridized to a sequence of a nucleic acid, thus providing confirmation with a fluorescent imaging system that the sequence is present on said nucleic acid. In some cases, the absence of the labelling body, is itself the signal. In some cases, the labelling body is not physically attached to the nucleic molecule at the time of assessing said nucleic molecule and labelling body. For example, a labelling body may be attached to a nucleic acid molecule via a cleavable linker. At the desired time, the linker is cleaved, releasing said labelling molecule which is then detected.

Interrogation. “Interrogation” is a process of assessing the state of a labeling body on a nucleic acid by measuring a signal generated directly, or indirectly from the labeling body. It may be a binary assessment, such as the labeling body is present, or not. It may be quantitative such as how many labeling bodies are present on a molecule. It may be a trace of the density and/or physical count of labeling bodies along the length the molecule in relation to the molecule's physical structure. The signal may be fluorescent, electrical, magnetic, physical, chemical. The signal may be analog or digital in nature. For example, the signal may be an analog density profile of the labeling body along the length of the nucleic acid. Non exhaustive examples of different interrogation methods include fluorescent imaging, bright-field imaging, dark-field imaging, current, voltage, power, capacitive, inductive, or reactive measurement, nanopore sensing (both column blockade through the pore, and tunneling across the pore), chemical sensing (eg: via a reaction), physical sensing (eg: interaction with a sensing probe), SEM, TEM, STM, SPM, AFM. In addition, combinations of different labeling bodies and interrogation methods are also possible. For example: fluorescent imaging of an intercalating dye on a nucleic acid, while translocating said nucleic acid through a nanopore and measuring the pore current.

Sequence. The term “sequence” or “nucleic acid sequence” or “oligonucleotide sequence” refers to a contiguous string of nucleotide bases and in particular contexts also refers to the particular placement of nucleotide bases in relation to each other as they appear in an oligonucleotide. Sequencing can be performed by various systems currently available, such as, with limitation, a sequencing system by Illumina, Pacific Biosciences, Oxford Nanopore, Life Technologies (Ion Torrent), BGI.

Structural Variations. As used herein, “structural variation” or “SV” is the variation in structure of an organism's chromosome with respect to a genomic reference. These variations include a wide variety of different variant events, including insertions, deletions, duplications, retrotransposons, translocations, inversions short and long tandem repeats, and the like. These structural variations are of significant scientific interest, as they are believed to be associated with a range of diverse genetic diseases. In general, the operational range of structural variants includes events >50 bp, while the “large structural variations” typically denotes events >1,000 bp or more. The definition of structural variation does not imply anything about frequency or phenotypical effects.

Genomic Reference. A “genomic reference” or “reference” is any genomic data set that can be compared to another genomic data set. Any data formats may be employed, including but not limited to sequence data, karyotyping data, methylation data, genomic functional element data such as cis-regulatory element (CRE) map, primary level structural variant map data, higher order nucleic acid structure data, physical mapping data, genetic mapping data, optical mapping data, raw data, processed data, simulated data, signal profiles including those generated electronically or fluorescently. A genomic reference may include multiple data formats. A genomic reference may represent a consensus from multiple data sets, which may or may not originate from different data formats. The genomic reference may comprise a totality of genomic information of an organism or model, or a subset, or a representation. The genomic reference may be an incomplete representation of the genomic information it is representing.

The genomic reference may be derived from a genome that is indicative of an absence of a disease or disorder state (e.g., germline nucleic acid) or may be derived from a genome that is indicative of a disease or disorder state (e.g., cancer nucleic acid, nucleic acid indicative of an aneuploidy, etc.). Moreover, the genomic reference (e.g., having lengths of longer than 100 bp, longer than 1 kb, longer than 100 kb, longer than 10 Mb, longer than 1000 Mb) may be characterized in one or more respects, with non-limiting examples that include determining the presence (or absence) of a particular feature, determining the presence (or absence) of a particular haplotype, determining the presence (or absence) of one or more genetic variations (e.g., structural variations (e.g., a copy number variation, an insertion, a deletion, a translocation, an inversion, a retrotransposon, a rearrangement, a repeat expansion, a duplication, etc.), single nucleotide polymorphisms (SNPs), etc.) and combinations thereof. It should be noted that any change from the genomic reference could be of interest. As such, for all examples “presence” and “absence” refers not only to being present or absent from the genomic reference in its entirety, but also present or absent from a particular region of genomic reference, as defined by the neighboring genomic content. Moreover, any suitable type and number of sequence characteristics of the genomic reference can be used to characterize the sequence of the sample nucleic acid. For example, one or more genetic variations (or lack thereof) or structural variations (or lack thereof) of a reference nucleic acid sequence may be used as a sequence signature to identify the reference nucleic acid as indicative of the presence (or absence) of a disorder or disease state. Based on the characterization of the reference nucleic acid sequence utilized, the sample nucleic acid sequence can be characterized in a similar manner and further characterized/identified as derived (or not derived) from a nucleic acid indicative of the disorder or disease based upon whether or not it displays a similar character to the reference nucleic acid sequence.

In some cases, the genomic reference is a physical map. This can be generated in any number of ways, including but not limited to: raw single molecule data, processed single molecule data, an in-silico representation of a physical map generated from a sequence or simulation, an in-silico representation of a physical map generated by assembling and/or averaging multiple single molecule physical maps, or combination there-of. For example, based on a known, or partially known sequence, a simulated in-silico physical map can be generated based on the method of generating a physical map used. In an embodiment where-by the physical map comprises labelling bodies at known sequences, a discrete ordered set of segment lengths in base-pairs can be generated. In an embodiment where-by the physical map comprises a melt-map, a continuous analog signal of labeling signal density along the sequence length in base-pairs, based on simulated local melting temperatures for a desired partial de-naturing condition can be generated [Tegenfeldt, 2008, U.S. Pat. No. 9,597,687].

In some cases, the genomic reference is data obtained from microarrays (for example: DNA microarrays, MMChips, Protein microarrays, Peptide microarrays, Tissue microarrays, etc), or karyotypes, or FISH analysis. In some cases, the genomic reference is data obtained from 3D Mapping technologies.

In some cases, characterizations of the comparison with the genomic reference may be completed with the aid of a programmed computer processor. In some cases, such a programmed computer processor can be included in a computer control system.

Physical Mapping. “Physical mapping” or “mapping” of nucleic acid comprises a variety of methods of extracting genomic, epigenomic, functional, or structural information from a physical fragment of long nucleic acid molecule. As a general rule, the information obtained is of a lower resolution than the actual underlying sequence information, but the two types of information are correlated (or anti-correlated) spatially within the molecule, and as such, the former often provides a ‘map’ for sequence content with respect to physical location along the nucleic acid. In some embodiments, the relationship between the map and the underlying sequence is direct, for example the map represents a density of AG content along the length of the molecule, or a frequency of a specific recognition sequence. In some embodiments, the relationship between the map the underlying sequence is indirect, for example the map represents the density of nucleic acid packed into structures with proteins, which in turn is at least partially a function of the underlying sequence. In the preferred embodiment, the physical map is generated by interrogating labeling bodies that are bound along an elongated portion of a long nucleic acid molecule's major axis. There are a multitude of physical map methods.

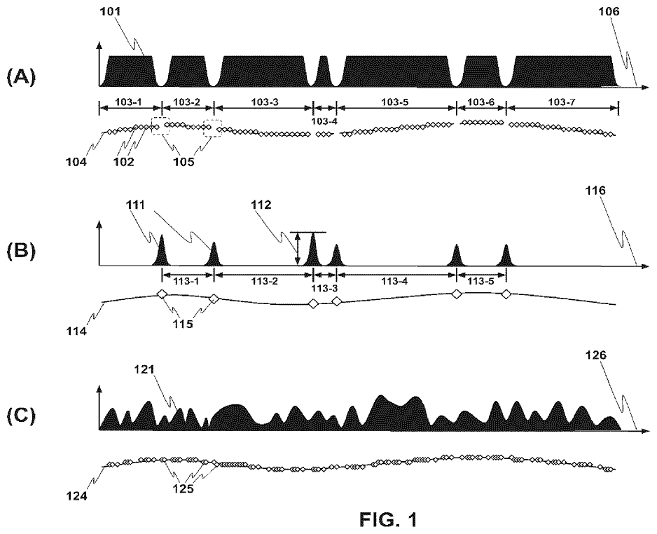

The first and most widely used form of physical mapping is karyotyping, where-by metaphase chromosomes are treated with a stain process that preferentially binds to AT or CG regions, thus producing ‘bands’ that correlate with the underlying sequence of the nucleic acid [Moore, 2001]. However, the resolution of such a process is quite poor, about 5-10 Mbp, due to the condensed nature of nucleic acid being imaged, so more recent methods of physical karyotyping have improved upon the resolution of physical mapping using elongated nucleic acid free of any bound structural supporting proteins, often during the so-called interphase of genomic DNA. With the nucleic acid in an elongated state, physical maps have been generated by imaging nucleic acid digested at known restriction sites [Schwartz, 1988, U.S. Pat. No. 6,147,198] (eg: see FIG. 1(A)), imaging attached fluorescent probes at nicking sites [Xiao, 2007] (eg: see FIG. 01(B)), imaging the fluorescent signature of a nucleic acid molecule's methlyation pattern [Sharim, 2019], imaging the fluorescent signature of a chromatin's histone [Riehn, 2011], electrical detection of bound probes to a nucleic acid through a sensor [Rose, 2013, 2014/0272954], and electrical detection of the methylation signature on a nucleic acid using a nanopore sensor [Rand, 2017]. Such non-condensed interphase nucleic acid polymer strands are often extracted from bulk solution of pooled samples with many potentially heterogeneous cells.

Another method of physical mapping is to measure the AT/CG relative density or local melting temperature along the length of an elongated nucleic molecule (eg: see FIG. 1(C)). Such a signal can either be used to compare against other similar maps, or against a map generated in-silico from sequence data. There are many ways of generating such a signal. For example, the signal can be fluorescent or electrical in nature. Nucleic acid can be uniformly stained with an intercalating dye, and then partially melted resulting in the relative loss of dye in regions of rich AT content [Tegenfeldt, 2009, U.S. Pat. No. 10,434,512]. Another method is to expose double stranded nucleic acid to two different species that compete to bind to the nucleic acid. One species is non-fluorescent and preferentially binds to AT rich regions, while the other species is fluorescent and has no such bias [Nilsson, 2014]. Yet another method is to use two different color dyes that differentially label the AT and CG regions.

FIG. 1 demonstrates a variety of different embodiments for generating and interrogating a LNAM's physical map. In FIG. 1(A), a physical map of a long nucleic acid molecule 104 is generated by cleaving the molecule at particular sequence sites (eg: recognition sites for restriction enzymes) thus resulting in gaps 105 where the cleaving event took place. Along the length of a molecule, a dye is attached non-specifically (eg: using an intercalating dye) such that child molecules from the originating the parent molecule can be interrogated to generate a signal 101 that follows the physical length (0106) of the parent molecule. The signal can then be used determined the lengths and order of the individual child molecules {103-x}, and thus generating the parent molecule's physical map. In most embodiments of this method, the parent molecule is combed onto a surface and then cleaved, so at to maintain physical proximity and relative order of the child molecules. However, such an embodiment could also be implemented in at least a partially elongated state within an elongating channel of a confined fluidic device such that the order of the child molecules can be interrogated [Ramsey, 2015, U.S. Pat. No. 10,106,848]. In some embodiments, a mixture of different cleaving sites may be used simultaneously.

In FIG. 1(B), a physical map of a long nucleic acid molecule 114 is generated by sparsely binding label bodies 115 along the length of the molecule that bind to the nucleic acid in a way such that the binding sites are correlated (or anti-correlated) with a specific target, or set of specific targets. In some methods, the labeling body is bound directly to a sequence target, for example, with a sequence-specific binding motif. In some methods, the labeling body is bound indirectly, for example: a sequence specific nick is generated, followed by incorporation of nucleotides starting at the nick site, some of which may be capable of generating a signal. The long nucleic acid molecule with labeling bodies is interrogated, generating signals 111 from the label bodies 115 along the physical length of the molecule 116. The distance between the signals, a collection of lengths and orders {113-x} then represents the molecule's physical map. In some embodiments, further information can be generated by also interpreting the relative magnitudes of the signals 112 from the various labeling sites. When fluorescent interrogation is used, different color labeling bodies can be used to represent different specific sites. In some embodiments, such as with FISH, the presence of a single signal is the ‘physical map’, as it suggests the presence of absence of the specific target.

In FIG. 1(C), a physical map of a long nucleic acid molecule 124 is generated by densely binding labeling bodies 125 along the length of the molecule, such that the binding pattern correlates (or anti-correlates) with the underlying physical sequence content of the molecule. For example, the relative AT/CG content, or the relative melting temperature, or the relative density of methylated CGs. Due to the dense nature of the labeling bodies in this method, the physical map is not a collection of lengths and orders, but rather an analog signal 121 that varies in intensity along the physical length of the molecule 126.

The method of interrogation to generate a physical map is typically fluorescent imaging, however different embodiments are also possible, including a scanning probe along the length of a combed molecule on a surface, or a constriction device that measures the coulomb blockade current through or tunneling current across the constriction as the molecule translocate through.

Unless specifically stated otherwise, a physical map refers to any of the previously mentioned methods, including combinations there-of. For example, a long nucleic acid molecule may have a physical map generated from the AT/TC density with a fluorescent labelling body along the length of the molecule, and then also have a physical map generated from the methylation profile along the length of the molecule by constriction device as the molecule is transported through said constriction device.

### Elongated Nucleic Acid

The majority of physical mapping methods that use fluorescent imaging or electronic signals to extract a signal related to the underlying genomic, structural, or epigenomic content employ some form of method to at least locally ‘elongate’ the long nucleic acid molecule such that the resolution of the physical mapping in the region of elongation can be improved, and disambiguates reduced. A long nucleic acid molecule in its natural state in a solution will form a random coil. Thus, a variety of methods have been developed to ‘uncoil’ and elongate the molecule to allow for a variety of applications, particularly for the interrogation of the molecule to generate a physical map.

Long nucleic acid molecules can be elongated on a solid surface by flowing a solution of nucleic acid on a substrate prepared such that the nucleic acid can bind to it. By binding a portion of the nucleic acid, and allowing the solution to flow, the nucleic acid is pulled taut by the opposing forces, and ultimately comes into full contact with the surface [Bensimon, 1997, U.S. Pat. No. 7,368,234], a technique typically called ‘combing’ DNA. Alternatively, the nucleic acid can remain un-bound to the surface except for the end of the molecule, again allowing a fluid flow to pull the nucleic acid taut [Gibb, 2012]. Alternatively, nucleic acid can be elongated by the sheer force of dynamic focusing laminar flows of aqueous solution [Chan, 1999, U.S. Pat. No. 6,696,022], or confining nanochannels where-by the lowest energy state within is one of an elongated state [Tegenfeldt, 2005]. In addition, a long nucleic acid molecule can be elongated by applying two opposing forces on the molecule that pull the molecule taut, typically in a microfluidic device. Examples include applying an external force on a long nucleic acid molecule in a presence of physical features to which the nucleic acid interacts with, thus generating a retarding force on the molecule that opposes the applied external force [Volkmuth, 1992]; or positioning the molecule in a fluidic device in which it is simultaneously exposed to two opposing externally applied forces thus generating a hydrodynamic trap [Tanyeri, 2011].

Once at least a portion of the long nucleic acid molecule has been elongated, depending on the circumstance of the method and device used to elongate it, the nucleic acid may be able to return to its natural random coiled state when an external force is removed. For example, cessation of a fluid flow used to elongate a nucleic acid molecule will result in the molecule reverting to a random coil. However, if the nucleic acid is held within a physically confining environment, then the nucleic acid may be able to retain at least a portion of the elongated state when an external force is removed [Dai, 2016].

Unless specifically stated otherwise, an ‘elongated’ or ‘partially elongated’ nucleic acid is a long nucleic acid fragment for which at least one segment of the major axis of the molecule comprising at least 1 kb can be projected against a 2D plane, and does not overlap with itself. For clarity, for embodiments where-by long nucleic acid includes additional structure, for example as when the nucleic acid is contained in chromatin, compacted with histones, the major axis refers to the larger chromatin molecule, not the nucleic acid strand itself. Therefore statements in this disclosure such as “along the length of the molecule” when referring to long nucleic acid molecules, refers to along the length of the major axis.

3D Mapping. In this document, “3D mapping” refers to protocols that involve capturing the proximity relationship of at least two strands of nucleic acid, either of the same chromosome or not. For reference [Kempfer, 2020] reviews these various techniques, of which a non-exhaustive list includes the following: 3C, 4C, 5C, Hi-C, TCC, PLAC-seq, ChIA-PET, Capture-C, C-HiC, Single-Cell HiC, GAM, SPRITE, ChIA-Drop.

Barcode. As used herein a “barcode” is a short nucleotide sequence (e.g., at least about 4, 6, 8, 10, 12, 14, 16, 18, 20, 25, 30, 35 nucleotides long) that encodes information. The barcodes can be one contiguous sequence or two or more noncontiguous sub-sequences. Barcodes can be used, e.g., to identify molecules in a partition or a bead to which an oligonucleotide is attached. In some embodiments, a bead-specific barcode is unique for that bead as compared to barcodes in oligonucleotides linked to other beads. In another example, a nucleic acid from each cell can be distinguished from nucleic acid of other cells due to the unique “cellular barcode.” Such partition-specific, cellular, or bead barcodes can be generated using a variety of methods. In some cases, the partition-specific, cellular, or particle barcode is generated using a split and mix (also referred to as split and pool) synthetic scheme, for example as described in [Agresti, 2014, 2016/0060621]. More than one type of barcodes can in some embodiments be in the oligonucleotides described herein.

In some embodiments, the information associated with the barcode may be an identification of a single, a particular, a type, a sub-set, a specific selection, a random selection, a group of body, where the body may be a molecule, a higher-order nucleic acid structure, an organelle, a sample, a subject. In some embodiments, the information associated with the barcode may be a process, a time-stamp, a location, a relationship with another body and/or barcode, an experiment id, a sample id, or an environmental condition. In some embodiments multiple information content may be stored in the barcode, using any encoding technique.

In some embodiments the barcode is single strand. In some embodiments the barcode is double-stranded. In some embodiments, the barcode has both single and double strand components. In some embodiments the barcode is at least partially comprised of 2D and/or 3D structures, for example hairpins or a DNA origami structure.

In some embodiments, the information encoded in the barcode is done using error checking and/or error-correcting techniques to ensure the validity of the information stored within. For example, the use of hamming codes. In some cases where multiple information content is stored in the barcode, the separate pieces of information are encoded separately with their respective nucleotides within the barcodes. In other cases, the nucleotides can be shared using an encoding scheme. In some cases, compression techniques can be used to reduce the number of nucleotides needed.

In some embodiments, the information encoded in the barcode includes uniquely identifying the molecule to which it is conjugated. These types of barcodes are sometimes referred to as “unique molecular identifiers” or “UMIs”. In still other examples, primers can be utilized that contain “partition-specific barcodes” unique to each partition, and “molecular barcodes” unique to each molecule. After barcoding, partitions can then be combined, and optionally amplified, while maintaining “virtual” partitioning based on the particular barcode. Thus, e.g., the presence or absence of a target nucleic acid comprising each barcode can be counted or tracked (e.g. by sequencing) without the necessity of maintaining physical partitions.

The length of the barcode sequence determines how many unique barcodes can be differentiated. For example, a 1 nucleotide barcode can differentiate 4, or fewer, different samples or molecules; a 4 nucleotide barcode can differentiate 256 samples or less; a 6 nucleotide barcode can differentiate 4096 different samples or less; and an 8 nucleotide barcode can index 65,536 different samples or less.

In some embodiments, the barcode sequences are designed or randomly generated using a selection software for choosing barcodes that are: without hairpin, or containing even base composition (15%-30% A, T, G and C), or without homopolymers (default allows 3 bases of same nucleotides), or without simple repeats, or without low complexity sequences, or not identical to common vector or adaptor sequences. Furthermore, barcodes can be designed to be unique even if there are 3 mismatch sequencing errors.

Barcodes are typically synthesized and/or polymerized (e.g., amplified) using processes that are inherently inexact. Thus, barcodes that are meant to be uniform (e.g., a cellular, particle, or partition-specific barcode shared amongst all barcoded nucleic acid of a single partition, cell, or bead) can contain various N−1 deletions or other mutations from the canonical barcode sequence. Thus, barcodes that are referred to as “identical” or “substantially identical” copies can in some embodiments include barcodes that differ due to one or more errors in, e.g., synthesis, polymerization, or purification errors, and thus can contain various N−1 deletions or other mutations from the canonical barcode sequence. However, such minor variations from theoretically ideal barcodes do not interfere with the methods, compositions, and kits described herein. Therefore, as used herein, the term “unique” in the context of a particle, cellular, partition-specific, or molecular barcode encompasses various inadvertent N−1 deletions and mutations from the ideal barcode sequence. In some cases, issues due to the inexact nature of barcode synthesis, polymerization, and/or amplification, are overcome by oversampling of possible barcode sequences as compared to the number of barcode sequences to be distinguished (e.g., at least about 2-, 5-, 10-fold or more possible barcode sequences), or by using error correction encoding techniques. The use of barcode technology is well known in the art, see for example [Shiroguchi, 2012] and [Smith, 2010]. Further methods and compositions for using barcode technology include those described in [Agresti, 2014, 2016/0060621].

In some embodiments, at least a portion of the barcode can also be used as a primer binding site. In some embodiments, the primer binding site is for a PCR primer. In some embodiments, all barcodes that form a set of unique barcodes contain within said barcodes a globally identical primer binding site, such that a single primer sequence can be used to bind to all barcodes. In some embodiments, the primer will be the complement sequence of the primer binding site. In other embodiments, the primer will be the same sequence as the primer binding site, as the primer will bind to a previously amplified product of the original primer binding site. In some embodiments, there may be a combination.

In addition, in some embodiments, at least a portion of the barcode can also be used a primer.

Cleavable Linker. The “cleavage domain” or “cleavable linker” represents link between at least two entities that can be used to reversibly attach said at least two entities. In some embodiments, the at least two entities are macromolecules. In some embodiments, at least one the of the entities is a substrate, or connected to a substrate.

In some embodiments, the cleavage domain linking the entities is a disulfide bond. A reducing agent can be added to break the disulfide bonds, resulting in the separation of the entities. As another example, heating can also result in degradation of the cleavage domain and separation of the entities. In some embodiments, laser radiation is used to heat and degrade cleavage domains, in some embodiment the laser radiation is targeted at specific locations. In some embodiments, the cleavage domain is a photo-sensitive chemical bond (e.g., a chemical bond that dissociates when exposed to light such as ultraviolet light).

Oligonucleotides with photo-sensitive chemical bonds (e.g., photo-cleavable linkers) have various advantages. They can be cleaved efficiently and rapidly (e.g., in nanoseconds and milliseconds). In some cases, photo-masks can be used such that only specific regions of the array are exposed to cleavable stimuli (e.g., exposure to UV light, exposure to light, exposure to heat induced by laser). When a photo-cleavable linker is used, the cleavable reaction is triggered by light, and can be highly selective to the linker and consequently biorthogonal. Typically, wavelength absorption for the photo cleavable linker is located in the near-UV range of the spectrum. In some embodiments, absorption maximum of the photo-cleavable linker is from about 200 nm to about 600 nm.

Non-limiting examples of a photo-sensitive chemical bond that can be used in a cleavage domain include those described in [Leriche, 2012] and [Weissleder, 2013, 2017/0275669], both of which are incorporated by reference herein in their entireties. For example, linkers that comprise photo-sensitive chemical bonds include 3-amino-3-(2-nitrophenyl)propionic acid (ANP), phenacyl ester derivatives, 8-quinolinyl benzenesulfonate, dicoumarin, 6-bromo-7-alkixycoumarin-4-ylmethoxycarbonyl, a bimane-based linker, and a bis-arylhydrazone based linker. In some embodiments, the photo-sensitive bond is part of a cleavable linker such as an ortho-nitrobenzyl (ONB) linker. Other examples of photo-sensitive chemical bonds that can be used in a cleavage domain include halogenated nucleosides such as bromodeoxyuridine (BrdU). Brdu is an analog of thymidine that can be readily incorporated into oligonucleotides, and is sensitive to UVB light (280-320 nm range). Upon exposure to UVB light, a photo-cleavage reaction occurs (e.g., at a nucleoside immediately 5′ to the site of Brdu incorporation ([Doddridge, 1998] and [Cook, 1999]) that results in cleavage of the cleavage domain.

Other examples of cleavage domains include labile chemical bonds such as, but not limited to, ester linkages (e.g., cleavable with an acid, a base, or hydroxylamine), a vicinal diol linkage (e.g., cleavable via sodium periodate), a Diels-Alder linkage (e.g., cleavable via heat), a sulfone linkage (e.g., cleavable via a base), a silyl ether linkage (e.g., cleavable via an acid), a glycosidic linkage (e.g., cleavable via an amylase), a peptide linkage (e.g., cleavable via a protease), an abasic or apurinic/apyrimidinic (AP) site (e.g., cleavable with an alkali or an AP endonuclease), or a phosphodiester linkage (e.g., cleavable via a nuclease (e.g., DNAase)).

In some embodiments, the cleavage domain includes a sequence that is recognized by one or more enzymes capable of cleaving a nucleic acid molecule, e.g., capable of breaking the phosphodiester linkage between two or more nucleotides. A bond can be cleavable via other nucleic acid molecule targeting enzymes, such as restriction enzymes (e.g., restriction endonucleases). For example, the cleavage domain can include a restriction endonuclease (restriction enzyme) recognition sequence. Restriction enzymes cut double-stranded or single stranded DNA at specific recognition nucleotide sequences known as restriction sites. In some embodiments, a rare-cutting restriction enzyme, e.g., enzymes with a long recognition site (at least 8 base pairs in length), is used to reduce the possibility of cleaving elsewhere.

In some embodiments, the cleavage domain includes a poly(U) sequence which can be cleaved by a mixture of Uracil DNA glycosylase (UDG) and the DNA glycosylase-lyase Endonuclease VIII, commercially known as the USER™ enzyme. Releasable entities can be available for reaction once released.

In some embodiments, the cleavage domain includes a nickase recognition site or sequence. Nickases are endonucleases which cleave only a single strand of a DNA duplex. Thus, the cleavage domain can include a nickase recognition site such that nicking of the site destabilizes the physical link between the entities, and results in them being separated.

In some embodiments the cleavage domain includes double strand nucleic acid such that two strands are not 100% complementary (for example, the number of mismatched base pairs can be one, two, or three base pairs). Such a mismatch is recognized, e.g., by the MutY and T7 endonuclease I enzymes, which results in cleavage of the nucleic acid molecule at the position of the mismatch.

Binding. “Binding”, “bound”, “bind” as used herein generally refers to a covalent or non-covalent interaction between two entities (referred to herein as “binding partners”, e.g., a substrate and an enzyme or an antibody and an epitope). Any chemical binding between two or more bodies is a bond, including but not limited to: covalent bonding, sigma bonding, pi ponding, ionic bonding, dipolar bonding, metallic bonding, intermolecular bonding, hydrogen bonding, Van der Waals bonding. As “binding” is a general term, the following are all examples of types of binding: “hybridization”, hydrogen-binding, minor-groove-binding, major-groove-binding, click-binding, affinity-binding, specific and non-specific binding.

Specific and Non-Specific Binding. As used herein, the terms “specifically binds” and “non-specifically binds” must be interpreted in the context for which these terms are used in the text. For example, a body may “specifically bind” to a nucleic acid molecule but have no significant preference or bias with respect the underlying sequence of said nucleic acid molecule over some genomic length scale and/or within some genomic region. As such, in the context of molecule's sequence, the body “non-specifically binds” to said nucleic acid molecule.

When in the context of binding between physically distinct molecules, “Specific binding” typically refers to interaction between two binding partners such that the binding partners bind to one another, but do not bind other molecules that may be present in the environment (e.g., in a biological sample, in tissue) at a significant or substantial level under a given set of conditions (e.g., physiological conditions).

Preferentially Binds (High Affinity). The term “preferentially binds” means that in comparison between at least two different binding sites (the sites can be on the same entity, or can be physically different entities), there is a non-zero probability of binding between a certain body and both sites, however conditions can exist in which the probability of binding of the certain body is preferable at one site over another.

Universal. In comparison to target sequence specific, the term “universal” when used in reference to a primer, or other nucleic acid molecules is intended to mean a nucleic acid having a sequence designed to universally hybridize against all desired targets within the context of the text (eg: all chromosomes, all genomes, all genes, etc) without substantial bias over a certain length scale. This can be done via a purposely designed sequence, or a combination of sequences. In some embodiments, all possible combinations of base pairs may be considered of a certain sequence length, or a random subset, or a non-random subset. Hexamer primers used with MDA amplification are an example of such universal primers. For the sake of clarity, the term “universal” typically refers to a plurality of sequences forming a set, however the singular form is often used to describe. For example, the phrase: “an entity A contains a barcode and a universal primer”, means that for a collection of As, all having the same barcode, all As are randomly, or specifically, assigned one of the primer sequences that make up the set of the universal primer.

Affinity Group. An “affinity group” is a molecule or molecular moiety which has a high affinity or preference for associating or binding with another specific or particular molecule or moiety, its “affinity partner”. The association or binding with another specific or particular molecule or moiety can be via a non-covalent interaction, such as hydrogen bonding, ionic forces, and van der Waals interactions. An affinity group can, for example, be biotin, which has a high affinity or preference to associate or bind to the protein avidin or streptavidin. An affinity group, for example, can also refer to avidin or streptavidin which has an affinity to biotin. Other examples of an affinity group and specific or particular molecule or moiety to which it binds or associates with include, but are not limited to, antibodies or antibody fragments and their respective antigens, such as digoxigenin and anti-digoxigenin antibodies, lectin, and carbohydrates (e.g., a sugar, a monosaccharide, a disaccharide, or a polysaccharide), and receptors and receptor ligands.

An affinity group may be capable of click chemistry reactions.

Any pair of affinity group and its specific or particular molecule or moiety to which it binds or associates with can have their roles reversed, for example, such that between a first molecule and a second molecule, in a first instance the first molecule is characterized as an affinity group for the second molecule, and in a second instance the second molecule is characterized as an affinity group for the first molecule.

Photolabile Protecting Groups. A “photolabile protecting group” is a reactive functional group that interacts with an affinity group, such that when the photolabile protecting group is exposed to a certain light, the result is an increase in the likelihood said affinity group will bind to its associated binding partner when compared to its previous protected status. Prior to such light exposure, the affinity group is commonly referred to as being “caged”.

Many approaches are known in the art for caged affinity groups and methods of making and using, such as those which protect an affinity group to reduce or eliminate the affinity the affinity group possesses for a particular target binding species. These approaches can be utilized to prevent binding of a member with undesired targets which are otherwise capable of binding with the member, or for other purposes such as controlling the time and location of binding. Additionally, a variety of approaches can be utilized to ensure that the affinity of the affinity group for the target binding species is not reduced, at least not substantially, after uncaging (as compared to the affinity possessed without the involvement of caging). For instance, a non-limiting example within the process of manufacturing polymer arrays through photolithography is the protection of otherwise reactive functional groups with photolabile protecting groups (e.g., McNPOC, NNPOC, NPPOC). These reactive functional groups are then activated for coupling with monomers within certain regions of the substrate through selective illumination, with the light possessing wavelength(s) capable of photolyzing the photolabile protecting groups and freeing the previously protected, or caged, hydroxyl groups. This approach of protecting affinity groups within a cage is certainly not limited to photolithographic synthesis of nucleic acid arrays, and many variations and adaptations of the concept are well known in the art for use with a variety of molecules, such as nucleic acids, amino acids, antibodies, etc. in a variety of approaches, chemistries, and applications.

Certain embodiments herein utilize this concept with respect to photoprotection of biotin moieties. Specifically, a biotin molecule (or variant or analog thereof) is modified or otherwise altered such that it possesses one or more photoactivatable protecting groups. These protecting groups serve to significantly reduce the binding affinity that the modified biotin molecule possesses for avidin (or variants or modified versions thereof, such as streptavidin) compared to the unmodified state of the biotin molecule. Some embodiments employ a photoactivatable protecting group such that appropriate illumination removes the protecting group to uncage the biotin and restore its natural binding affinity for the appropriate avidin molecule at issue. As a non-limiting example, certain embodiments will utilize protective caging groups that subject to photolysis by illumination in the ultraviolet spectrum (e.g., illumination containing a wavelength of 365 nm).

Alternative embodiments employing protected biotin are also possible. For instance, if avidin is employed to capture a biotin associated target, such capture can be prevented while the biotin molecules are still protected within their cages. Selective removal of the cages to unprotect the biotin at the desired time, location, etc. allows capture of the biotin associated target by the avidin. A non-limiting example would be the use of avidin immobilized on a support to capture biotinylated antibodies, nucleic acids, or proteins.

Photoprotection of a molecule, such as biotin, is generally achieved through modification of the molecule with a photoactivatable protecting group, with the protecting group located at a critical position (e.g., deactivating a particular bond) to prevent undesired reactions while the molecule is still caged by the protecting group. The inactive, caged molecule is then uncaged through appropriate irradiation, such as illumination at one or more appropriate wavelengths. A common example of such illumination is ultraviolet light. For embodiments where the protected molecule is associated with molecules that might be damaged by shorter wavelengths within the ultraviolet spectrum (e.g., potential damage to DNA by using illumination with wavelengths shorter than 340 nm), longer wavelengths are more appropriate (e.g., 350 nm, 360 nm, 365 nm, 375 nm, 390 nm). For additional background material, see [Lusic, 2006], “A New Photocaging Group for Aromatic N-Heterocycles,” Synthesis, 2006, No. 13, pp 2147-2150 and [Lusic, 2007], “Photochemical DNA Activation,” Organic Letters, 2007, Vol. 9, No. 10, 1903-1905, which describe nucleobase caging with 6-nitropiperonyloxymethyl (NPOM) groups, and which are incorporated herein by reference in their entireties for all purposes.

Many approaches are available for the caging of polymers such as oligonucleotides with photolabile protecting groups. For example, the caging protecting group may be placed on internucleotide phosphates, various positions on the sugar, or the nucleobase. Certain approaches incorporate biotin during phosphoramidite synthesis of the oligonucleotides. For background regarding the use of biotin, particularly caged protected biotin, see U.S. Pat. Nos. [Barrett, 1989, U.S. Pat. No. 5,252,743]; [Barrett, 1989, U.S. Pat. No. 5,451,683]; [Fodor, 1989, U.S. Pat. No. 6,919,211]; and [Fodor, 1989, U.S. Pat. No. 6,955,915]; U.S. Patent Application Publication No. [Fodor, 1989, 2003/0119011]; and [Pirrung, 1996], all of which are incorporated herein by reference in their entireties for all purposes.

Primer. A “primer” is a single-stranded nucleic acid sequence having a 3′ end that can be used as a chemical substrate for a nucleic acid polymerase in a nucleic acid extension reaction. RNA primers are formed of RNA nucleotides, and are used in RNA synthesis, while DNA primers are formed of DNA nucleotides and used in DNA synthesis. Primers can also include both RNA nucleotides and DNA nucleotides (e.g., in a random or designed pattern). Primers can also include other natural or synthetic nucleotides described herein that can have additional functionality. In some examples, DNA primers can be used to prime RNA synthesis and vice versa (e.g., RNA primers can be used to prime DNA synthesis). Primers can vary in length. For example, primers can be about 6 bases to about 120 bases. For example, primers can include up to about 25 bases. In some cases, as when a primase is used, a primer may be as short as a single base.

Amplification. A “PCR amplification”, “PCR”, or “amplification” refers to the use of a polymerase to generate at least one copy of at least a portion of a nucleic acid molecule. Suitable reagents and conditions for implementing PCR are described, for example, in U.S. Pat. Nos. 4,683,202, 4,683,195, 4,800,159, 4,965,188, and 5,512,462, the entire contents of each of which are incorporated herein by reference. In a typical PCR amplification, the reaction mixture includes the genetic material to be amplified, an enzyme, one or more primers that are employed in a primer extension reaction, and reagents for the reaction. The oligonucleotide primers are of sufficient length to provide for hybridization to complementary genetic material under hybridization conditions. The length of the primers generally depends on the length of the amplification domains, but will typically be at least 4 bases, at least 5 bases, at least 6 bases, at least 8 bases, at least 9 bases, at least 10 base pairs (bp), at least 11 bp, at least 12 bp, at least 13 bp, at least 14 bp, at least 15 bp, at least 16 bp, at least 17 bp, at least 18 bp, at least 19 bp, at least 20 bp, at least 25 bp, at least 30 bp, at least 35 bp, and can be as long as 40 bp or longer, where the length of the primers will generally range from 18 to 50 bp. The genetic material can be contacted with a single primer or a set of two primers (forward and reverse primers), depending upon whether primer extension, linear or exponential amplification of the genetic material is desired.

In some embodiments, the PCR amplification process uses a DNA polymerase enzyme. The DNA polymerase activity can be provided by one or more distinct DNA polymerase enzymes. In certain embodiments, the DNA polymerase enzyme is from a bacterium, e.g., the DNA polymerase enzyme is a bacterial DNA polymerase enzyme. For instance, the DNA polymerase can be from a bacterium of the genus Escherichia, Bacillus, Thermophilus, or Pyrococcus.

The term “DNA polymerase” includes not only naturally-occurring enzymes but also all modified derivatives thereof, including also derivatives of naturally-occurring DNA polymerase enzymes. For instance, in some embodiments, the DNA polymerase can have been modified to remove 5′-3′ exonuclease activity. Sequence-modified derivatives or mutants of DNA polymerase enzymes that can be used include, but are not limited to, mutants that retain at least some of the functional, e.g., DNA polymerase activity of the wild-type sequence. Mutations can affect the activity profile of the enzymes, e.g., enhance or reduce the rate of polymerization, under different reaction conditions, e.g., temperature, template concentration, primer concentration, etc. Mutations or sequence-modifications can also affect the exonuclease activity and/or thermostability of the enzyme.

In some embodiments, PCR amplification can include reactions such as, but not limited to, a strand-displacement amplification reaction, MDA, MALBEC, a rolling circle amplification reaction, a ligase chain reaction, a transcription-mediated amplification reaction, an isothermal amplification reaction, and/or a loop-mediated amplification reaction.

In some embodiments, the amplification process is optimized for a single cell application. For reference, a variety of single-cell amplification techniques are reviewed here: [Yasen, 2020] [Huang, 2015].

In some embodiments, the primer is a universal sequence.

In some embodiments, the primer is attached to additional nucleotides that may not function as a primer, but may provide other functionality, such as a barcode.

Reversable Terminator Nucleotides. “Reversable terminator Nucleotides” are Nucleotide analogs that include terminators that reversibly prevent nucleotide incorporation at the 3′-end of the primer, however the terminator can be removed (‘reversable’), thus allowing the polymerase to continue nucleotide incorporation. One type of reversible terminator is a 3′-O-blocked reversible terminator. Here the terminator moiety is linked to the oxygen atom of the 3′-OH end of the 5-carbon sugar of a nucleotide. For example, U.S. Pat. Nos. [Benner, 2005, U.S. Pat. No. 7,544,794] and [Benner, 2009, U.S. Pat. No. 8,034,923] (the disclosures of these patents are incorporated by reference) describe reversible terminator dNTPs having the 3′-OH group replaced by a 3′-ONH2 group. Another type of reversible terminator is a 3′-unblocked reversible terminator, wherein the terminator moiety is linked to the nitrogenous base of a nucleotide. For example, U.S. Pat. No. [Efcavitch, 2013, U.S. Pat. No. 8,808,989] (the disclosure of which is incorporated by reference) discloses particular examples of base-modified reversible terminator nucleotides that may be used in connection with the methods described herein. Other reversible terminators that similarly can be used in connection with the methods described herein include those described in U.S. Pat. Nos. [Siddiqi, 2007, U.S. Pat. No. 7,956.,171], [Efcavitch, 2005, U.S. Pat. No. 8,071,755], [Stupi, 2011, U.S. Pat. No. 9,399,798], [Hutter, 2010], [Knapp, 2011], [Ju, 2006], [Wu, 2007], and [Drmanac, 2018, 2018/0223358] (the disclosures of these U.S. patents are incorporated by reference). For reviews of nucleotide analogs having terminators see e.g., [Chen, 2013] “The History and Advances of Reversible Terminators Used in New Generations of Sequencing Technology,” Genomics, Proteomics & Bioinformatics 11(1):34-40 (2013).

Reversable terminators can include a fluorescent dye, that may, or may not constitute part of the blocking mechanism. In other cases, the reversable terminators may not incorporate dye, but can be associated with a fluorescent signal via binding to a second body, such as the CoolMPS process described by [Drmanac, 2020]. Alternatively, the reversable terminator nucleotide may not be associated with a fluorescent signal, and is intended to be ‘dark’.

Any suitable reversible blocking group may be attached to a nucleotide to prevent further extension by the enzyme following the incorporation of a nucleotide into the synthesis strand in a given cycle and to limit incorporation into the synthesis strand to one nucleotide per step. In any the methods of the invention the reversible blocking group is preferably a reversible terminator group which acts to prevent further extension by a polymerase enzyme. Non-limiting examples of reversible terminators are provided by [Milton, 2018, Patent WO 2020/016606], and include: Propargyl reversible terminators, Allyl reversible terminators, Cyclooctene reversible terminators Cyanoethyl reversible terminators, Nitrobenzyl reversible terminators, Disulfide reversible terminators, Azidomethyl reversible terminators, and Aminoalkoxy reversible terminators. Nucleoside triphosphates with bulky groups attached to the base can serve as substitutes for a reversible terminator group on 3′-hydroxy group and can block further incorporation. This group can be deprotected by TCEP or DTT producing natural nucleotides.

Immobilized. As used herein, the term “immobilized” when used in reference to a molecules in direct or indirect attachment to a substrate via covalent or non-covalent bond(s). Indirect attached to the substrate may be via at least one additional intermediary molecule or body. In certain embodiments, covalent attachment can be used, but all that is required is that the molecules remain co-localized to the substrate under conditions in which it is intended to use. Non limiting examples include the entire molecule may be held stationary with respect to the substrate, or a portion of the molecule held stationary with respect to the substrate, while the remainder of the molecule has limited freedom of movement, or the molecule is indirectly attached to the substrate via an intermediary, and the entire molecule has some limited freedom of movement. For example, immobilization of an oligonucleotide to a substrate can occur via hybridization of said oligonucleotide to a secondary oligonucleotide, said secondary oligonucleotide at least partially containing a complementary sequence to the first, and itself immobilized to the substrate.

In certain embodiments, a molecule may be immobilized on a surface via physisorption.

In certain embodiments, molecules can include biomolecules, nucleic acid molecules, proteins, peptides, nucleotides, or any combination thereof.

Certain embodiments may make use of a substrate which has been functionalized, for example by application of a layer or coating of an intermediate material comprising reactive groups which permit covalent attachment to biomolecules, such as polynucleotides.

Exemplary bonding examples include click chemistry techniques, non-specific interactions (e.g. hydrogen bonding, ionic bonding, van der Waals interactions etc.) or specific interactions (e.g. affinity interactions, receptor-ligand interactions, antibody-epitope interactions, avidin-biotin interactions, streptavidin-biotin interactions, lectin-carbohydrate interactions, etc.). Exemplary bonding mechanism are set forth in U.S. Pat. Nos. [Pieken, 1998, U.S. Pat. No. 6,737,236]; [Kozlov, 2003, U.S. Pat. No. 7,259,258]; [Sharpless, 2002, U.S. Pat. No. 7,375,234] and [Pieken, 1998, U.S. Pat. No. 7,427,678]; and US Pat. Pub. No. [Smith, 2004, 2011/0059865], each of which is incorporated herein by reference.

Molecular Combing. Defined herein, “molecular combining” or “combing” refers to the process of immobilizing at least a portion of a macromolecule, in particular long nucleic acid molecules, to a substrate surface, or within a porous film on a substrate surface, such that at least a portion of the macromolecule is elongated in a plane that is substantially parallel to the surface of said substrate. The elongated portion can be fully immobilized to the substrate, or at least of portion of said portion have some degree of freedom. In some embodiments at least a portion of the molecule is elongated within a porous material film parallel to the surface of said substrate, or at least a portion of the molecule is elongated on top of a porous material film parallel to the surface of said substrate, or at least a portion of the molecule is elongated and suspended between two points.

In some embodiments, the substrate surface is at least part of a fluidic device.

In one embodiment, a single nucleic acid molecule binds by one or both extremities (or regions proximal to one or both extremity) to a modified surface (e.g., silanised glass) and are then substantially uniformly stretched and aligned by a receding air/water interface. Schurra and Bensimon (2009) “Combing genomic DNA for structure and functional studies.” Methods Mol. Biol. 464:71-90; See also U.S. Pat. No. [Bensimon, 1995, U.S. Pat. No. 7,122,647], both of which are herein incorporated by reference in their entirety.

The percentage of fully-stretched nucleic acid molecules depends on the length of the nucleic acid molecules and method used. Generally, the longer the nucleic acid molecules stretched on a surface, the easier it is to achieve a complete stretching. For example, according to Conti, et al., over 40% of a 10 kb DNA molecules could be routinely stretched with some conditions of capillary flow, while only 20% of a 4 kb molecules could be fully stretched using the same conditions. For shorter nucleic acid fragments, the stretching quality can be improved with the stronger flow induced by dropping coverslips onto the slides. However, this approach may shear longer nucleic acid fragments into shorter pieces and is therefore may not suitable for stretching longer molecules. See e.g., [Conti, 2003] Conti, et al. (2003) Current Protocols in Cytometry John Wiley & Sons, Inc. and [Gueroui, 2002] Gueroui, et al. (Apr. 30, 2002) “Observation by fluorescence microscopy of transcription on single combed DNA.” PNAS 99(9): 6005-6010, both of which are hereby incorporated by reference in their entirety. See also [Bensimon, 1994, U.S. Pat. No. 5,840,862], [Bensimon, 1995, WO 97/18326], [Bensimon, 1999, WO 00/73503], [Bensimon, 1995, U.S. Pat. No. 7,122,647] which are hereby incorporated by reference in their entirety. [Lebofsky, 2003] “Single DNA molecule analysis: applications of molecular combing.” Brief Funct. Genomic Proteomic 1:385-96, hereby incorporated by reference in its entirety.

In some embodiments, the long nucleic acid molecule is attached to a substrate at one end and is stretched by various weak forces (e.g., electric force, surface tension, or optical force). In this embodiment, one end of the nucleic acid molecule is first anchored to a surface. For example, the molecule can be attached to a hydrophobic surface (e.g., modified glass) by adsorption. The anchored nucleic acid molecules can be stretched by a receding meniscus, evaporation, or by nitrogen gas flow. See e.g., [Chan, 2006] “A simple DNA stretching method for fluorescence imaging of single DNA molecules.” Nucleic Acids Research 34(17): e1-e6, herein incorporated by reference in it entirety.

In the general methods described herein where-by one end of the molecule is bound to a surface during stretching, the nucleic acids can be stretched by a factor of 1.5 times the crystallographic length of the nucleic acid. Without being bound by a particular theory, the ends of the nucleic acid molecule are believed to be frayed (e.g., open and exposing polar groups) that bind to ionisable groups coating a modified substrate (e.g., silanized glass plate) at a pH below the pKa of the ionisable groups (e.g., ensuring they are charged enough to interact with the ends of the nucleic acid molecule). The rest of the double-strand nucleic acid molecule cannot form these interactions. As the meniscus retracts, surface retention creates a force that acts on the nucleic acid molecule to retain it in the liquid phase; however this force is inferior to the strength of the nucleic acid molecule's attachment; the result is that the nucleic acid molecule is stretched as it enters the air phase; as the force acts in the locality of the air/liquid phase, it is invariant to different lengths or conformations of the nucleic acid molecule in solution, so the nucleic acid molecule of any length will be stretched the same as the meniscus retracts. As this stretching is constant along the length of a nucleic acid molecule, distance along the strand can be related to base content.

Another embodiment, the nucleic acid molecule is stretched by dissolving the long nucleic acid molecules in a drop of buffer and running down the substrate. In a further embodiment, the long nucleic acid molecules are embedded in agarose, or other gel. The agarose comprising the nucleic acid is then melted and combed along the substrate.

In another embodiment, the molecule is attached to the substrate at least one specific point, allowing the remainder of the molecule a substantial amount of degree of freedom, such that portion of elongation in the molecule is obtained by the application of an an external force on the molecule in a direction that is substantially parallel to the surface of the substrate. Examples of such embodiments include “DNA curtains” [Gibb, 2012] where-by the point of attachment is a controlled process, or the point of attachment can be random via interactions of the molecule with fluidic features, for example pillars as shown by [Craighead, 2011, U.S. Pat. No. 9,926,552].

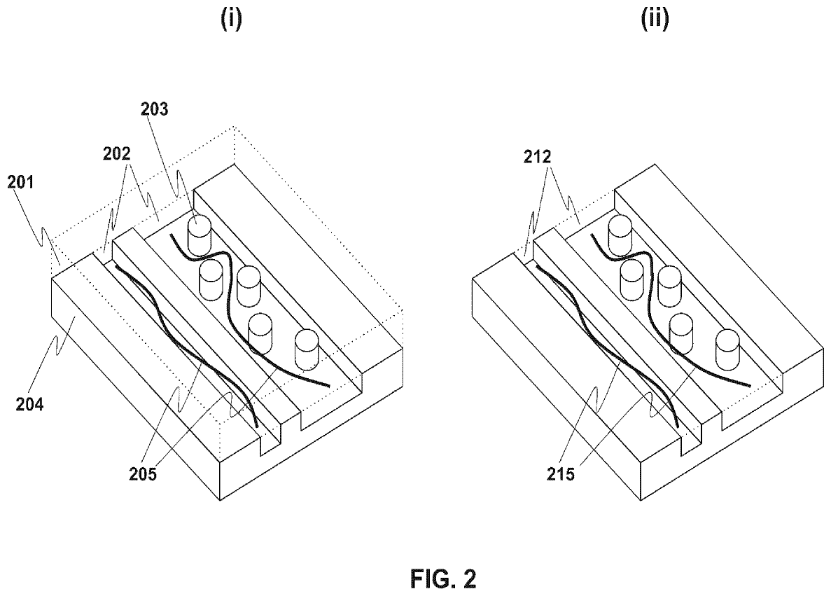

In some embodiments, molecular combing can be done with fluid flow generated by elongating the molecule in a fluidic device such that after elongation in the device, the molecule is presented in an elongated state on the surface of the device, or within a porous film on the surface of the device, In one embodiment, the molecule is elongated via an elongation channel that can elongate the molecule via methods described elsewhere in this disclosure, including confining dimensions, external force, interaction with physical obstacles, interaction with a functionalized surface, or combination there-of. In some embodiments, the fluidic channels of the device not fully confined, such that after evaporation of the transporting solution, the molecules are at least partially immobilized on the surface of the device in an elongated state. In some embodiments, as shown in in FIG. 2, a molecule 205 is elongated in a confined elongation channel of a microfluidic device (204), here with channel dimensions (202) that provide a confining environment and/or physical obstacles (203) that aid in promoting elongation. A gelling material within the solution that surrounds the molecule within the microfluidic device is then gelled. Finally, the molecules (215) are made accessible to the surface of the device via removal of the roof (201) while maintain the molecules within the gel film, or by using a porous roof material.

Microfluidic Device. The term “microfluidic device” or “fluidic device” as used herein generally refers to a device configured for fluid transport and/or transport of bodies through a fluid, and having a fluidic channel in which fluid can flow with at least one minimum dimension of no greater than about 100 microns. The minimum dimension can be any of length, width, height, radius, or cross-sectional axis. A microfluidic device can also include a plurality of fluidic channels. The dimension(s) of a given fluidic channel of a microfluidic device may vary depending, for example, on the particular configuration of the channel and/or channels and other features also included in the device.

Microfluidic devices described herein can also include any additional components that can, for example, aid in regulating fluid flow, such as a fluid flow regulator (e.g., a pump, a source of pressure, etc.), features that aid in preventing clogging of fluidic channels (e.g., funnel features in channels; reservoirs positioned between channels, reservoirs that provide fluids to fluidic channels, etc.) and/or removing debris from fluid streams, such as, for example, filters. Moreover, microfluidic devices may be configured as a fluidic chip that includes one or more reservoirs that supply fluids to an arrangement of microfluidic channels and also includes one or more reservoirs that receive fluids that have passed through the microfluidic device. In addition, microfluidic devices may be constructed of any suitable material(s), including polymer species and glass, or channels and cavities formed by multi-phase immiscible medium encapsulation. Microfluidic devices can contain a number of microchannels, valves, pumps, reactor, mixers and other components for producing the droplets. Microfluidic devices may contain active and/or passive sensors, electronic and/or magnetic devices, integrated optics, or functionalized surfaces. The physical substrates that define the microfluidic device channels can be solid or flexible, permeable or impermeable, or combinations there-of that can change with location and/or time. Microfluidic devices may be composed of materials that are at least partially transparent to at least one wavelength of light, and/or at least partially opaque to at least one wavelength of light.

A microfluidic device can be fully independent with all the necessary functionality to operate on the desired sample contained within. The operation may be completely passive, such as with the use of capillary pressure to manipulate fluid flows [Juncker, 2002], or may contain an internally power supply such as a battery. Alternatively, the fluidic device may operate with the assistance of an external device that can provide any combination of power, voltage, electrical current, magnetic field, pressure, vacuum, light, heat, cooling, sensing, imaging, digital communications, encapsulation, environmental conditions, etc. The external device maybe a mobile device such as a smart phone, or a larger desk-top device.

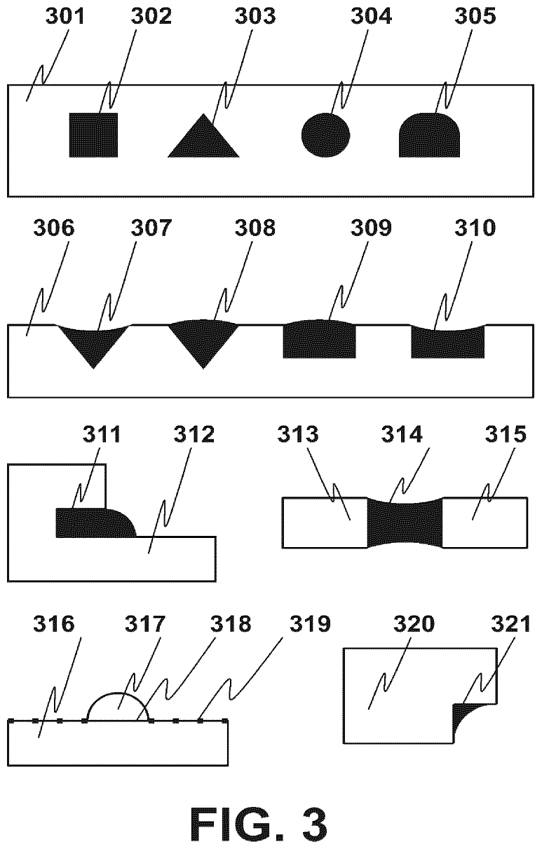

The containment of the fluid within a channel can be by any means in which the fluid can be maintained in a physical space within or on the fluidic device for a period of time. In most embodiments, the fluid is contained by the solid or semi-solid physical boundaries of the channel walls. FIG. 3 shows an example where-by channel walls with cross-sections such as rectangles (302), triangles (303), ovals (304), and mixed geometry (305) are all defined within a fluidic device (301). In other embodiments, fluidic containment within the fluidic device may be at least partially contained via solid physical boundaries in combination with surface energy changes and/or topological changes [Casavant, 2013], or an immiscible fluid [Li, 2020]. Examples of a fluid being at least partially confined within physical boundaries include various channels physically defined on the surface of a fluidic device (306) such as grooves (307, 308) and rectangles (309, 310), all of which are filled with liquid of sufficiently minimal quantity, that surface tension allows for the liquid to be physically maintained within the channels, and not overflow. In other embodiments, the channel (311) could be a defined by a groove in a U.S. (312) of a fluidic device, or the channel (314) could be defined by two physically separated boundaries (313 and 315) of a fluidic device, or the channel (321) could be defined by a U.S. (320) of a fluidic device. In other embodiments, the channel (317) is defined by a hydrophilic section (318) on the surface of a fluidic device (316) where-by the hydrophilic section is bounded by hydrophobic sections (319) on the surface of the fluidic device. In all cases, these embodiments are non-limiting examples.

It should be understood that some of the principles and design features described herein can be scaled to larger devices and systems including devices and systems employing channels and features reaching the millimeter or even centimeter scale channel cross-sections. Thus, when describing some devices and systems as “microfluidic,” it is intended that the description apply equally, in certain embodiments, to some larger scale devices. In addition, it should be understood that some of the principles and design features described herein can be scaled to smaller devices and systems including devices and systems employing channels and features that are 100s of nanometers, or even 10s of nanometers, or even single nanometers in scale channel cross-sections. Thus, when describing some devices and systems as “microfluidic,” it is intended that the description apply equally, in certain embodiments, to some smaller scale devices. As an example, a device may have input wells to accommodate liquid loading from a pipette that are millimeters in diameter, which are in fluidic connection with channels that are centimeters in length, 100 s of microns wide, and 100 s of nm deep, which are then in fluidic connection with nanopore constriction devices that are 0.1-10 nm in diameter.

A variety of materials and methods, according to certain aspects of the invention, can be used to form articles or components such as those described herein, e.g., channels such as microfluidic channels, chambers, etc. For example, various articles or components can be formed from solid materials, in which the channels can be formed via micromachining, film deposition processes such as spin coating and chemical vapor deposition, laser fabrication, photolithographic techniques, bonding techniques, deposition techniques, lamination techniques, molding techniques, etching methods including wet chemical or plasma processes, multi-phase immiscible medium encapsulation and the like. For patterning, a variety of methods may be employed, including but not limited to: photolithography, electron-beam lithography, nanoimprint lithography, AFM lithography, STM lithography, focused ion-beam lithography, stamping, embossing, molding, and dip pen lithography. For bonding, a variety of methods may be employed, including but not limited to: thermal bonding, adhesive bonding, surface activated bonding, fusion bonding, anodic bonding, plasma activated bonding, laser bonding, and ultra sonic bonding.

In one set of embodiments, various structures or components of the articles described herein can be formed of a polymer, for example, an elastomeric polymer such as polydimethylsiloxane (“PDMS”), polytetrafluoroethylene (“PTFE” or Teflon®), or the like. For instance, according to one embodiment, a microfluidic channel may be implemented by fabricating the fluidic system separately using PDMS or other soft lithography techniques [Xia, 1998, Whitesides, 2001].

Other examples of potentially suitable polymers include, but are not limited to, polyethylene terephthalate (PET), polyacrylate, polymethacrylate, polycarbonate, polystyrene, polyethylene, polypropylene, polyvinylchloride, cyclic olefin copolymer (COC), polytetrafluoroethylene, a fluorinated polymer, a silicone such as polydimethylsiloxane, polyvinylidene chloride, bis-benzocyclobutene (“BCB”), a polyimide, a fluorinated derivative of a polyimide, or the like. Combinations, copolymers, or blends involving polymers including those described above are also envisioned. The device may also be formed from composite materials, for example, a composite of a polymer and a semiconductor material. The device may be formed from glass, silicon, silicon nitride, silicon oxide, quartz. The device may be formed from a combination of different materials that are mixed, bonded, laminated, layered, joined, merged, or combination there-of.

Physical Obstacle. Unless specifically stated otherwise, a “physical obstacle” is a physical feature within a fluidic device in which a long nucleic acid molecule, in the presence of an applied force, physically interacts with, such that the molecule's physical conformation or location is different than had said physical obstacle not been present. Non-limiting examples include: pillars, comers, pits, traps, barriers, walls, bumps, constrictions, expansions. The physical obstacles need not be physically continuous with the fluidic channel, but may also be additive to the device, with non-limiting examples including: beads, gels, particles.

Droplet. The terms “droplet,” and “microdroplet” are used interchangeably herein, to refer to small, rounded structures (generally spherical in the unrestricted state), containing at least a first fluid phase, e.g., an aqueous phase (e.g., water), bounded by a second fluid phase (e.g., oil) which is immiscible with the first fluid phase, or bounded by surface tension formed by the interface of the first fluid phase, a surface, and air.

In some embodiments, droplets according to the present disclosure may contain a first fluid phase, e.g., oil, bounded by a second immiscible fluid phase, e.g. an aqueous phase fluid (e.g., water). In some embodiments, the second fluid phase will be an immiscible phase carrier fluid. Thus droplets according to the present disclosure may be provided as aqueous-in-oil emulsions or oil-in-aqueous emulsions. Droplets according to the present disclosure may be formed as multiple emulsions, such as double or higher level emulsions, for example resulting in aqueous-in-oil-in-aqueous droplets. In some embodiments, the subject droplets have a dimension, e.g., a diameter, of or about 0.1 μm to 1000 μm, inclusive. Furthermore, in some embodiments, discrete entities as described herein have a volume ranging from about 1 aL to 1 uL, inclusive droplets according to the present disclosure may be used to encapsulate cells, nucleic acids (e.g., DNA), enzymes, reagents, and a variety of other entities. The droplet may contain a single entity (eg: a single cell, or a single long nucleic acid fragment), or multiple entities. The droplet may contain a mixture of different types of entities. The term droplet may be used to refer to a droplet produced in, on, or by a microfluidic device and/or flowed from or applied by a microfluidic device. The droplet may be externally generated, and applied to the microfluidic device. Alternatively, the droplet may be generated within a microfluidic device, and then removed from said device.

Depending on the desired operation, droplets can be partitioned into 2 or more droplets, or merged with at least one other droplet. The droplet to be merged may have identical, or dissimilar contents.

The composition and nature of the droplets may vary. For instance, in certain aspects, a surfactant may be used to stabilize the droplets. Accordingly, a droplet may involve a surfactant stabilized emulsion. Any convenient surfactant that allows for the desired reactions to be performed in the droplets may be used. In other aspects, a droplet is not stabilized by surfactants or particles.

In some embodiments droplets may be formed by the interface of liquid, surface, and air, and thus include droplets defined by an electrowetting device. Examples of such droplets are reviewed by [Zhao, 2013] and [Mugele, 2005].

Encapsulation. Unless specifically stated otherwise, ‘Encapsulation’ refers to the point in time in which a body enters a droplet. This can occur at the moment of formation of a droplet, or later via the injection of the body into an existing droplet.

Entropic Barrier, Entropic Slopes, Entropic Traps, and Deformable Objects. A specific region of a nano or microfluidic device shall be defined as an “entropic barrier” if (a) the geometric shape of the device contains uneven features on the order of the size of the analyte of interest or less and (b) the diffusion or flow of the analyte around or through the features is significantly impeded or retarded in a manner that depends on the aggregate size, extended shape or conformation of the analyte. Furthermore, an “entropic trap” will be defined as region in a fluidic device where-by all fluidic connections are immediately through an entropic barrier such that if left at rest, the analyte of interest will remain in the trap, as the object occupying the trap is in a localized lowest energy state. The definition of Entropic Traps will be restricted to traps that are passive in nature in that they do not require a continuous supply of energy to hold an item or block its progression through a device, but do require energy to release it or cause it to pass through a barrier. The definition is further restricted to traps that are created by fabricating features such as pockets, constrictions, confinements, and physical obstacles into a fluidic device, and they can be partly or wholly defined by their geometry, and in turn lithographic artwork and processing parameters.

Entropic Traps allow for the spatial retention and positioning of packages, long polymer chain molecules and even subregions of long polymer chain molecules of interest. For brevity, all of these objects will be referred to as deformable objects in that their physical conformation can alter when in a confining fluidic device element, and they share many similarities governing their general behavior with respect to entropic traps and barriers. However, when their similarities diverge, or a particular feature of interest related to specific object is desired to reference, the specific object will be mentioned in the text.

Deformable objects can stay put in a trap, or against a barrier when buffer or surrounding fluid is flowed past at low velocity, permitting a change in chemical environment for reactions or the like. Further, traps can be designed to affect a change in the physical conformation of the deformable objects trapped within. While the geometry of various traps can look deceptively similar, their operating principles can vary significantly based on the size and composition of the trap and the deformable object to be trapped, as well as the chemical and local environment of the fluidic device that surrounds them. The methods of operation vary accordingly. The precise mechanisms of trap operation are a rich and ongoing area of physical investigation, but in most cases it is possible to enjoy the benefits of traps defined by geometry and method of use, without detailed understanding of the multiscale physical phenomena that underlie their use. Larger traps, such as traps for restraining intact cells or droplets, rely on elastic deformation of the object to be trapped, but are still referred to as Entropic Traps for consistency in this disclosure, as such objects similarly have a localized energy minimum when occupying a trap, and there exists a minimum applied force above which the deformable object can pass through any particular entropic barrier. This not entirely contrived; elasticity is a macroscopic manifestation of entropic forces amongst others. Likewise, in the context of an oil/water droplet system an Entropic Trap can be used to manipulate the motion and behavior of water droplets and is understood to be driven by minimization of surface tension energy.

Entropic Traps and barriers form a broad family of building blocks that can be arranged to create a fluidic device for the manipulation deformable objects. They are complementary to other building blocks such as channels, which move the deformable object and various reagents, manifolds which combine or split channels and interrogation regions which facilitate observation of the deformable object. They are also complementary to stationary phase materials as understood in the field of chromatography, which employ chemical attraction between the deformable object and the surface of a fluidic device or surface of a mechanically constrained accessory such as chromatography resin or bead, and function to retard the flow of the deformable object in mobile phase passing through a device. Entropic Traps and barriers are often found that the intersection of channels and/or interrogation areas and can be placed inside or adjacent to channels and interrogation areas, regions with defined surface chemistry or other building blocks. A specific part of a fluidic device can have qualities of an Entropic Trap or barrier and as well as qualities of another type of building block.

The confinement energy of an Entropic Trap is classically understood as the difference in free energy exhibited by a specific example, not meant to be limiting, of a long polymer as it occupies various physical conformations throughout the structure. A long polymer that undergoes random thermal motion in the presence of an entropic trap will move to the portion of the trap with the lowest free energy. Free energy has two parts, first a temperature-invariant enthalpic component such as the energy of a chemical state, stretched or constrained chemical bonds, electrostatic attraction or repulsion etc. Second there is an entropic component that lowers free energy in a manner proportional to both the temperature and the entropy of a long polymer in that part of the device, which is the number of ways in which the long polymer can conform inside the device. The analysis of Entropic Traps typically only considers the entropic component of free energy and neglects the enthalpic portion. Comparing two regions of an entropic trap, it is necessary to count the number of ways in which a random coiled polymer can occupy the trap. For example, a tight cylindrical pipe that is only slightly greater than the polymer's outer diameter will only allow a linear molecule to fit in two ways: forwards or backwards. In contrast, a large open volume will allow a combinatorial number of random runs, kinks and conformations. As there are more states in the latter case, the entropy of that geometry is higher, and the free energy of the long polymer in that area is correspondingly lower.

As the deformable objects move within a fluidic device to minimize free energy, they are said to fall into and occupy an Entropic Trap when they occupy a region of the device that allows for a localized lowest energy state. A deformable object that is entirely confined within a portion of a device with uniform geometry, but which does not extend into a neighboring trap will not spontaneously move into the trap, but will instead freely diffuse and move in response to external forces. However, when a portion of the deformable object diffuses into or is moved into region of the fluidic device which constitutes an entropic barrier from the reverse perspective, such fluidic element is an entropic slope, as the molecule will be drawn through the slope. Put in other terms, without an external force, a deformable object in certain physical conformation and location A within a fluidic device can lower its total energy by passing through an Entropic Slope to a new physical conformation and location B. However, the reverse is not possible without the addition of a minimum external applied force that allows the object to transfer through the entropic barrier from B to A.

A deformable object is freed from a trap when the difference in free energy from the trapped state to the liberated state is changed such that the free state now has a lower energy. For long polymers, this is typically accomplished by modulating the enthalpic portion of free energy by subjecting the molecule to an external force such as hydrodynamic drag from fluid flow or the application of an electric field to a molecule with net charge such as DNA.

The strength of the trap is understood in a probabilistic sense, in that the probability of escape from the trap decreases with increased trap energy. A well balanced trap will retain an item until displaced by means of an external force on the item, or by manipulation or modulation of the intrinsic trap itself.

The behavior of smaller traps that trap long polymers are influenced by, and can be modulated by, the chemical character of the long polymer, which can in turn be modulated by buffer conditions and the local chemical environment. On shorter length scales, the direction of extension of one segment of a polymer depends on the direction of the segments preceding it and is quantified by the intrinsic parameter known as the persistence length of the long polymer. A conformation that requires a long polymer to bend sharply relative to the persistence length will incur a spring energy. Self-avoidance dominates longer length scales, as when a polymer loops around it cannot overlap with prior segments. This loss of entropy is described by an excluded volume energy that is proportional to the molecule's diameter and net electrostatic charge.

In some embodiments, the deformable object is able to overcome entropic barriers at least partially due to a change in environmental conditions (for example temperature, pH, pressure) which act to reduce, or completely remove, the entropic barrier. For example, a long nucleic acid molecule can have it's radius of gyration altered by modifying the ionic concentration of the solution, thus allowing entropic barrier energy height to manipulated [Dai, 2016].

A long polymer chain (such as nucleic acid) left at rest in a solution will form a random coil configuration with outer boundaries that can be approximated as a sphere, and whose radius is governed by the properties of the solution and the molecule itself. This is the lowest energy state of polymer in a solution, and it will naturally return to this state if left unperturbed within the solution. However, when the polymer is in the presence of physical features and/or external forces that limit the polymer's ability to occupy a random coil conformation, the polymer chain will be physically manipulated into a higher energy state. Conversely, when physical boundaries and/or external forces are removed, the polymer chain will return to the spherical random coil configuration [Reisner, 2005][Han, 2007][Dai, 2016].

The interaction of long nucleic acid fragments with entropic traps and barriers in a fluidic environment was previously demonstrated [Craighead, 1999, U.S. Pat. No. 6,635,163]. Here the entropic barrier is an increase in physical confinement such that when a nucleic acid fragment transitions into the region of higher confinement, the nucleic acid's overall energy state increases. The amount of energy state change depends on the physical feature dimensions, the solution composition, and the polymer's physical properties. The energy increase provides a barrier, such that without a sufficiently large externally applied force, the long nucleic acid fragment will not move into the higher energy state. However, by applying a sufficiently large external force, the long nucleic acid molecule can be made to occupy the more confined region [Craighead, 1999, U.S. Pat. No. 6,635,163].

Similarly, a long nucleic acid molecule in an entropic trap will not escape, unless a sufficiently large external force is applied. Furthermore, a long nucleic acid fragment that is brought into physical contact with said trap, for example via an external force or Brownian motion, will relax into the trap. A long nucleic acid molecule will relax into the trap until its total energy state is minimized. As such, if the trap's physical dimensions are sufficiently small, only a portion of the long nucleic acid fragment may occupy the trap. This was demonstrated previously where-by small ‘pits’ (traps) were employed to capture sub-units of the long nucleic acid molecule in deformable objects composed of random coils in each trap, with the objects interconnected to each other by elongated portions of the molecule forming a “pearls on string” configuration [Reisner, 2009].

In addition to long nucleic acids molecules (long polymers), droplets (and in some cases cells) are also deformable objects that can be manipulated by entropic barriers, slopes, and traps. A droplet flowing in a channel will stop at a constriction (entropic barrier), and will not pass unless a sufficiently large force is applied on said drop (eg: pressure). Furthermore, a droplet can be trapped between two constriction points, and thus in an entropic trap, again until a sufficiently large external force (eg: pressure) is applied to release the droplet from the trap. [Tan, 2004[Fraden, 2007, U.S. Pat. No. 8,592,221] [Baroud, 2010]. Another example of an entropic trap is demonstrated by [Abbyad, 2011] in which droplets are “pinned” (“trapped”) along a rail, as the droplet has a localized lower energy state by relaxing into the rail structure.

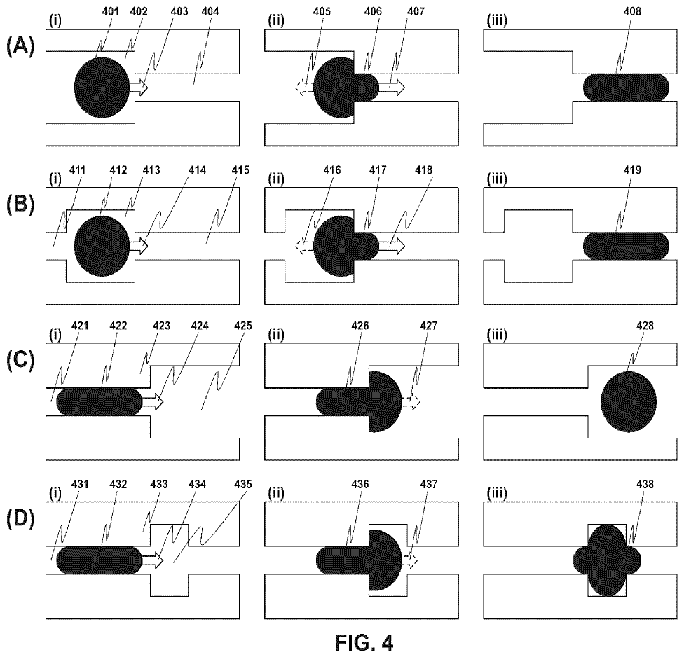

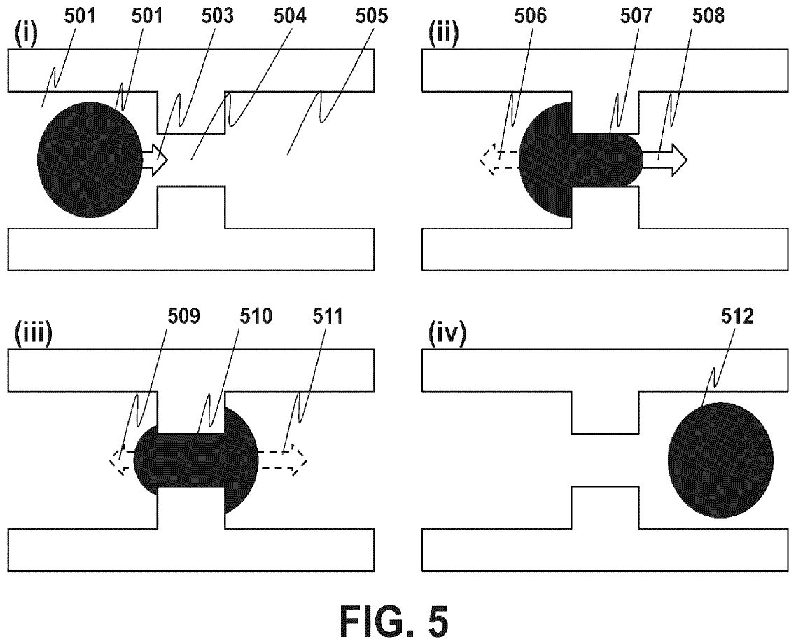

FIGS. 4 and 5 demonstrates some non-limiting examples of the interaction of entropic barriers, slopes, and traps with a deformable object when an external force is applied. All examples in FIGS. 4 and 5 are descriptive only, not wishing to be bound by any particular theory, and neglect secondary forces such as friction, Brownian motion, or pressure variation due to fluid displacement. In addition, the following examples described in FIGS. 4 and 5, an entropic barrier and/or slope are formed by the intersection of a wider channel with a narrower channel. In the Figures, the deformable object in its lowest energy conformation is described as a sphere which is reasonably accurate geometric approximation for a water-in-oil droplet. However for more complicated deformable objects that are composed of a non-homogenous material, long polymer chains, or structural asymmetries (eg: metaphase chromosomes), the lowest energy state conformation will be different. These non-limiting physical examples of an entropic barriers and slopes are purely for illustrative purposes and are meant to be simple demonstrations.

FIG. 4(A)(i, ii, iii) shows an example of a deformable object (401) in proximity to an entropic barrier, here identified as the intersection of the larger channel (402) with the narrower channel (404). With no external force applied, the object (401) will not enter the narrower channel (404), as doing so would require increasing the object's energy state. As such an external force (403) must be applied on the object otherwise it will remain within the larger channel (402). With an external force applied (407), the object will approach the entropic barrier and begin to deform (406) into a higher energy state. While the object is at least partially localized within the entropic barrier, a relaxing force (405) will pull the object back into larger channel. The magnitude of the relaxing force is dependent upon many factors, including the degree of deformation of the object, and how much of the object remains within the entropic barrier. If the external force (407) is sufficiently large to overcome the relaxing force (405), the object will overcome the entropic barrier. With no part of the object remaining within the barrier, the object to remain at rest at a higher energy state (408).

FIG. 4(B)(i, ii, iii) shows an example of a deformable object (412) in an entropic trap (413), in that all fluidic connections of the larger channel (413) are through one of two entropic barriers. The first entropic barrier being located at the interface of the larger channel (412) and narrower channel (415), and the second entropic barrier being located at the interface of the larger channel (412) and the narrower channel (411). With no external force applied, the object will remain in the trap indefinitely. However, by applying an external force (414), the object can be brought towards one of the entropic barriers, here the interface of 413 and 415. With an external force applied (418), the object will approach the narrow channel and begin to deform (417) into a higher energy state. While the object is at least partially localized within the entropic barrier, a relaxing force (416) will pull the object back into larger channel. The magnitude of the relaxing force is dependent upon many factors, including the degree of deformation of the object, and how much of the object remains within the entropic barrier. If the external force (418) is sufficiently large to overcome the relaxing force (416), then the object will overcome the entropic barrier, with no part of the object remaining within the barrier, allowing the object to remain at rest at a higher energy state (419).

FIG. 4(C)(i, ii, iii) shows an example of a deformable object (422) at rest in a deformed shape within a narrower channel (421), which is fluidically connected to a larger channel (425). The narrower channel (421) and larger channel (425) interface identifies an entropic slope with respect to the object's current state (422). An application of an external force (424) can bring the object into the presence of the entropic slope. When at least a part of the object has entered the slope, a relaxing force (427) will act to relax the object to lower energy state (426), moving the object into the larger channel. Once the object has exited the slope, the object will be at rest at a lower energy state (428) on the other side of the slope.

FIG. 4(D)(i, ii, iii) shows an example of a deformable object (432) at rest in a deformed shape within a narrow channel (431), which is fluidically connected to a larger channel (435). The narrower channel (431) and larger channel (435) interface identifies an entropic slope with respect to the object's current state (432). An application of an external force (434) can bring the object into the presence of the entropic slope. When at least a part of the object has entered the slope, a relaxing force (437) will act to relax the object to lower energy state (436), moving the object into the larger channel. In this example, the larger channel is insufficiently large to allow the object the freedom to completely relax to its lowest possible free energy state, however the final energy state of the object (438) is lower than the original state of the object (432), thus the object is now in an entropic trap (438).

FIG. 5 (i, ii, iii, iv) shows an example of a deformable object (501) in proximity to an entropic barrier, here identified as the intersection of the larger channel (501) with the narrower channel (504). With no external force applied, the object (501) will not enter the narrower channel (504), as doing so would require increasing the object's energy state. As such an external force (503) must be applied on the object otherwise it will remain within the larger channel (502). With an external force applied (508), the object will approach the entropic barrier and begin to deform (507) into a higher energy state. While the object is at least partially localized within the entropic barrier, a relaxing force (506) will pull the object back into larger channel. The magnitude of the relaxing force (506) is dependent upon many factors, including the degree of deformation of the object, and how much of the object remains within the entropic barrier. Provided the external force (508) is large enough to overcome the relaxing force (506), in this example the object is introduced to an entropic slope defined as the interface of the narrow channel (504) with the larger channel (505). Once in the presence of this entropic slope, an additional relaxing force (511) will act on the object in the direction of the larger channel (505). Again, the magnitude of the second relaxing force is a function of several parameters, including the physical position of the object within the slope. With the external force (508) still applied, at a certain point the secondary relaxing force (511) will overcome the first relaxing force (509), thus moving the object into the larger channel (512), with or without the external applied force.

For all embodiments, the physical confining dimensions of the entropic barrier and traps will be a function of the deformable objects in which the barrier and traps are designed to interact with. For example, a 300 nm nano-pit is appropriately sized to capture a 10 kbp segment of a 500 kbp long nucleic acid molecule, where-as a 20 micron constriction is appropriately sized to be a barrier for a 1 nL water-in-oil droplet.

Package. A “package” is any body capable of holding contents within the defined boundary of the body. In some embodiments, the boundary is defined by a physical barrier such as a lipid bilayer or a surfactant. In some embodiments there is no barrier, such as a droplet formed by mixing two immiscible fluids. A non-exhaustive list of packages include: cells, nucleus, vesicles, mitochondria, organelles, bacteria, virus, bubble, artificial membrane package, water-in-oil droplets, oil-in-water droplets, water-oil-water droplets, oil-water-oil droplets. In all cases, the package can be lysed (or ruptured) by various means to release the contents.

Porous Film. A “porous film” is any composition of solid, or semi-solid matter that is porous in nature. In some embodiments, it may be a gel, formed by cross-linking a gelling agent. In some embodiments, it may be an artificial gel, manufactured with either random, or controlled pore sizes. In some embodiments, it may be a material that is grown, etched, or deposited [Plawsky, 2009]. The material may be organic, inorganic, or a combination there-of. For the purpose of this document, the porous film should have pores of sufficiently small diameter that a portion of a nucleic acid molecule occupying said pores, can be maintained in an elongated state with no external force applied for a time duration long enough to allow for interrogation.

Gelling Agent and Gel. “Gels” are defined as a substantially dilute or porous system composed of a “gelling agent” that has been cross-linked (“gelled”). Non-limiting examples of gels include agarose, polyacrylamide, hydrogels [Caló, 2015], and DNA gels [Gačanin, 2020]. In the context of this document, a gel and a semi-gel are equivalent, where-by a semi-gel is a gel with incomplete cross-linking and/or low concentration of the gelling agent.

External Force. An “external force” is any applied force on a body such that the force that can perturb the body from a state of rest. Non-limiting examples include hydrodynamic drag exerted by a fluid flow [Larson, 1999] (which can be imitated by a pressure differential, gravity, capillary action, electro-osmotic), an electric field, electric-kinetic force, electrophoretic force, pulsed electrophoretic force, magnetic force, dielectric-force, centrifugal acceleration or combinations there-of. In addition, the external force may be applied indirectly, for example if bead is bound to the body, and then the bead is subjected to an external force such a magnetic field, or optical teasers.

Retarding Force. A “retarding force” is any force that retards a body's movement in the presence of an external force. Non-limiting examples include any of the following, or combination there-of: an entropic barrier, shear force, Van der Waals force, a physical obstruction, binding to surface (such as a substrate or bead), a gel, an artificial gel. It should be noted that the retarding force need not keep the body motionless, or maintain a zero-average velocity. In some cases, the retarding force may itself be an external force, such that two external forces counter-act each other, one acting to retard the body's movement in the direction of the first external force.

Photocleaving. “Photocleaving” Nucleic Acid is the process of introducing double stranded breaks in a Nucleic Acid molecule via the exposure of the molecule to a light source, possibly from the accumulation of multiple single strand breaks (nicks) in close proximity. In the preferred embodiment, a photosensitizer is used to transfer the energy from the photon to the molecule, as the molecule does not substantially absorb wavelength larger than 320 nm [Da Ros, 2005]and to avoid the accumulation of thymine dimers from UV exposure. The photosensitization could make use of oxygen and be of Type I or Type II as described in Baptista 2017. In the most preferred embodiment YOYO-1, or other members of the Cyanine dye family, are applied as an intercalator and excited at 488 nm in the absence of oxygen scavengers or radical scavengers [Akerman, 1996]. Unless specifically stated otherwise, ‘Photocleaving Nucleic Acid’ refers to the process of cleaving a double-strand Nucleic Acid molecule, preferably in the presence of a photosensitizer.

Dispensing System. Used herein, a “dispensing system” or “dispenser” is an instrument, or a component of an instrument that is capable of dispensing a volume of liquid from a dispensing tip, nozzle, or orifice (herein, collectively referred to as “tip”) at a desired location in (x, y, z) space. In some embodiments the liquid is dispensed as a continuous stream. In some embodiments, the liquid is dispensed as a series of drops. The drop size may be 100 micro liters or less, 10 micro liters or less, 1micro liters or less, 100 pico liters or less, 10 pico liters or less, 1 pico liters or less, 100 femto liters or less, 10 femto liters or less, 1 femto liter or less, 100 atto liters or less, 10 atto liters or less. In some embodiments, the tip is composed of a consumable pipette tip. In some embodiments, the dispenser tip is also capable of extracting solution from a target solution in (x, y, z) space, and so the dispenser is also an “extractor”. In some embodiments, the dispensing and extraction tips are different tips. In some embodiments, they are the same. In some embodiments, the tip is a micro-syringe, or the end of a capillary tube, or a nozzle. In some embodiments, the dispensing of liquid is controlled by air-displacement via a pressured air-line, or a syringe-pump moved via an electrical-mechanical system, such as a stepper motor.

In some embodiments, inkjet dispensers may be used. Inkjet printing includes continuous jet (CJ) and drop-on-demand jet (DODJ). The CJ based on the transducer, charging electrode and electric field can produce the droplet continuously, and the droplet location on a substrate can be determined by its charging density. There are several kinds of actuators for the DODJ device, including piezoelectric, thermal, solenoid, pneumatic, magnetostrictive and acoustic actuators. There are two actuation modes for the piezoelectric micro-jet devices in particular, including single actuation mode and hybrid actuation mode. The single actuation mode includes shear mode, squeeze mode, bend mode, push mode and needle-collision mode, while the hybrid actuation mode refers to electrohydrodynamic (EHD) assistant actuation. A detailed review of different inkjet technologies is provided by [Li, 2019], and included here for reference in its entirety.

In some embodiments the dispenser consists of a contact probe capable of transporting and depositing a drop of solution by contact wetting. In some embodiments, extraction of drop from a surface is done by a contact probe making contact with said drop, and wetting the contact probe.

Contact Probe System. Used herein, a “contact probe” system is an instrument, or a component within an instrument that is capable of positioning the point of a contact probe within the desired location in (x, y, z) space, preferably with nanometer position accuracy or better. In the preferred embodiments, the contact probe is capable of generating a signal based on its interaction with a physical object. In the preferred embodiments, the contract probe is a surface scanning probe, capable of generating a signal while the probe is physically moved in space by the instrument. Different types of probes include SPM (Scanning Probe Microscopy), AFM (Atomic Force Microscopy), STM (Scanning Tunneling Microscopy), SPE (Scanning Probe Electrochemistry). For a review of different Scanning Probe Microscopy systems, refer to [Takahashi, 2017]. In some embodiments, the contact probe can operate in a dry environment, or a humid environment, or a liquid environment. In some embodiments, the point of the contact probe can be functionalized with chemical moieties, biological bodies, or affinity groups to enable biochemical interaction with the physical object being probed. For a review of various functionalization that have been demonstrated on contact probes, refer to [Ebner, 2019]. In some embodiments, the point of the contact probe may include a carbon nanotube, a nanorod, or a nanospike.

### Targeted Manipulation of Regions of Interest (ROIs) of a Nucleic Acid Based on its Physical Map

The following disclosure, including set of embodiments of devices and methods allow for the targeted exposure of reagents and/or photons and/or contact probes to at least one ROI of a at least one long nucleic acid molecule. Through practice of the disclosure herein, the ROI(s) are at least partially identified by the analysis of the physical map on the molecule. The targeted exposure of regents, photons, or contact probe allows one to locally interact with the ROI, in some cases while it remains connected to the parent molecule. Optionally, the interaction involves enabling directly, or indirectly, an event such as a binding event, a reaction event, a cleavage event, or an enzymatic event within the ROL. In some cases all ROI are targeted. Alternately, not every ROI need be targeted for exposure. In some embodiments, ROI(s) are identified such that they inform the identification of additional ROI(s). In some embodiments, only a subset of ROI(s) are targeted. In some embodiments, a subset of ROI(s) from a first subset of molecules are used to identify an additional a subset of additional ROI(s) in a second subset of molecules. The first and second subsets of molecules can both each have an occupancy of at least one molecule, and the union of the first and second subsets can be zero or more molecules.

The ROI may be a single region along the length of a molecule such as a long nucleic acid molecule, or multiple regions. The ROI(s) may be each selected from separate criterion, or a combination of criterion. For example, one ROI on a long nucleic acid molecule may represent one gene, and a second ROI on the same molecule may represent a different gene. Optionally, a plurality of ROI(s) may represent a single higher-level ROI, for example, a series of ROI(s) that are all copies of the same genomic material, but located in different locations within a molecule such as a long nucleic acid molecule. An ROI may be defined as the boundary, neighbor, or flanking region of another ROI. The ROI(s) may be continuous along the molecule, discontinuous, or combination there-of. An ROI(s) may be defined in the negative, for example the non-ROI region(s). The ROI may constitute the long nucleic molecule in its entirety, or a majority there-of, or a portion down to a small portion of a molecule such as a nucleic acid molecule. In some embodiments, there may be at least 1, 2, 3, 5, 10, 25, 100, 500, 1000, 10000, 100000 or more ROI(s) within a long nucleic acid molecule. For embodiments where-by a long nucleic acid molecule constitutes a chromosome, or a large portion of a chromosome, ROI(s) could be all, or a subset-of-all, genes along the molecule, or all, or a subset-of-all, transcription factor binding sites, or all, or a subset-of-all regulatory regions. Other ROIs are also consistent with the disclosure herein.

The resolution of cleaving the ROI boundaries is in some cases impacted by the method of cleaving (enzymatic or photo energy, for example), the physical state of the parent when cleaving (in solution vs immobilized), or the resolution of the physical map generated to define the ROI(s) boundaries. Often, flanking material on either side of an ROI is included in order to account for resolution errors. In some embodiments, the flanking material may be at most, about or at least 1 bp in length, 10 bp in length, 100 bp in length, or at least 500 bp, or at least 1,000 bp, or at least 5,000 bp, or at least 10,000 bp.

The following subset of embodiments describe devices and methods for targeted exposure of reagents, photons, or a contact probe on at least one ROI within a long nucleic acid parent molecule at least partially based on analysis of the parent molecule's physical map.

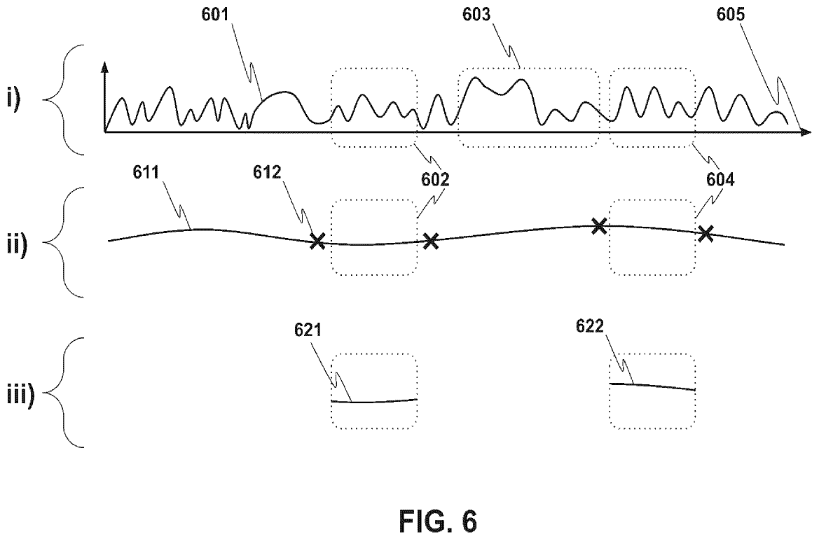

FIG. 6 demonstrates an embodiment where-by a long nucleic acid molecule 611 is interrogated in a fluidic device to generate a physical map 601 which provides information about the underlying genomic content of the molecule. The physical map in this embodiment expresses the relative AT/CG content ratio along the length 605 of the molecule, either in real length space, or in base-pair length space. If in base-pair length space, in the preferred embodiment, the conversion may account for variations in stretch, knots, confinement elongation, bodies binding to the molecule, and the molecule's underlying genomic content. The conversion can be as simple as multiplying measured contour length by a constant scaling factor or make more sophisticated use of computational mapping that permits local variation of the scaling factor as well as insertions and deletions. The conversion can further make use of integrated fluorescence along the DNA contours to estimate the density of DNA at each point [Perkins, 1995]. The ROI(s) are then selected based on an analysis of said physical map against a reference. For example, here ROI 602 is of interest, as the physical map pattern is identified as an insertion, while ROI 604 is of interest due to its close proximity to the region 603 in the physical map. After selection of the ROI(s), the ROI(s) in this embodiment are then removed from the parent molecule via a targeted cleaving at the desired ROI boundaries (612), which are themselves, also ROI(s). The separated ROI(s) 621 and 622 can then be collected.

Once the ROI(s) have been identified, any desired ROI may then be selectively exposed to reagents, photons, or contact probe, or any combination there-of.

The conditions under which an ROI is exposed to a reagent can vary from ROI to ROI. The exposure conditions that may vary include reagent concentration, reagent composition, reagent flow rate, reagent composition mixture ratios, and duration.

The conditions under which an ROI is exposed to photons can vary from ROI to ROI. The exposure conditions that may vary include wavelength, duration, intensity (brightness), polarization, angle of incidence.

The conditions under which an ROI is exposed to a contact probe can vary from ROI to ROI. The exposure conditions that may vary include contact probe type, contact probe point functionalization, contact probe operating mode, contact probe applied force.

For all embodiments (permitted by the device design), any ROI may be exposed to any combination of reagent exposure, photon exposure, and capture probe exposure. In addition, the conditions that may vary during such exposure include: temperature, ultrasonic power, application of external forces on the molecule (including on the ROI(s) and originating parent), the parent molecule and ROI's physical conformation and orientation, pressure, solution/rinse flow rate, humidity, buffer composition, and pH.

For ROI(s) that are separated from parent form a child, the ROI(s) may be pooled together, or maintain their physical separation from each other such that each ROI is traceable, or subsets of ROI(s) may be pooled together.

In some embodiments, unique barcodes are associated with the ROI(s) or subsets of ROI(s). The barcode can be the same for all ROI(s), but unique for the originating parent molecule, chromosome, cell, tissue, or patient. In some embodiments the barcode is known, in other embodiments it's randomly, or blindly assigned. The barcode may be associated to the ROI by binding to the ROI, either directly, or indirectly through an intermediary body. In the preferred embodiment, the barcode is attached directly or indirectly to a universal primer which then binds to the ROI. In some embodiments the unique barcode is associated with the ROI via physical confinement, for example within a shared droplet, or a shared entropic trap, or well. In some embodiments, the unique barcode is created from a unique combination of barcodes.

In some embodiments, where-by a universal primer is bound to the ROI, the universal primer includes a primer binding site that can be used for targeted PCR amplification. In some embodiments, the primer binding site is unique to each ROI, or subset of ROIs. In some embodiments, the primer binding site is identical for all ROIs. In some embodiments, the specified primer designed for the primer binding site is the complement of the primer binding site, or identical to, as the primer will bind to an amplified product of the original primer binding site. Or combination there-of.

In some embodiments where-by a particular reagent is desired to interrogate a single-strand portion of the double strand long nucleic acid molecule, the reagent solution includes a recombinase enzyme to form D-loop as described by [Chen, 2016] such that a localized, stable de-natured portion can be maintained.

### Targeted Exposure of ROI(s) Within a Parent Molecule to Reagents or Photons

In this set of embodiment device and methods, at least one ROI along a long nucleic acid fragment is selectively exposed to reagents, photons, or contact probe while maintaining the long nucleic acid molecule intact.

### Targeting ROIs in a Molecule in a Confined Fluidic Device

In the following set of embodiments, the long nucleic acid molecule in a confined fluidic device is interrogated to generate a physical map, identify ROI(S), and then target said ROI(s) with reagents or photons. As previously described, in preparation for, and during interrogation, the long nucleic molecule may be subjected to various fluidic device elements, external applied forces, and reagents to “prepare for interrogation”. In some embodiment devices and methods, the act of interrogating the molecule, identifying the ROI(s) on the molecule, targeting the ROI(s), and in some embodiments, then separating the ROI(s) are all performed while the molecule is in the same region of the fluidic device. In some embodiments, these steps may be performed at different regions of the device. For example, in one region of the device, the molecule may be interrogated such that ROI(s) are determined, and then in another region of the device, the ROI(s) may be re-identified on the molecule, and targeted with reagents or photons. Re-identification of the ROI(s) need not necessarily require re-interrogating the physical map. For example, provided the molecule's orientation is tracked, the previously identified ROI(s) may be determined within the parent molecule by length measurement alone, eg: one particular ROI is 10,000 bp long, starting 100,000 bp from the head of the molecule.

### Targeting Regents to ROIs.

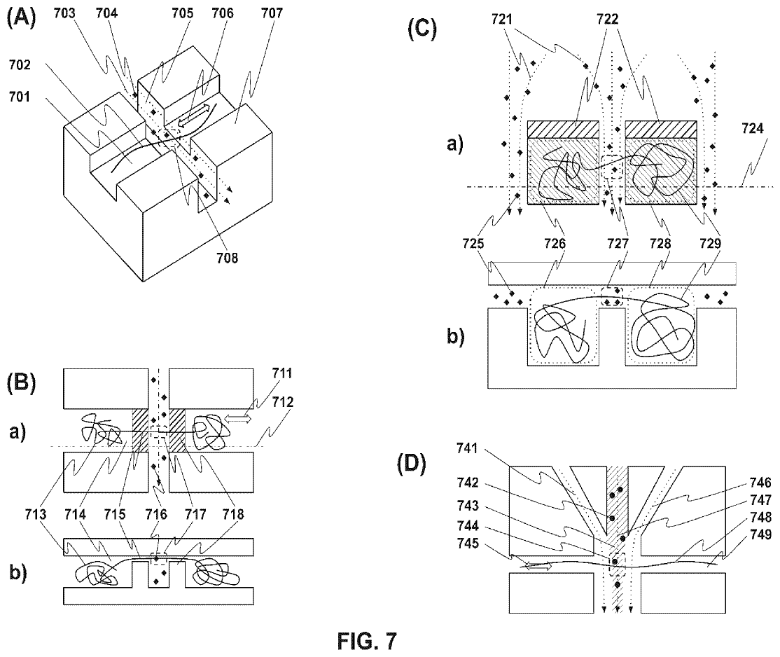

In this set of embodiment devices and methods, at least one ROI within a long nucleic acid molecule is targeted with reagents while said molecule is within a confined fluidic device. In the preferred embodiment, the ROI to be exposed is at least partially in an elongated state, enabling the ROI region to both be identified and targeted by the control system. FIG. 7(A) shows a confined fluidic device 707 (roof not shown) with a long nucleic acid molecule 702 in an elongated state, contained mostly within an elongation channel 701 with an ROI 708 exposed to a reagent at the intersection with a delivery cross-channel 705 that contains reagents 704. The reagents within the reagent delivery cross-channel are maintained within the cross-channel boundaries by laminar flow 703 in the cross-channel, and the molecule's physical position can be manipulated at least in part by the additional application of an applied force along the elongation channel 706.

In order to ensure that the reagent delivery channel does not substantially move the elongated long nucleic acid molecule during this process, the flow rate of the delivery channel needs to be balanced with a retarding force (eg: shear force) acting on the molecule in the elongation channel, preferably <1 μm/s local flow velocity. This can be accomplished via adjusting the dimensions of the elongation channel, and/or adding physical obstacles within the channel to increase the molecule's interaction with physical surfaces. In some embodiments, the depth of the reagent delivery channel may be different from that of the elongation channel. The width of the reagent delivery channel may be as wide or narrow as desired, with narrower channels providing respectively narrower regions of the molecule which can then be exposed, thus reducing the minimum ROI size that can be exposed.

In another embodiment of device and method there are physical obstacles within the intersection region that allow for physical support of the long molecule region exposed to the reagent flow. With such an embodiment, a physically larger ROI of the molecule can be exposed to reagents, without the molecule being pulled into the reagent delivery channel. In another embodiment of device and method, upstream of the reagent delivery channel may be a selection of different reagent sources, from which can be selected. In another embodiment of the device and method shown in FIG. 7(D), the reagent delivery channel has multiple laminar flows in parallel (741, 748, 746), which may have a combination of reagent components, including no active reagents in some or all flows. In the embodiment shown in FIG. 7(D), only the center flow (747) contains reagents (742), thus allowing for the adjacent laminar flows (741 and 746) to modulate the width of the reagent flow in the ROI region (744) by modulating the relative flow rates of the three laminar flows. In such a way, adjacent laminar flows can be used to ‘squeeze’ a particular laminar flow of reagents to the desired width. This width may be constant, or can vary depending on need of the application, A fluorescent dye can be added to the flow to aid in real-time calibration of the width. An externally applied force 745 can be applied to guide the ROI in the intersection for reagent exposure, and to remove from.

In another embodiment, two or more parallel laminar flows respectively carry different reagent compositions. Such a device is useful when the desired exposure composition is to change with time (example: expose the ROI to reagent composition A for 10 seconds, then reagent composition B for 5 seconds). By adjusting the widths of the laminar flows accordingly so their width ratios match the exposure time ratios, an ROI can then be transported through such an intersection. The long nucleic acid molecule need not be entirely elongated during selected exposure to a buffer flow, so long as the ROI can be moved into, and moved out, of the intersection region in a controlled manner, and there is a means to register the ROI in the intersection.

FIG. 7(B) shows an embodiment where-by the long nucleic acid molecule (713) is only elongated near the region of exposure (715), with the molecule originating from, and terminating at the outside of entropic barriers (718). In such a device, portion of molecule (717) being exposed to reagents (716) could be assessed by the quantity of nucleic acid molecule on either side via fluorescent intensity, or via recognition of the physical map present in the portion of molecule being elongated (715). Here, b) is a cross-section of a) through the line 712.

FIG. 7(C) shows one embodiment where-by a long nucleic acid molecule 729 is contained within two separate entropic traps 726 and 728, and the ROI is contained within the exposure region of the molecule that connects the traps 727. The ROI section of the molecule is then exposed to a flow (721) of solution containing at least one reagent (725). To protect the portion(s) of the molecule within the entropic trap(s) from exposure to said reagent, the laminar flow barriers can be used (722).). Here, b) is a cross-section of a) through line 724.

In another embodiment, the intersection of the two channels is physically large enough that the long nucleic acid molecule in the intersection can leave the elongated state and form a random coil within the region. In order to ensure containment of the long nucleic acid molecule within the intersection region, physical obstacles can be used, thus effectively turning the intersection region into an entropic trap. In this embodiment, portions of molecule elongation remain on either side of the intersection, thus these elongated molecule regions can be used for registration of the ROL Alternatively, the molecule can be loaded into the intersection region in a fully elongated state such that a physical map can be used to register an ROI, and then the ROI is allowed to relax into the entropic trap so that the molecule coils into a random coil within the entropic trap. The advantage of this embodiment is that a large section of molecule can be accommodated within the trap, with the amount of molecule determined by the trap's physical size.

For all embodiments, all possible combinations of relative movements of the nucleic acid molecule and reagent solution flow are possible, as different combinations may be useful for different applications. For example, if the desired ROI is larger than the regent delivery channel, the nucleic acid molecule can be manipulated by an external force to move the ROI through the intersection while the reagent solution flows, thus exposing variable length ROI along the long nucleic acid molecule to reagents. Alternatively, rather than a continuous movement of molecule through the intersection, a ‘step and repeat’ movement can also be used. In some embodiments, either the flow rate of the reagents, or the transport velocity of the long nucleic acid molecule, or both can be adjusted at anytime to impact effective exposure time along the length of the molecule to reagents. In some embodiments, the fluid flow rate in the reagent delivery channel is slowed down, or stopped, or reversed during ROI exposure. The ROIs to be exposed to reagents along the length of the long nucleic acid molecule need not be contiguous along the length of the molecule, but may be also be discontinuous. For all embodiments, there are a variety of methods that can be employed to move sections of the molecule through the intersection without exposing regions of the molecule to the reagents where not desired. The buffer composition in the reagent delivery channel can be alternated between a ‘neutral’ and an ‘active’ state, the later containing the reagents, and the molecule movement through the intersection timed accordingly as it moves through. Alternatively, there will be an exposure time window necessary for the reagent to interact with the molecule with statistical impact. As such, the sections of molecule that are not desired to be exposed can be moved through the intersection at a sufficiently fast speed that the probability of a reaction with the reagent is negligible for the application.

For all embodiments, the composition and rate flow of the buffer flow can change with time as desired such that a section of long nucleic acid molecule may be exposed to a series of different reagent solutions. Alternatively, the molecule can be moving while the composition changes such that a transition of different reagent exposures occurs along the length of the molecule. The flow rate may be adjusted to increase or decrease the probability of interaction of a particular reagent with the molecule. For all embodiments, there can be a multiple of elongation channels, and within a single elongation channel there can be multiple long nucleic acid molecules. Thus a single reaction delivery channel may intersect with multiple elongation channels. There can be multiple reagent delivery channels, with each flowing the same reagent, or combinations of reagents. Thus a single elongation channel may intersect with multiple reaction delivery channels. Within both the elongation channels, the reagent delivery channels, and their intersections, there can be physical obstacles. For all embodiments, methods of reagent flow control can include, but are not limited to: pressure, electro-kinetic, electro-osmotic, electro-phoretic, capillary.

In some applications it may be advantageous to selectively expose the tail end or loop-section of a long nucleic acid molecule to a desired reagent. Potential applications include the progressive exposure of a long nucleic acid molecule to a reagent, or the serial formation of child molecules from the long nucleic acid molecule via cleaving. In this embodiment device and method, a tail section or loop section of a long nucleic acid molecule can be exposed to a flow of reagents in a controlled manner. For all embodiments the length of the tail or loop being exposed can be controlled, preferably by fluorescent imaging of the long nucleic acid molecule. The length of the tail or loop section being exposed to reagents may remain static, grow, or retract. The composition of the reagents may be static, or fluctuate in time, including situations of no reagents. The solution flow rate that provides the reagents may also fluctuate in time. Thus, the flow rate of reagents, the composition of reagents (or lack there-of), and the length of the tail or loop being exposed to said reagents may all change with time. The coordination of these events may be controlled by a fluorescent imaging feedback system.

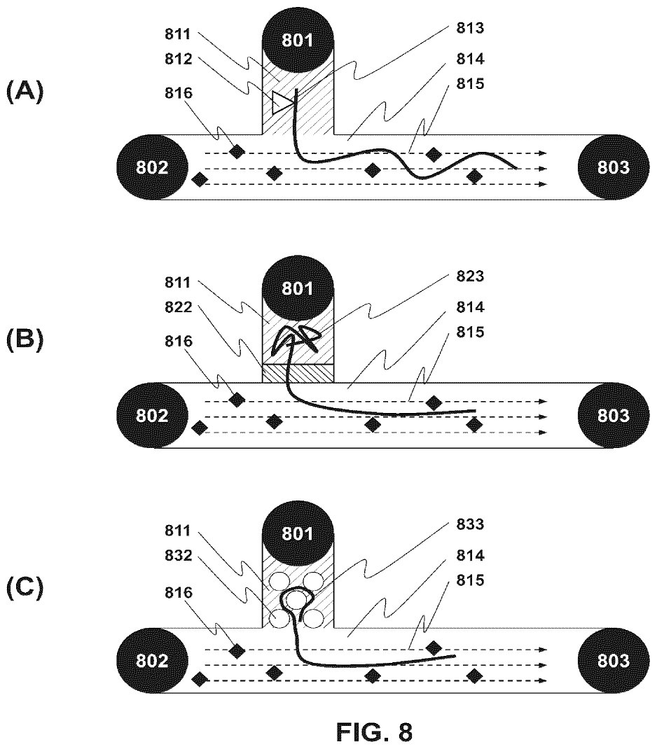

In one embodiment device and method shown in FIG. 8, a long nucleic acid molecule fragment 813 has a tail section exposed to a reagent buffer (816) in a reagent channel (814) flowing from (802) to (803). Here, the external force on the long nucleic acid molecule is the fluid flow (815) of the reagent buffer flow on the tail of the molecule. The retarding force (812) on the molecule maintains at least a portion of the molecule in a delivery channel 811 while the external force pulls the molecule taut. Should a portion of the tail break from the parent long nucleic acid molecule, forming a child molecule, either by design, or at random from stress, the remaining tail can be elongated via the application of an electro-kinetic force applied between (801) and (803). Furthermore, such an electro-kinetic force between could be used to retrack the tail from the reagent exposure if desired.

In other possible embodiments, the retarding force is an entropic barrier (822) interacting with a long nucleic acid molecule (823), or a collection of physical obstacles (832) interacting with a long nucleic acid molecule (833).

In another embodiment, a loop-section of the long nucleic acid molecule is exposed to the reagents, while the remainder of the molecule is excluded from the reagent exposure via a retarding force in a delivery channel.

In one particular embodiment, ROI(s) to be targeted along a long nucleic acid molecule are selectively exposed to universal primers, with here the reagent buffer flow containing universal primers, such as MDA primers. By flowing a universal primer reagent mix in an alkaline solution in the reagent delivery channel, the desired ROI(s) can be exposed to universal primers under conditions that allow for binding of the primers to the ROI(s). In some embodiments, the universal primers also include a barcode. In some embodiments, the universal primers also include a PCR sequence target that can then be used for targeted amplification with PCR primers after MDA with said universal primers.

### Targeting Photons to ROIs.

In this set of embodiment devices and methods, at least one ROI within a long nucleic acid molecule is targeted with photons while said molecule is within a confined fluidic device. In some embodiments, the ROI to be targeted is the boundary of another ROI, with the aim of targeting the photons on the long nucleic acid molecule to generate a cleave (break). In some embodiments the cleaving event is by photo-cleaving. In some embodiments, the targeting of the photons within the ROI is so that at least one cleaving event within the ROI is directly, or indirectly enabled, enhanced, activated, or modified by the photons. In some embodiments, the targeting of the photons within the ROI is so that at least one binding event within the ROI is directly, or indirectly enabled, enhanced, activated, or modified by the photons. In some embodiments, the targeting of the photons within the ROI is so that at least one enzymatic reaction event within the ROI is directly, or indirectly enabled, enhanced, activated, or modified by the photons. In some embodiments, a binding, cleaving, or enzymatic event within an ROI is directly, or indirectly enabled, enhanced, activated, or modified by de-protecting of an affinity group protected by a photoliable protected group. In some embodiments, a binding, cleaving, or enzymatic event within an ROI is directly, or indirectly enabled, enhanced, activated, or modified by photo-cleaving a photo-cleavable linker within a reagent.

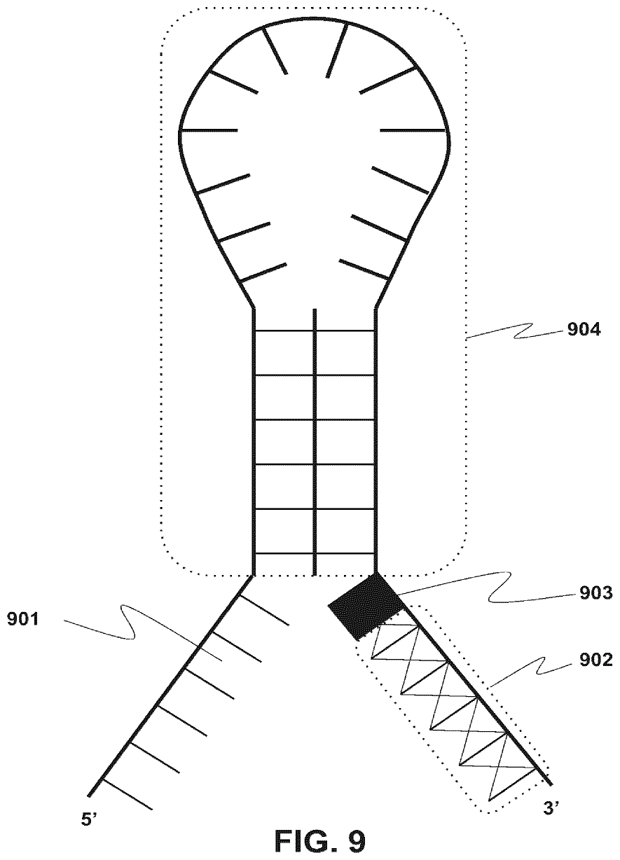

In some embodiments at least a portion of the parent molecule is exposed to captured primers, in which photons are used to facilitate the local release of universal primers around the ROI(s) allowing the released universal primers to bind to the ROI(s). Captured primers are defined as primers that are bound to a capture body that inhibits the primer from binding to a complementary nucleic acid strand, unless released from said capture body. In one embodiment shown in FIG. 9 a universal primer (902) is connected to a hairpin nucleic acid complex (904) through a cleavable linker (903), and extending on the other arm of the hairpin structure is a nucleic acid strand (901) that is non-complementary to the primer. In this state, the primer cannot have a complementary binding partner, as the primer is physically obstructed from doing do by the other non-complementary arm. However, once released from the hairpin structure via the cleavable linker, the primer is then free to hybridize to a complement. In the preferred embodiment, the cleavable linker is photo-cleavable. In the preferred embodiment the primer (902) is a universal primer. In some embodiments, the universal primers also include a barcode. In some embodiments, the universal primers also include a PCR sequence target that can then be used for targeted amplification with PCR primers after MDA with said universal primers.

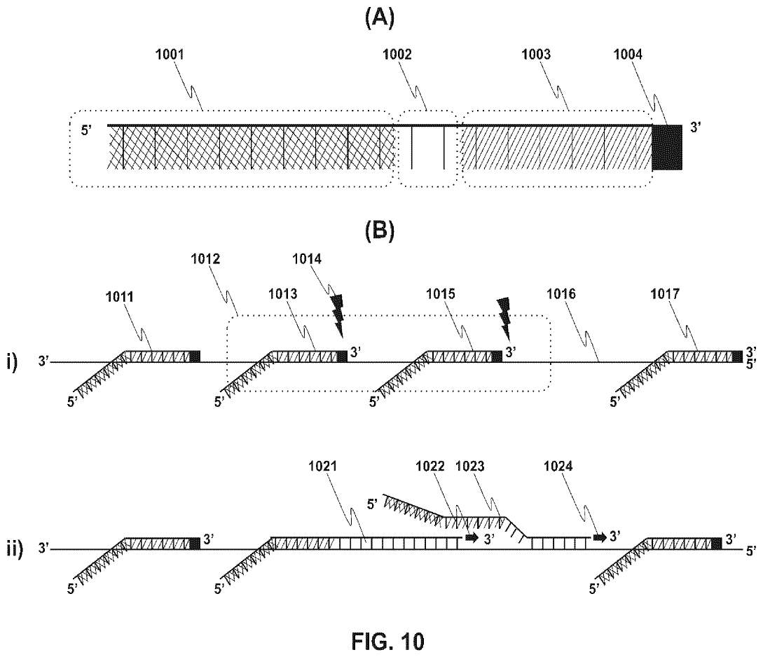

In some embodiments at least a portion of the parent molecule is exposed to non-active primers that bind to the exposed portion of the parent molecule, and a stimulus is used to facilitate the local activation of the primers in the ROI(s). In one embodiment shown in FIG. 10(A), the non-active primer consists of a reversable terminator nucleotide (1004) at the 3′ end, a universal primer segment (1003), an optional connection segment (1002), and then a barcode segment (1001). In some embodiments, the universal primer contains a 6-base (hexamer) sequence. In some embodiments, the last two bases of each barcoded primer contain thiophosphate modifications, which protect the primer from the 3′ exonuclease activity of Phi-29 nucleic acid polymerase. In the preferred embodiment, the barcode sequence is 8-24 bases in length. FIG. 10(B) demonstrates an embodiment method where-by non-active universal primers (1011, 1013, 1015, 1017) bind along the length of one strand of a long double stranded nucleic acid molecule (1016). The long nucleic acid molecule is at least partially, in at least one region, in a moment of time, in a state of de-nature such that the single strands in that region are available for hybridizing to said universal primers. The state of denaturation can be achieved by elevating the temperature, globally or locally, or changing the solution composition globally or locally, for example alkaline denaturation as is performed for MDA. Once bound, and ROI(s) regions have been identified (1012) along the long nucleic acid molecule, the ROI(s) are exposed to an appropriate wavelength of light (1014) that modifies the reversable terminator nucleotide, allowing for polymerase activity at the 3′ end of the primer to carry out primer extension (1022, 1024) and thus amplify the ROI(s) with complementary nucleic acid sequence (1021, 1023). In the preferred embodiment where-by MDA or MALBEC amplification is used, strand displacement will occur when polymerase activity of primer extension (1022) encounters another primer downstream.

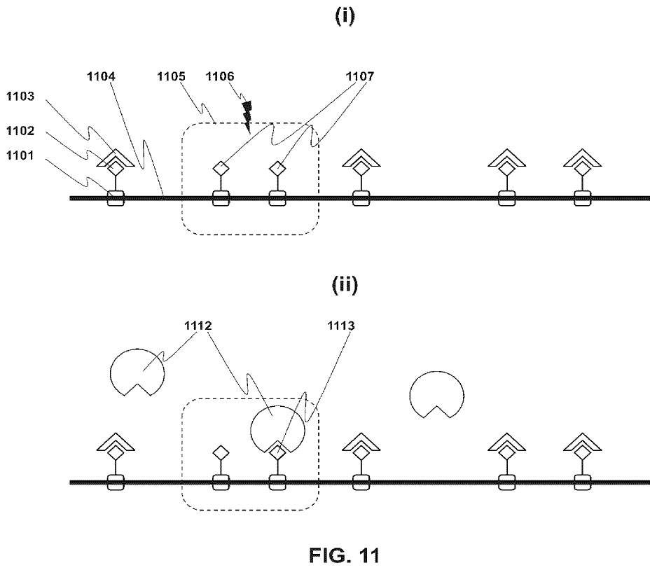

In some embodiments, a long nucleic acid molecule is bound to a plurality of bodies along the length of the molecule, with said bodies including a photo-liable protecting group that cage an affinity group, such that when exposed to the appropriate wavelength of light, an affinity group becomes un-caged. FIG. 11 demonstrates this embodiment, where-by a long nucleic molecule 1104 is bound to a plurality of bodies along the length of the molecule, where-by the bodies consists of a binding group 1101 that binds to the long nucleic acid molecule in a specific, or non-specific way, which is then connected to an affinity group 1102 which is caged when the photo-liable protecting group 1103 is blocking the affinity group. The ROI 1105 after exposure to the appropriate wavelength of light 1106, results in an un-caged affinity group 1107 that remains attached to the ROI region of the long nucleic acid molecule. After un-caging, the exposed affinity groups 1113 within the ROI are then available to bind to their affinity partner 1112.

The caged affinity group can be attached to the long nucleic acid molecule via a specific or non-specific binding group 1101. For example, the caged affinity may be attached to a long nucleic acid molecule via a hybridizing probe, a modified DNA binding protein, a modified DNA regulatory factor, modified DNA structural maintaining enzymes such as ATAC, a modified intercalator, a modified methyltransferase, a modified zinc finger, a modified recA, a modified restriction endonucleases, a modified CRISPR-CAS or any DNA/RNA editing enzyme complex with function knock-out, a modified transposase system such as Tn5, a modified telomerases, a modified retro-transposons.

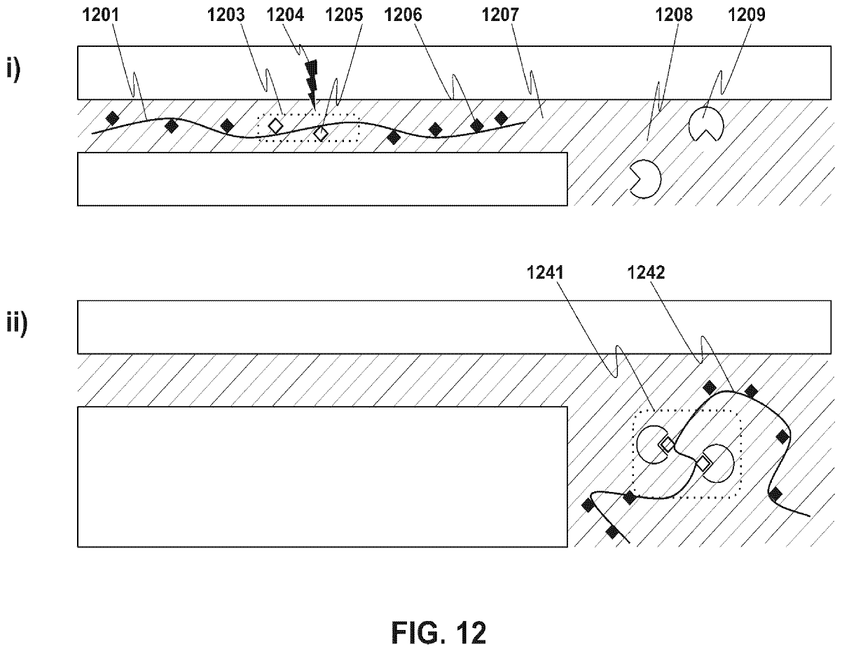

FIG. 12 demonstrates an embodiment of targeted ROI un-caging in a confined fluidic device. Here, a long nucleic acid molecule 1201 contained within an elongation channel (1207) of a confined fluidic device, has a plurality of bodies bound to it (1206) which contain a caged affinity group. Within an elongated portion of said molecule, a ROI is identified (1203), and within the ROI region the appropriate wavelength (1204) is used to un-cage the affinity group (1205). With the affinity group of the bound bodies within the ROI(s) now unprotected, the ROI(s) can now be bound to other bodies that contain the affinity group's binding partner (1209). In the preferred embodiment, the affinity group is a biotin, and the binding partner contains streptavidin. After bonding, the long nucleic acid molecule 1242 with its respective ROIs (1241) can then be further processed. In some embodiments, the binding partner (1209) contains a magnetic bead which then allow for the collection of said molecule with the application of a magnetic field or is a solid support such as a glass surface The use of other binding partner variations, such as avidin or streptavidin coated non-magnetic beads or other affinity systems such as digoxigenin: anti-dioxigenin or 2,4-dinitrophenyl (DNP): antiDNP would be obvious to those with skill in the art. Other examples of affinity groups that can be readily incorporated into oligos include click chemistry precursors such as azides, alkynes, vinyl and DBCO groups of said molecule with the application of a magnetic field or is a solid support such as a glass surface.

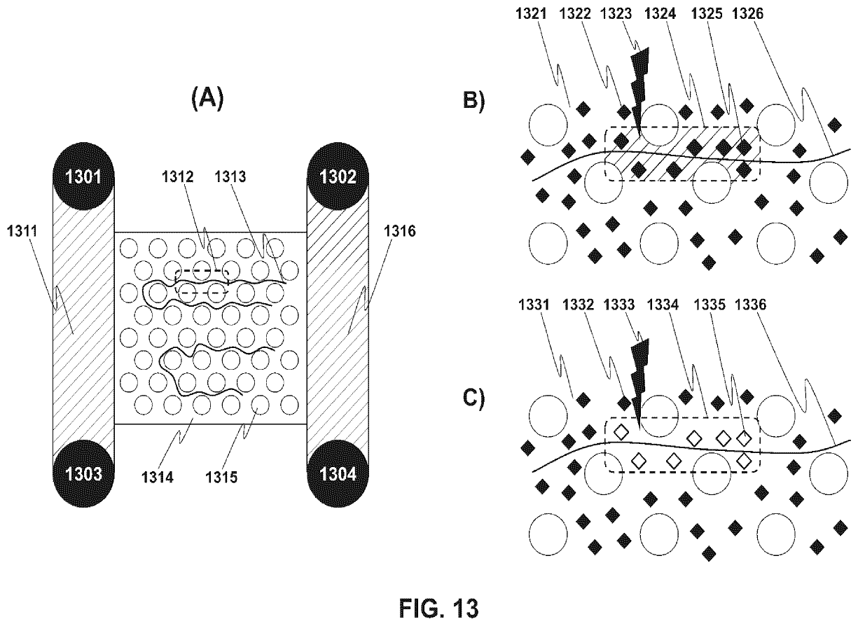

FIG. 13 demonstrates an embodiment where-by the reagents in close proximity to an ROI can be activated or have their reactivity modulated by targeted photon exposure. Here a solution containing the sample of at least one long nucleic acid molecule 1313 and agarose are flowed into an elongation channel (1314) that at least partially elongates the molecules. In this particular embodiment, the elongation channel is in fluidic connection with an inlet channel 1311 and an outlet channel 1316. In this particular embodiment, the elongation channel includes physical obstacles 1315 to promote elongation, although some embodiments may not have such physical obstacles. After loading the elongation channels with the long nucleic acid molecules surrounded by a solution containing agarose, the inlet and outlet channels are purged of the gel-containing solution, by displacing it with a non-gel-containing solution via the fluid connection ports (1301, 1303, 1302, 1304). Next the device temperature is lowered below the gel transition temperature such that the gel solution in the elongation channels solidifies (or semi-solidifies). With the long nucleic acid molecules in at least a partially elongated state such that the ROI(s) (1312) can be identified.

FIGS. 13(B) and (C) shows a zoomed in view of the ROI after gelling with two possible embodiments. In the embodiment shown in FIG. 13(B) the ROI region 1324 of the long nucleic acid molecule 1326 is exposed to IR photons 1323 so as to selectively melt region of gel around the ROL, and within the gel there are at least one type of reagent 1322. By melting the gel around the ROI, the reagents 1325 in close proximity to the ROI now have a higher mobility to diffuse and interact with their environment than similar reagents within the gel 1322. As such, the probability of the reagent participating in an enzymatic or binding event with the long nucleic acid molecule is higher within the ROI, than outside the ROI. In a further embodiment, the IR exposure can also at least partially de-nature the long nucleic acid molecule within the ROI, thus further increasing the probability of the reagent participating in an enzymatic or binding event, when such an event requires access to single-strand nucleic acid.

In the embodiment shown in FIG. 13(C), the ROI region 1334 of the long nucleic acid molecule 1336 is exposed to photons 1333 so as to selectively activate the reagents 1335 from their original unmodified form in the gel 1332. The activated reagents now have an increased probability of reacting with the nucleic acid in their proximity, either directly or indirectly. In some embodiments, a combination of both (C) and (B) are possible, such that the ROI is exposed to both to IR, and a different wavelength to activate the reagents.

### Targeting ROIs in a Molecule Immobilized on an Open Fluidic Device

In the following set of embodiments, the long nucleic acid molecule in an open fluidic device is interrogated to generate a physical map, identify ROI(s), and then target said ROI(s) with reagents, photons, or direct contact probing. Here, at least a portion of a long nucleic acid molecule is presented on the surface, or within a porous film on the surface, of an open fluidic device by combing said long nucleic acid molecule onto the open fluidic device, allowing for interrogating the molecule's physical map within the elongated portion of the molecule, identifying ROI(s), and then targeting said ROI(s) with reagents or photons.

In this set of embodiments, the ROI(s) to be targeted are on the surface of the open fluidic device, or are contained within a thin porous film on the surface of the device, or a combination there-of, and thus the ROI(s) are accessible to direct interaction with an applied solution, photons, or a contact probe. In the preferred set of embodiments, the process of interrogating the long nucleic acid molecule's physical map generates a coordinate map of the surface of the fluidic device in which the long nucleic acid molecules, their physical map, and their respective ROI(s) are located within said coordinate map. Employing such a map, the targeting of photons, dispensed solution, or a contact probe can be guided to the desired molecule or ROI on the surface. In the preferred embodiment, the open fluidic device physically engages with a control instrument that interrogates the long nucleic acid molecule's physical map, is the same instrument that directs the targeting of photons, dispensed solution, or contact probing. such that all electrical mechanical systems within the instrument can share the same coordinate space to target molecules and ROIs within the coordinate map. In some embodiments, the targeting is performed on a different instrument from the interrogation instrument, and fiducials on/within the open fluidic device are used to register the coordinate map,

### Targeting Dispensed Solution of Reagents to ROIs

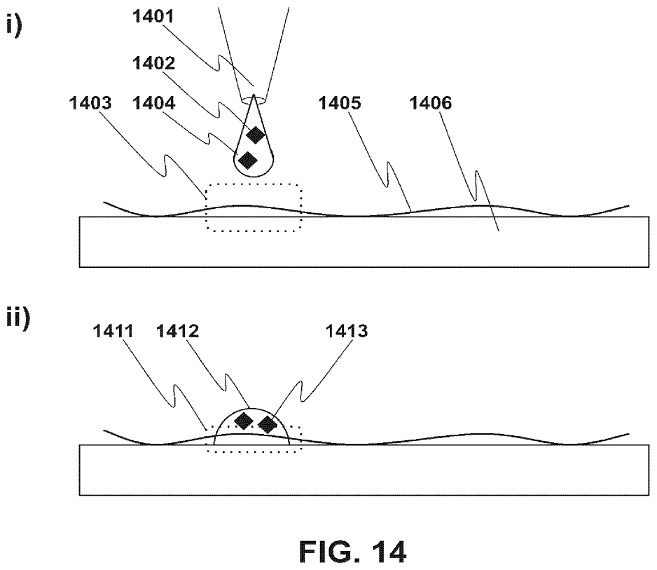

In this subset of embodiment devices and methods as shown in FIG. 14, identified ROI(s) (1403) contained within long nucleic acid molecule 1405 combed on the surface of the open fluidic device 1406 are targeted with a dispensed volume of liquid 1404 from a dispenser 1401. In the preferred embodiment, the liquid solution contains at least one reagent 1402 which can engage directly or indirectly in a binding or enzymatic reaction with the ROI. Once dispensed, the droplet 1412 containing the at least one reagent 1413 has sufficient volume of solution to submerge the ROI (1411), In some embodiments, an oil is then dispensed on the fluidic device, covering the drop, with said drop maintaining contact with the device surface and ROL

In some embodiments, the binding and/or enzymatic reaction takes place in the drop of solution that is dispensed with the reagent. In the preferred embodiment, the environmental conditions that the drop is contained in (humidity, temperature, pressure) while the drop is in contact with the ROI are controlled to minimize evaporation. In the preferred embodiment, the volume of reagent solution dispensed is controlled to minimize exposure of the solution to non-ROI regions. In some embodiments. the amount dispensed may a single drop of reagent solution, or multiple drops of reagent solution. In some embodiments, there may be multiple, different reagent solutions dispensed on a single ROI.

In some embodiments, the reagent solution(s) are allowed to dry on the ROI, such that the reagents are physically localized in proximity to the ROI. In some embodiments, at least one reaction involving at least one reagent occurs in a solution that is dispensed or applied without said reagent. For example, a series of different reagents may be dispensed on the ROI(s) and allowed to dry. After drying, another solution not containing reagents is dispensed or applied on the surface of the fluidic device over an area that substantially exceeds the ROI boundaries. In this embodiment, cross-talk of reagent interaction with non-ROI regions may be controlled by limiting the opportunity of diffusion of reagents to non-ROI regions via time limited process, after which the regents may be rinsed.

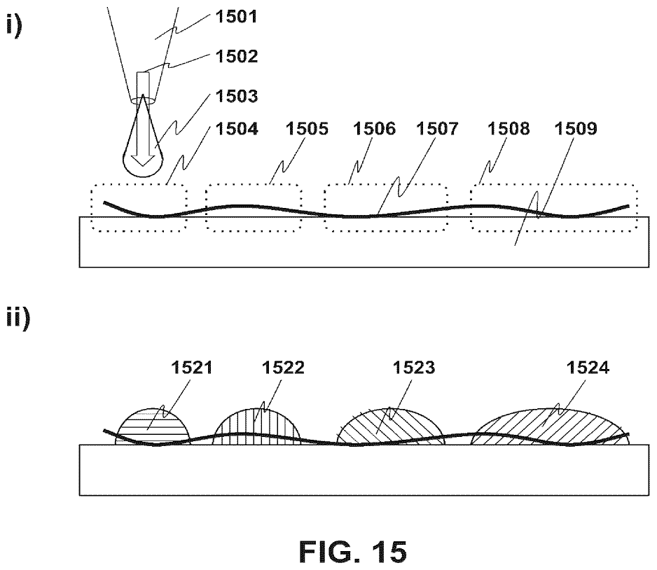

FIG. 15 describes an embodiment where-by each ROI along a long nucleic acid molecule (1507) combed on the surface of an open microfluidic device 1509 is exposed to desired reagent composition. Here shown is one dispenser 1501 capable of dispensing 1502 a solution containing at least one reagent 1503 on a desired ROI, and at least a second dispenser (not shown) capable of dispensing at least a second solution with at least a second type of reagent on a desired ROI. By adjusting combination of solution at dispensed at each ROI, in addition to the volume of each solution dispensed at each ROI, a desired reagent mixture can be selected for each ROI as desired. Thus, in FIG. 15 the ROI 1504 is exposed to reagent mixture 1521, the ROI 1505 is exposed to reagent mixture 1522, the ROI 1506 is exposed to reagent mixture 1523, and the ROI 1508 is exposed to reagent mixture 1524. In some embodiments, there may be 2 or more different solutions that can be independently dispensed. In some embodiments, 5 or more. In some embodiments 25 or more. In some embodiments 100 or more. In some embodiments, 1000 or more. In some embodiments, an oil is then dispensed on the fluidic device, covering the drop(s), which maintains contact with the device surface.

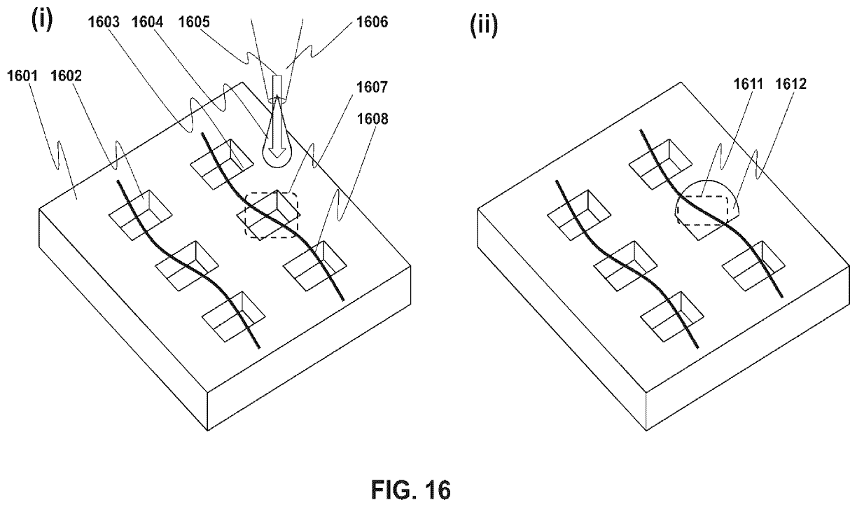

In some embodiments, the long nucleic acid molecules are combed on an open microfluidic device that includes patterning topological and/or surface energy modifications to form wells on the surface of the device, so as to physically contain the dispensed solution within said wells. FIG. 16 demonstrates an open fluidic device 1601 with fluid capture wells 1602 onto which long nucleic acid molecules are combed 1608 and then interrogated to identify ROI(s) 1607. A dispenser 1606 then dispenses 1605 a solution of at least one reagent 1603 onto the selected ROI, such that the selected ROI 1611 becomes submerged by the dispensed drop 1612 contained within the well. In the preferred embodiment, the size and density of patterning of patterning of the wells is such that the smallest ROI can be contained within a single well. In some embodiments an ROI may span multiple wells, requiring multiple dispensing events, at least one for each well. In the preferred embodiment, the surface of the wells are hydrophilic, while the regions between the wells is hydrophobic, such that a volume of liquid that can be dispensed into the well can exceeds the well's volume, while still being physically constrained by the well boundaries. In some embodiments, an oil is then dispensed on the fluidic device, covering the drop, with said drop maintaining contact with the ROI and the device surface inside the well.

In some embodiments, the bottom of the wells includes immobilized reagents on the surface of the well that can be re-suspended in the drop solution. In some embodiments, the reagents on the surface of the well are bound to the well surface by a cleavable linker, preferably a photocleavable linker. In some embodiments, the solution dispensed on the ROI contains no reagents, as the reagents originate from the well surface.

### Targeting Photons to ROIs.

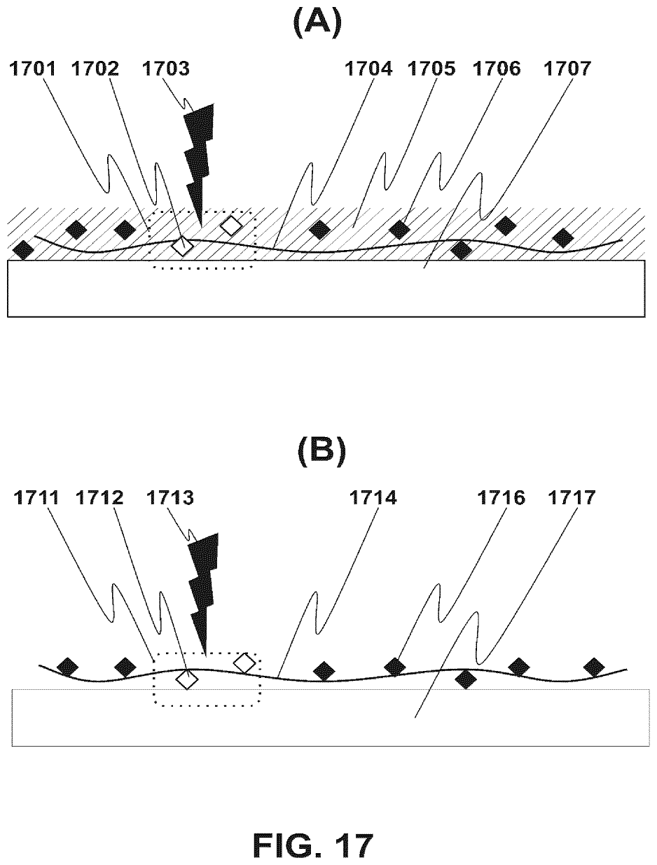

In this subset of embodiment devices and methods as shown in FIG. 17, identified ROI(s) within a long nucleic acid molecule combed on an open fluidic device are selectively exposed to photons. FIG. 17 demonstrates one embodiment where a long nucleic acid molecule 1704 that is combed on the surface of an open fluidic device 1707, such that at least a portion of the elongated molecule is contained within a thin film of porous material 1705. In this particular embodiment shown in FIG. 17(A), the porous material contains a photo-activated reagent 1706, which when exposed to the light, becomes activated 1702 to that it can engage in reactions directly or indirectly that results in a binding or enzymatic event within the ROI region.

In another embodiment, Figure AN(B) demonstrates a long nucleic acid molecule combed on the surface of an open fluidic device 1717, with said molecule bound to by a plurality of bodies 1716, each of which is includes a caged affinity group protected by a photo-liable protecting group. Upon exposure of an ROI 1711 to the appropriate wavelength of light 1713, the caged affinity groups within the ROI become un-caged, while still attached to long nucleic acid molecule, such that un-caged affinity groups are now free to bind to their respective affinity partners.

### Targeting Contact Probes to ROIs

In this subset of embodiment devices and methods, identified ROI(s) within a long nucleic acid molecule combed on an open fluidic device are selectively exposed to a contact probe. In some embodiments, the contact probe is functionalized such that the functionalized end of the contact probe can participate in a binding or enzymatic event with the nucleic acid within the ROI, either directly, or indirectly.

### Separation and Capture of ROI(s) From Parent Molecule

The fundamental goal of this series of embodiments is to provide some degree of control over the process of fragmentation of an originating (parent) long nucleic acid molecule into smaller child molecules such that knowledge is retained as to the positional origin of the child fragments within the parent molecule, and the children's relative position to each other. In some embodiments only information related to the relative order the children is maintained. In some embodiments, information related to both relative order, and relative distance apart (in bp) of the children is also maintained.

The originating nucleic acid molecule from which the smaller children are broken off (cleaved) from can include an entire chromosome, or a portion of a chromosome. The size of the children can range from 1 kbp to 1000 Mbp, depending on the needs of the application. In some embodiments the children are relatively equal in size. In some embodiments, the children vary in size. The size selection may be controlled, or random. In some embodiments the desired size can be selected on the fly.

The information about the child molecules can include, but is not limited to: the physical map of the child itself, the physical map of the parent molecule, or a portion of the physical map of the parent molecule around the region of fragmentation, the physical location of the child in reference to the parent molecule, any known information about the parent molecule (eg: originating cell, chromosome number, chromosome karyotype, cytogenetic information, disease type, etc) Depending on the device and method for cleaving the parent molecule, the location of fragmentation can be selected based on the physical map of the molecule, it's origin, the relative position of the child molecule along the length of the parent molecule, the identification of a known biomarker.

By measuring the physical distance of a long nucleic acid molecule with interrogation, the length of the parent molecule and any child molecule can be estimated, as a fully extended long nucleic acid molecule polymer is 0.34 nm/bp. By accounting for stretch variation due to conditions inherent in the interrogation of the molecule, a more accurate estimate of length can be determined.

Once the child molecules are generated, in some embodiments, at least one child molecule may then be physically segregated from the other children and positioning within an isolating region. In some embodiments, there is only one child per isolating region. In other embodiments, there is at least one child per isolating region. In some embodiments the isolating region is an entropic trap. In some embodiments, the isolating region is a droplet. In some embodiment the isolating region is a well. In some embodiments, each isolating region is then associated with a unique barcode, which may be known, or unknown.

### Parent Molecule in a Confined Fluidic Device

In the following set of embodiments, the long nucleic acid molecule is interrogated to generate a physical map, identify ROI(S), and separate the ROI(s) from the parent molecule in a confined fluidic device under the control of a control instrument. As previously described, in preparation for, and during interrogation, the long nucleic molecule may be subjected to various fluidic device elements, external applied forces, and reagents in order to “prepare for interrogation”. In some embodiment devices and methods, the act of interrogating the molecule, identifying the ROI(s) on the molecule, and then separating the ROI(s) are all performed while the molecule is in the same region of the fluidic device. In some embodiments, these steps may be performed at different regions of the device. For example, in one region of the device, the molecule may be interrogated such that ROI(s) are determined, and then in another region of the device, the ROI(s) may be re-identified on the molecule, and segmented from the parent molecule. Re-identification of the ROI(s) need not necessarily require re-interrogating the physical map. For example, provided the molecule's orientation is tracked, the previously identified ROI(s) may be determined within the parent molecule by length measurement alone, eg: one particular ROI is 10,000 bp long, starting 100,000 bp from the head of the molecule.

### Devices and Methods for Targeted Cleaving of Nucleic Acid Molecules in A Confined Fluidic Device

Generating separated ROI(s) from a long nucleic acid parent molecule requires the ability to cleave at ROI boundary(s), thus releasing the ROI and in doing so, creating a child molecule. Herein are a collection of non-limiting device and method embodiments for targeted cleaving of long nucleic acid parent molecules, which may be used individually, or in combination with each other.

### Generating Child Molecules by Cleaving a Long Nucleic Acid Molecule With Non-Rare Cutters

In this set embodiment devices and methods, a long parent nucleic acid molecule is fragmented into smaller children by the controlled exposure of an elongated portion of said molecules to a flow of non-rare cutters. This is done by selectively exposing the desired cleaving site (which then itself becomes an ROI) to a solution containing a non-specific nucleases or a nucleases whose recognition site is very likely (>90%) to be found within a relatively short span of nucleic acid, preferably <1 kbp, more preferably <100 bp.

Non-specific nucleases play very important roles in different aspects of basic genetic mechanisms, including their participation in mutation avoidance, nucleic acid repair, nucleic acid replication and recombination, scavenging of nucleotides and phosphates for the growth and metabolism, host defense against foreign nucleic acid molecules, programmed cell death and establishment of an infection. Due to their important roles in nucleic acid metabolisms, the sugar non-specific nucleases have been extensively used in molecular biology research, for example the determination of nucleic acid structure, the rapid sequencing of RNA, the removal of nucleic acids during protein purification and the use as antiviral agents. More than 30 nucleases have been obtained, such as staphylococcal nuclease, S. marcescens nuclease, SI nuclease, P1 nuclease, BAL 31 nuclease and NucA [Desai, 2003].

Using fluidic embodiments previously described for targeted exposure of an ROI to a reagent flow, a long nucleic acid molecule can have the desired cleaving sites (now themselves ROIs) exposed to a solution containing at least one nuclease in a controlled manner. In the preferred embodiment the long nucleic acid molecule is cleaved under tension, so that post-cleaving the tension on long nucleic acid molecule pulls both ends away from the solution containing the nucleases such that the probability of a second cut generating additional children is reduced.

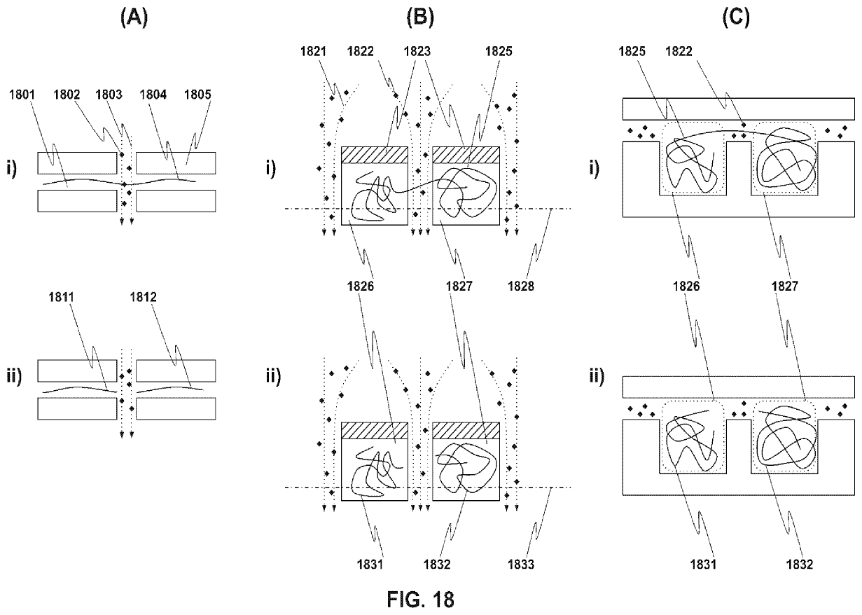

FIG. 18(A) shows one embodiment where-by an originating long nucleic acid molecule (1804) is maintained in an elongating channel 1801, while the region along the molecule where a desired cleave is located is exposed to a flowing cross channel (1803) of nucleases (1802). Once cut, two child molecules are then created (1811 and 1812).

FIGS. 18(B) and 18(C) shows one embodiment with FIG. 18(C)(i) showing the cross-section of FIG. 18(B)(i) at 1828, and FIG. 18(C)(ii) showing the cross-section of FIG. 18(B)(ii) at 1833, In this one embodiment, a long nucleic acid molecule 1825 partially occupying two separate entropic traps 1826 and 1827, forming deformable objects of coiled nucleic acid in each, with a section of the molecule spanning the two traps. The spanning section of the molecule is then exposed to a flow (1821) of solution containing at least one nuclease (1822). To protect the portion(s) of the molecule within the entropic trap(s) from exposure to said nuclease, the laminar flow barriers can be used (1823). After a single cleaving event along the spanning section of the molecule, two children long nucleic acid molecules are formed (1831 and 1832)

In all embodiments, a combination of different enzymes may be used.

### Generating Child Molecules by Cleaving a Long Nucleic Acid Molecule With Rare Cutters

In this set of embodiment devices and methods, a long parent nucleic acid molecule is fragmented into smaller children by the controlled exposure of an elongated portion of said molecules to a flow of rare cutters. A rare cutter is one who's recognition site is highly infrequent such that on average it will cut a target genome at a rate that will generate fragments of desired length, for example: on average every 100 kbp, or on average every 10 kbp, or on average every 1 kbp. Furthermore, the statistics around fragment lengths can be modified by using a combination of different rare cutters. Thus the choice of enzyme(s) will determine the distribution of fragment sizes.

In one embodiment demonstrated in FIG. 8, the tail (or loop section) a long nucleic acid molecule 813 is exposed to a flow (815) of solution that contains rare cutters (816), while the remainder of the molecule is maintained in the delivery channel (811), held within said channel by a retarding force (812). In this embodiment, the parent molecule enters the delivery channel from the fluidic connection 801, and the reagent solution flow is driven from fluidic connection port 802 to port 803, In some embodiments, the retarding force may be an entropic barrier (822) that interacts with the long nucleic acid molecule (823). In some embodiments, the retarding force may be physical obstacles (832) that interacts with the long nucleic acid molecule (833).

Once the tail is exposed to the rare cutter, provided a recognition site is present within the tail, a child molecule will separate from the parent molecule with the reagent flow, and may then be collected downstream at fluidic connection point 803.

By controlling the enzyme concentration, flow rates, and additional external forces, all operations can be done simultaneously such that as the tail is guided into the reagent channel, and as soon as a recognition site enters the reagent channel, it is cleaved, and a new child molecule is generated. Because the children are fragmented in a controlled, serial fashion, as they are released and flow down the reagent channel single-file, they can be collected in-order.

### Generating Child Molecules Via Photocleaving of a Long Nucleic Acid Molecule

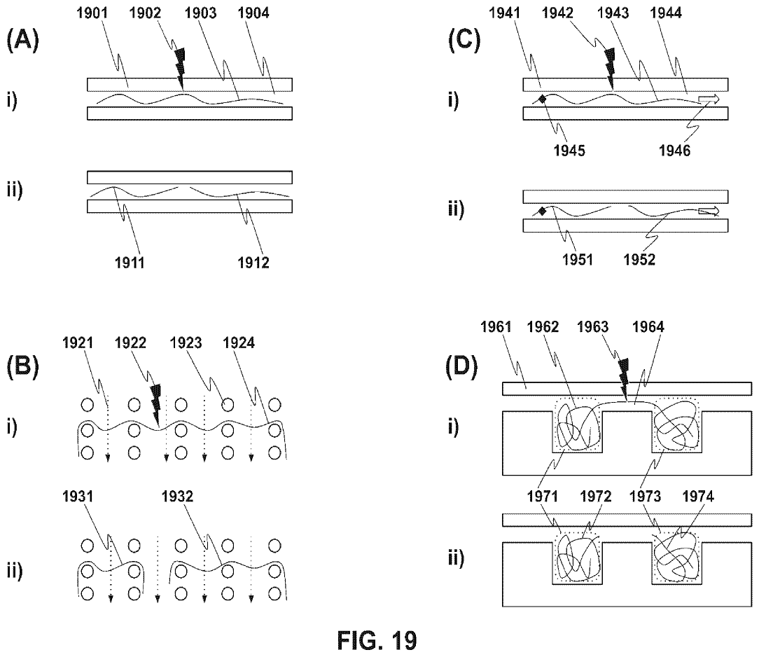

In this set of embodiment devices and methods, a long nucleic acid molecule in at least a partially elongated state is fragmented into children via photo-cleaving. FIG. 19 demonstrates a. collection of non-limiting embodiments where-by a long nucleic acid molecule can be selectively photo-cleaved at the desired cleaving site(s)(ROI(s)) by the targeted application of focused light of the appropriate wavelength. In all cases, ROI segmentation to generate a child from a parent is a generalization of the embodiments shown in FIG. 19, such that the targeted region(s) to be photo-cleaved are the boundaries of the child ROL

In the preferred embodiment, the region of the molecule being exposed to focused light for photo-cleaving is under a state of tension during the process such that post-cleaving, the two child molecules then retract from each other physically. Such physical separation post-cleaving reduces the likelihood of additional (unwanted) cleaving events from taking place, and enables methods of child separation and collection post-cleaving.

In 19(A) a long nucleic acid molecule (1901) is at least partially elongated within an elongation channel 1904 of a confining fluidic device (1901) such that a target site along the length of the elongated molecule can be identified, and light focused on to cleave. Upon photo-cleaving, two distinct child molecules are then generated 1911 and 1912. During the moment of photo-cleaving, the molecule may be at rest (except for Brownian motion), or may have at least one external force applied on the molecule. For ROI(s) generation, additional targeted photo-cleaves may be executed in a similar manner at the desired boundaries.

In 19(B) a long nucleic acid molecule (1924) is at least partially elongated within a fluidic chamber of a confining fluidic device containing physical obstacles 1923, while the molecule has an external force applied 1921, such that a target site along the length of the elongated molecule can be identified, and light focused on to cleave. Upon photo-cleaving, two distinct child molecules are then generated 1931 and 1932. For ROI(s) generation, additional photo-cleaves may be executed in a similar manner.

In 19(C) a long nucleic acid molecule (1943) is at least partially elongated within an elongation channel 1944 of a confining fluidic device (1941), while the molecule has at least one external force 1946, and at least one retarding force 1945 applied, such that a target site along the length of the elongated molecule can be identified, and light focused on to cleave. Upon photo-cleaving, two distinct child molecules are then generated 1951 and 1952. During the moment of photo-cleaving, the molecule may have no movement in center-of-mass, or the center-of-mass may be moving. For ROI(s) generation, additional photo-cleaves may be executed in a similar manner. This particular embodiment has the preferential benefit that after cleaving, the child fragment will physically separate from the parent via the application of the applied force, enabling a collection method of said child molecule.

In FIG. 19(D) a long nucleic acid molecule (1962) in a confined fluidic device 1961 is exposed to at least two entropic traps 1971 and 1973 separated by an elongation channel 1964 such that the molecule forms two distinct deformable objects of random coils in the entropic traps that are connected by a section of the molecule in the elongation channel. Upon focused exposure of light 1963, the two distinct child molecules form (1972, 1974), each contained in a respective entropic trap.

In some embodiments, not every interconnecting strand of nucleic acid molecule between entropic traps need be cleaved, but instead a sub-selection could be cleaved to generate a desired molecule size. For example, a long nucleic acid molecule occupying 5 entropic traps may be cleaved between traps 3 and 4, thus generating two child molecules, one of length that occupies traps 1, 2, and 3, and another that occupies traps 4 and 5.

In all embodiments, the efficiency of photocleaving nucleic acid can be improved by having photosensitizer present. The photosensitizer can be in the solution, bound to nucleic acid in some way, attached to the device, attached to a mobile body in some way.

In all embodiments, the physical resolution of cleaving can be improved by exposing the desired cleaving region of the long nucleic acid molecule to a concentrated region of photosensitizer. For example, a laminar flow of photosensitizers compressed by adjacent laminar flows that do not contain the photosensitizer, such that the width of such a laminar flow of photosensitizers is less than the wavelength of light used for photocleaving. In another example, the photosensitizers may be physically attached to the device, and the nucleic acid is brought into the vicinity of the photosensitizer where the desired cleave is to be made.

### Capturing Long Nucleic Acid Molecules in Confined Fluidic Devices

Here we disclose a series of embodiments that allow for the targeted capture long nucleic acid molecules in a confined fluidic device such that at least one long nucleic molecule can be positioned within any of at an at least one isolating region(s). In some embodiments, the isolating region is a droplet. In some embodiments, the isolating region is an entropic trap. In some embodiments, the isolating region is a container external to the device, for example a tube, a pipette tip, or a well.

In some embodiments, there is only one long nucleic acid molecule per isolating region. In some embodiments, there is at least one long nucleic acid molecule per isolating region. In all embodiments the nucleic acid molecules may be further processed and/or analyzed on-device, or removed from the device for further processing and/or analysis off device.

In some embodiments, the at least one captured long nucleic acid molecule is an ROI or child molecule from a parent long nucleic acid molecule.

### Capturing Long Nucleic Acid Molecules in Confined Fluidic Devices With Entropic Traps

In this set of embodiment devices and methods, ROI(s) are segregated from the parent long nucleic acid molecule by bringing the ROI(s) within proximity of an entropic trap. The ROI(s) can be identified before, or during this process of aligning the ROI(s) to the trap. The ROI(s) will then coil to fill the trap in an energetically favorable way. The amount of nucleic acid from the parent molecule that will occupy the trap will depend on the size of the trap and the composition and temperature of the solution in the confining fluidic device that surrounds the long nucleic acid molecule. As such, the physical size of the entropic trap can be defined to accommodate pre-determined sized ROI(s), or the device can be designed with several different sized traps to accommodate different sized ROI(s) as needed. Alternatively, in some embodiments, single ROI(s) may occupy at least one trap, such that the length of the ROI is defined by the number of traps it occupies.

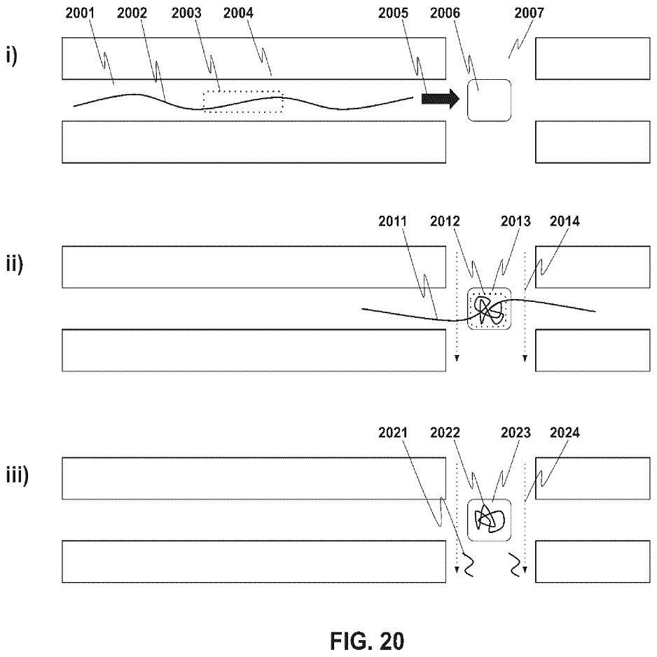

In one embodiment, shown in FIG. 20, a long nucleic acid parent molecule (2002) within an elongation channel (2001) of a confining fluidic device (2004) has an ROI identified (2003). To segregate the ROI, the molecule is transported (2005) by an external force towards an entropic trap (2006) that is also in fluidic connection with a cross-channel (2007). In the preferred embodiment, the long nucleic acid molecule is transported at sufficiently fast velocity over the trap, such that molecule has insufficient time to relax into the trap. However upon registering the ROI over the trap, the external force is removed, and the ROI is allowed to relax into the trap (2013), forming deformable object of coiled nucleic acid (2012). To complete the segregation of the ROI, the non-ROI portions of the molecule can be disconnected from the ROI by targeted photo-cleaving (2021), and then removed via the application of a fluid flow (2024). Alternatively, in some embodiments, a digestive enzyme could be flowed to remove the non-ROI portions of the long nucleic acid molecule. After the non-ROI material has been removed, the ROI(s) can be collected by applying a sufficiently large external force that the ROI(s) can escape the Entropic trap.

In other embodiments, there may be multiple ROIs along the length of the long nucleic acid molecule, and the ROIs are respectively, and simultaneously placed over entropic traps. In order to accommodate different physical distances between ROIs along the long nucleic acid molecule, the device can be manufactured with an array of differently sized entropic traps with different separation distances between the traps. Alternatively, the different sized ROI(s) may be accommodated by trapping individual ROI(s) into multiple traps, such that each trap contains a portion of a ROI(s). For example, along a long nucleic acid molecule, one ROI may occupy 3 traps, and a different ROI may occupy 4 traps.

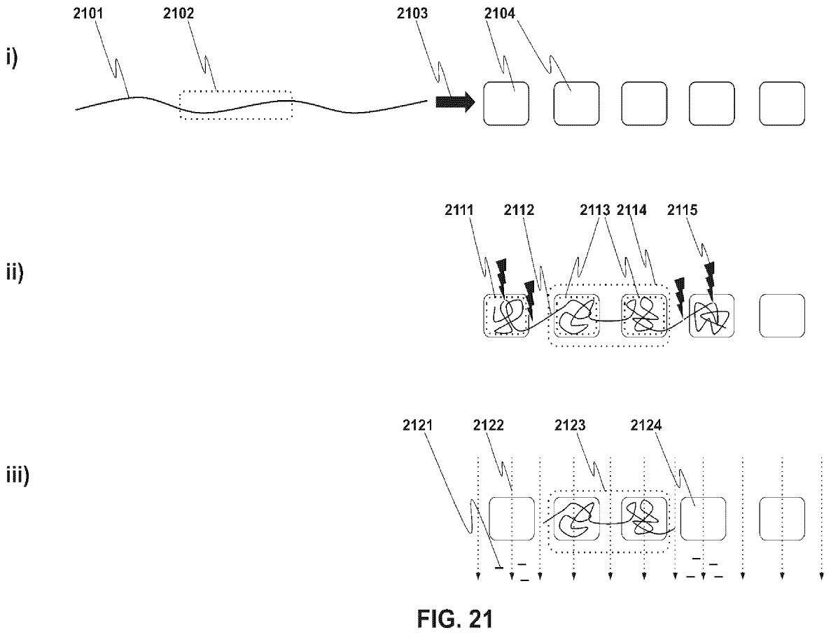

FIG. 21 demonstrates an embodiment where-by a single ROI is captured in multiple traps, as the ROI is too large to be accommodated by a single trap. Here, a long nucleic acid molecule 2101 with an ROI 2102 is transported via an external force 2103 to an array of entropic traps 2104 within a confined fluidic device. The molecule is transported over the array, and then allowed to relax into the array of traps. The ROI 2114 is comprised of two deformable balls of coiled nucleic acid (2113) each in a separate trap. However, in this embodiment where-by there is a surplus of entropic traps in the entropic trap array, non-ROI regions of the molecule also form deformable balls of coiled nucleic acid in trap in a similar fashion 2111. Segmentation of the ROI is then performed by photo-cleaving 2115 the non-ROI material, generating small child molecules 2121 which can escape the trap in the presence of an external force 2122, which is simultaneously not sufficiently strong to escape the larger ROI 2123 from its respective traps.

In another embodiment, the reverse process can be performed where-by the non-ROI regions of the long nucleic acid molecule are trapped, and the ROI(s) separated.

### Capturing Long Nucleic Acid Molecules in Confined Fluidic Devices by Targeted Gel Melting

In this set of embodiment devices and methods, ROI(s) within long nucleic acid parent molecule are segmented and separated from the parent by selectively melting a solidified gel containing the ROI(s) within a confined fluidic device to release said ROI(s). Here, long nucleic acid molecules are flowed into elongation channels in a solution containing a gel that exhibits thermal hysteresis in its liquid-to-gel transition. The long nucleic acid molecules in at least a partially elongated state within said elongation channel are then fully, or partially immobilized in the elongation channel by lowering the temperature. In this state, ROI(s) that have been identified for segmentation and capture can be released from the gel via localized melting of the gel with a focused IR laser around the ROI. In some embodiments, the interrogation of the long nucleic acid to identify the ROI(s) is done while the long nucleic acid molecule is at least partially contained within the gelled material.

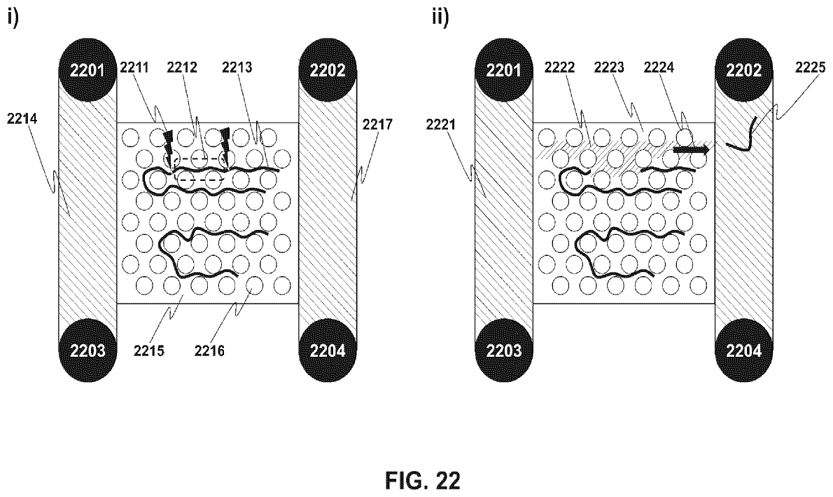

FIG. 22 demonstrates such an embodiment. Here a solution containing the sample of at least one long nucleic acid molecule 2213 and agarose is flowed into an elongation channel (2215) that at least partially elongates the molecules. In this particular embodiment, the elongation channel is in fluidic connection with an inlet channel 2214 and an outlet channel 2217. In this particular embodiment, the elongation channel includes physical obstacles 2216 to promote elongation, although some embodiments may not have such physical obstacles. After loading the elongation channels with the long nucleic acid molecules surrounded by a solution containing agarose, the inlet and outlet channels are purged of the gel-containing solution, by displacing it with a non-gel-containing solution via the fluid connection ports (2201, 2203, 2202, 2204). Next the device temperature is lowered below the gel transition temperature such that the gel solution in the elongation channels solidifies (or semi-solidifies). With the long nucleic acid molecules in at least a partially elongated state such that the ROI(s)(2212) can be identified, the ROI(s) are segmented via the targeted application of photo cleaving 2211 at the ROI boundaries. A focused IR laser is used to melt the region around the ROI, along with a fluidic pathway 2222 from inlet to outlet channels, such that with the application of an external force 2224, the segmented ROI 2225 is able to escape into the outlet channel (or inlet channel), while the remainder of the parent molecule remains immobilized, or has substantially reduced mobility, in the solidified gel.

For all embodiments, the reverse selection can be performed, such that gel is melted for the non-ROI regions of long nucleic acid molecule to first flush out the non-ROI nucleic acid, then afterwards collect the ROI(s). This may be more advantageous if the ROI(s) portions constitute a portion greater than 50% of the overall parent molecule.

For all embodiments the physical state of the environment around the long nucleic acid molecule post-gelling and post-melting need not be completely solid or completely liquid respectively. The requirement is only that a long nucleic acid molecule within the elongation channel of the confining fluidic device exhibit an increase in mobility to an external force in the transition from the ‘gelled’ state to the ‘melted’ state.

### Capturing Long Nucleic Acid Molecules in Confined Fluidic Devices by Un-Caging

In this set of embodiments, at least one ROI within a long nucleic acid molecule confined within an elongation channel of a confining fluidic device is targeted with photons such that caged-affinity groups directly or indirectly bound to the molecule within the ROI become uncaged, and the long nucleic acid molecule is cleaved at the boundaries that define the ROI, thus segregating the ROI. The segregated ROI with at least one un-caged affinity group is then free to bind to a binding partner, and hence capture the ROI.

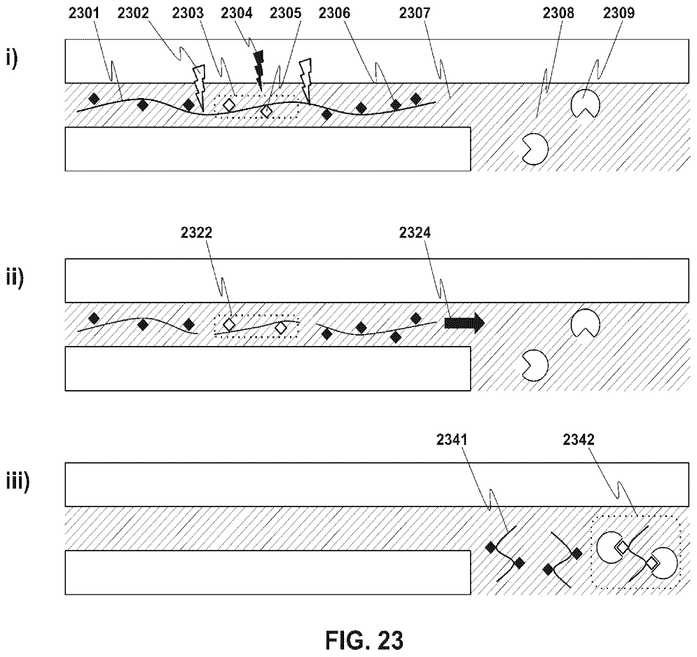

FIG. 23 demonstrates one possible embodiment of un-caging affinity groups on an ROI to capture said ROI. Here, a long nucleic acid molecule 2301 contained within an elongation channel (2307) of a confined fluidic device, has a plurality of bodies bound to it (2306) which contain a photo-liable protecting group. Within an elongated portion of said molecule, a ROI is identified (2303), and within the ROI region the appropriate wavelength (2304) is used to un-cage the affinity group (2305). With the affinity group of the bound bodies within the ROI(s) now un-caged, the ROI(s) can now be bound to other bodies that contain the affinity group's binding partner (2309). In the preferred embodiment, the un-caged affinity group is a biotin, and the binding partner contains streptavidin attached to a magnetic bead. In this particular embodiment, the ROI is segregated from the parent molecule via targeted photo-cleaving 2302 at the ROI boundaries. However, all previous methods of targeted ROI segregation within a confined fluidic device could also be used.

In the embodiment demonstrated in FIG. 23, once the ROI(s) are both segregated and have their caged affinity groups un-caged 2322, the affinity groups can then be bound to their respective affinity partners 2309 in a separate collection fluidic chamber 2308 from the elongation channel. Here, the ROI(s) 2322 are flowed 2324 to the collection chamber, where the binding of the ROIs 2342 to the affinity partners takes place. The method of separation of the ROI(s) 2342 from the non-ROI long nucleic acid molecule(s) 2341 depends on the nature of the affinity partner. In some embodiments, as shown in FIG. 23 the affinity partners 2309 are free bodies in solution, which are then themselves attached to a bead, preferably a magnetic bead, which then allows for separation via a magnetic field. In some embodiments, the affinity partners are attached to a substrate, allowing for separation via rinsing away of the non-ROI molecules.

In some embodiments, the binding to the affinity partners occurs in the elongation channel, in some embodiments binding occurs prior to segregation of the ROIs. In some embodiments, the binding occurs after extraction from the device.

### Parent Molecule Immobilized on an Open Fluidic Device

In the following set of embodiments, the long nucleic acid molecules are combed on an open fluidic device and interrogated to generate a physical map, identify ROI(s), and then target said ROI(s) for segregation and capture. Here, at least a portion of a long nucleic acid molecule is presented in an elongated state on the surface of an open fluidic device by combing said long nucleic acid molecule onto the open fluidic device, allowing for interrogating the molecule's physical map within the elongated portion of the molecule, identifying ROI(s), and then targeting said ROI(s) for segregation from the parent molecule and capture.

In this set of embodiments, the ROI(s) to be targeted are on the surface of the open fluidic device, or are contained within a thin porous film on the surface of the device, or a combination there-of, and thus the ROI(s) are accessible to direct interaction with an applied solution, a photon, or contact probing. In the preferred set of embodiments, the process of interrogating the long nucleic acid molecule's physical map generates a coordinate map of the surface of the open fluidic device in which the long nucleic acid molecules, their physical map, and their respective ROI(s) are located within said coordinate map. Employing such a map, the targeting of focused photons, dispensed solution, or a contact probe can be guided to the desired molecule or ROI on the surface. In the preferred embodiment, the open fluidic device physically engages with a control instrument that interrogates the long nucleic acid molecule's physical map, is the same instrument that directs the targeting of focused photons, dispensed solution, or contacting probing, such that all electrical mechanical systems within the instrument can share the same coordinate space to target molecules and ROIs within the coordinate map. In some embodiments, the targeting is performed on a different instrument from the interrogation instrument, and fiducials on/within the open fluidic device are used to register the coordinate map.

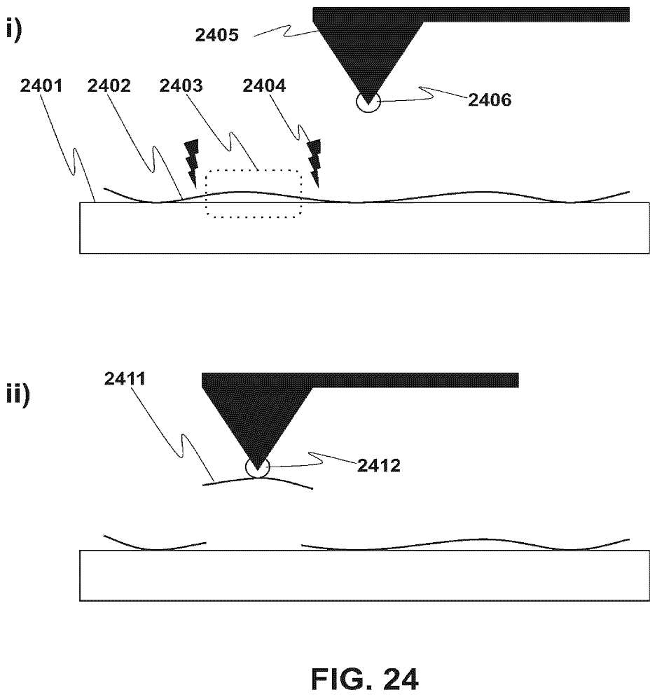

FIG. 24 demonstrates an embodiment where-by a combed long nucleic acid molecule 2402 on the surface of an open fluidic device 2401 has ROI 2403 identified for capture. In this embodiment, the ROI is segregated via photo-cleaving 2404 of the boundaries of the ROI Once segregated, the contact probe 2405 with a functionalized point 2406 is lowered and positioned to contact the ROI using the previously registered ROI coordinates on the surface of the fluidic device. The contact probe contacts the ROI molecule 2411 under conditions that allow the molecule to bind to the functionalized point 2412, such that the contract probe can retract from the surface with the ROI.

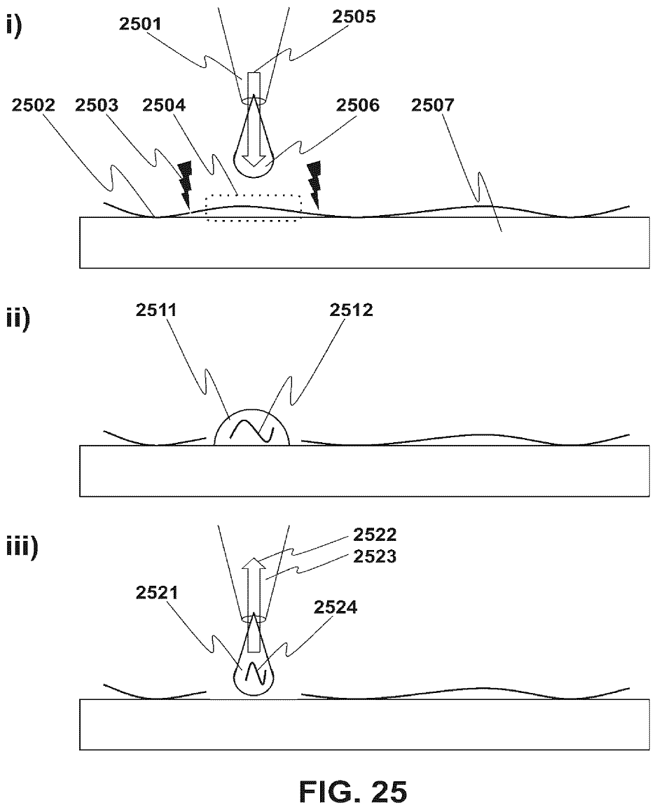

FIG. 25 demonstrates an embodiment where-by a combed long nucleic acid molecule 2502 on the surface of an open fluidic device 2507 has ROI 2504 identified for capture. In this embodiment, the ROI is segregated via photo-cleaving 2503 the boundaries of the ROI and submerged in a dispensed solution 2506 dispensed 2505 from a dispenser 2501. Once submerged and segregated, the ROI 2512 is re-suspended in the solution drop 2511 on the surface of the open fluidic device. The drop 2521 containing the ROI 2524 can then be extracted 2522 from the surface with an extractor 2523. In some embodiments, an oil is then dispensed on the fluidic device, covering the drop, which maintains contact with the device surface, and the extractor extracts the drop by pushing through the oil.

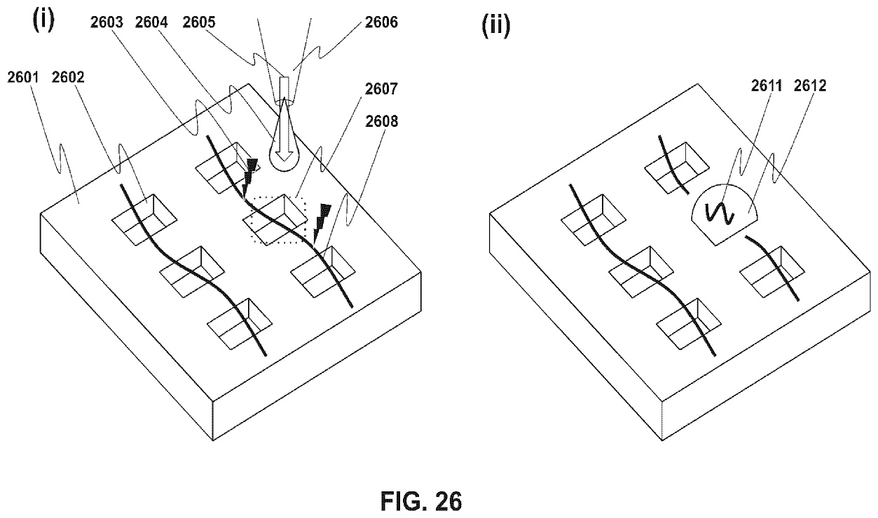

In some embodiments, the long nucleic acid molecules are combed on an open microfluidic device that includes patterning topological and/or surface energy modifications to form wells on the surface of the device, so as to physically contain the dispensed solution within said wells. FIG. 26 demonstrates an open fluidic device 2601 with fluid capture wells 2602 onto which long nucleic acid molecules are combed 2608 and then interrogated to identify ROI(s) 2607. In this embodiment, the ROI is segregated via photo-cleaving 2603 the boundaries of the ROI and submerged in a dispensed solution 2604 dispensed 2605 from a dispenser 2606. Once submerged and segregated, the ROI 2611 is re-suspended in the solution drop 2612 within the well of the fluidic device.

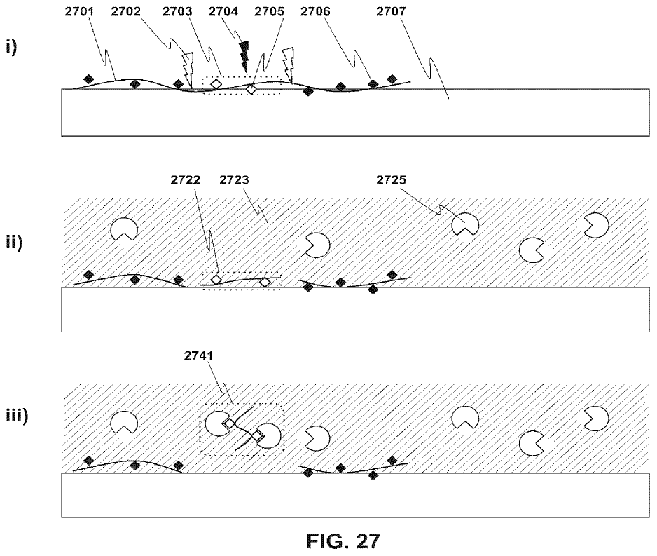

In another embodiment, FIG. 27 demonstrates a long nucleic acid molecule 2701 combed on the surface of an open fluidic device 2707, where said molecule is bound to by a plurality of bodies 2706, each of which is attached to a caged affinity group protected by a photo-liable protecting group. In this embodiment, the ROI 2722 is segregated via photo-cleaving 2702 the boundaries of the ROI and the caged affinity groups along the ROI are un-caged 2705 via the targeted exposure of photons 2704.

Once the ROI 2722 is both segregated and at least one of its affinity groups uncaged, the ROI can then be captured by exposing the ROI to a solution 2723 containing affinity-partners 2725, such that the un-caged affinity groups on the ROI bind to the affinity-partners to form a group 2741. In one embodiment, the affinity-partner includes a magnetic bead such that the group can be collected with a magnetic field. In some embodiments, after the ROI has had its affinity groups un-caged and become segregated from the parent, the ROI is first rinsed off the surface of the open fluidic device, and then collected by binding to affinity partners. In some embodiments, the affinity partners are attached to a substrate.

### Long Nucleic Acid Molecule Continuity Via Tracking of Segmented Child Molecules

In many applications it is advantageous to segment a long nucleic acid parent molecule into smaller children molecules, while maintaining knowledge their originating position within the parent molecule, their positional order with respect to each other in the parent molecule, their relative distance in base-pairs between each other in the parent molecule, or combination there-of. The following embodiments describe various methods and devices for achieving some or all of these goals. In some embodiments, tracking knowledge from all children from the parent is maintained. In some embodiments, tracking knowledge from only a subset of children from the parent is maintained.

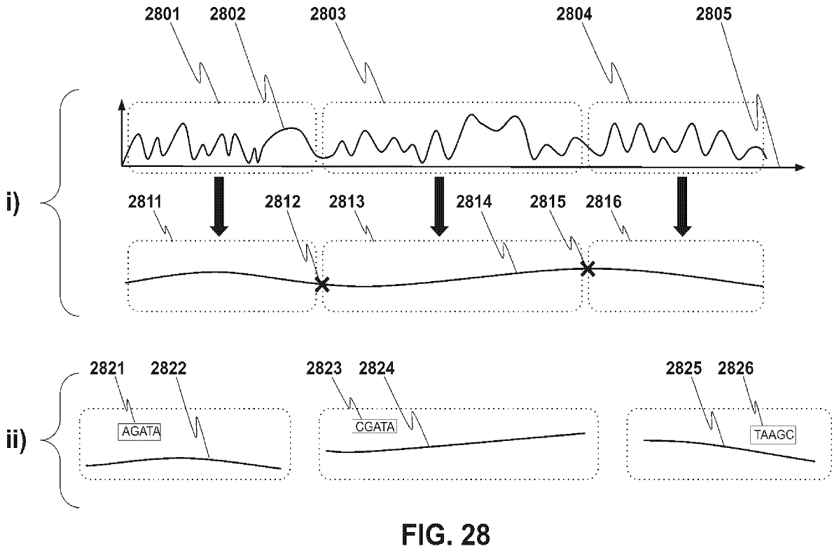

FIG. 28 demonstrates one embodiment method of tracking child molecules from a parent molecule. Here, a long nucleic acid parent molecule 2814 has been interrogated to generate a physical map 2802, in which the physical map represents information that correlates with the underlying genomic information of the parent molecule, along the physical length 2805 of the parent molecule. The molecule is then cleaved at points (2812, 2815) to generate three children (2811, 2813, 2816). The cleaving points can be selected at random, or by some controlled process. In some embodiments, the controlled process is at least partially informed by an analysis of the physical map. In some embodiments, the sizes of the children are selected to enable a downstream enzymatic process. In the preferred embodiment, knowledge of the cleaving sites within the physical map is known, such that individual physical maps of the generated children are then also known (2801, 2803, 2804). However, in some embodiments, the children may be interrogated to generate their respective physical map after the are created from the parent.

Once the children are created from the parent, in some embodiments each child is assigned a unique barcode to be associated with each child. For example, in FIG. 28, barcode 2821 is associated with 2822, barcode 2823 is associated with 2824, barcode 2826 is associated with 2825. In some embodiments, the association is one of physical proximity, for example the barcode shares an isolating region with the child (eg: a droplet, a an entropic well). In some embodiments, the association is a bond between the barcode and the child. In some embodiments, a sub-set of children receive the same barcode. In some embodiments, the unique barcode comprises a unique combination of unique barcodes. In the preferred embodiment, the content of a barcode that is assigned to a particular child is known, such that there exists a means of generating a lookup table of barcodes to child relations.

### Segmenting and Capture of Child Molecules Via Entropic Traps in a Confined Fluidic Device

In this set of embodiment devices and methods, an originating (parent) long nucleic acid molecule is physically segmented with entropic traps. A long nucleic acid molecule in the presence of an array of entropic traps, with no substantial externally applied force, will naturally occupy the traps as that is the lowest energy state of the molecule. The amount of nucleic acid that occupies each trap depends on each trap's respective size, the molecule's physical properties, and composition and temperature of the surrounding solution [Reisner, 2009]. As such by flowing long nucleic acid molecules over such traps, and then removing the external force, the molecules will relax and self-assemble into the traps. A highly beneficial aspect of this embodiment is that the quantity of nucleic acid in each trap will have a maximum, limited by the trap size, allowing for simple partitioning of the parent molecule.

Furthermore, the device can employ regions of different trap sizes allowing for the originating nucleic acid molecule to be guided to the desired region, and thus desired segment size, or segment size distribution.

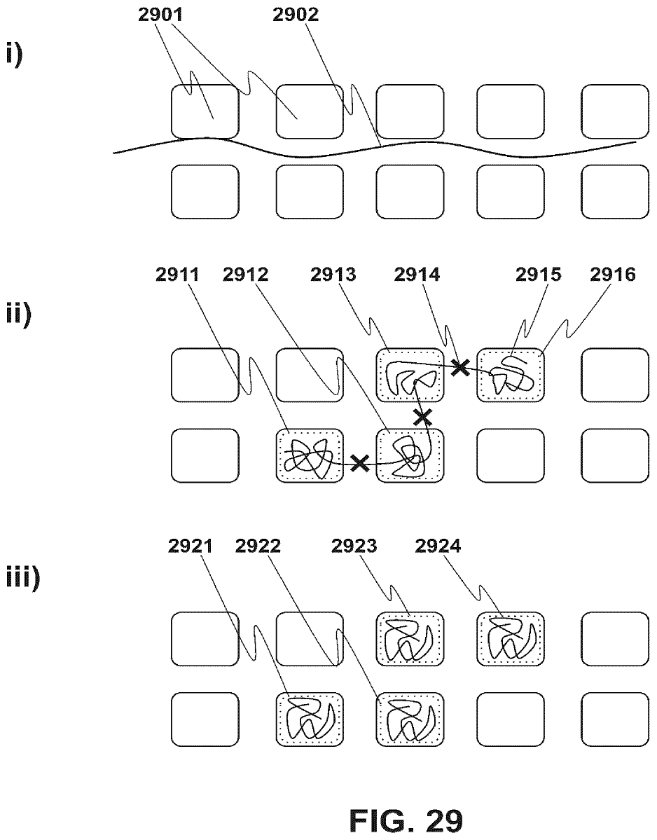

An example embodiment is shown in FIG. 29. Here, a long nucleic acid molecule 2902 in at least a partially elongated state is transported over an array of entropic traps 2901. The sizes of the traps are designed to accommodate a desired quantity of nucleic acid. Once the molecule is over the array, and the external force removed or diminished, the molecule will relax, and occupy the traps, forming deformable balls of coiled nucleic acid in each. By interrogation, the physical relationship of the trapped sections of with respect to their order in the parent molecule 2916 can be determined and recorded. Once the segments have formed, the interconnecting sections of nucleic acid between the traps can be cleaved, here in this embodiment by photo-cleaving (2914), resulting 4 child segments: 2911, 2912, 2913, 2916. In some embodiments, the molecule 2915 can have its physical map interrogated prior to photo-cleaving, such that the elongated portions of the molecule that connect the deformable coils in the traps will have a physical map signature, which can then be used to identify the boundaries of the children within a map generated from a previous interrogation, and then to determine the children's respective maps.

The array of entropic traps can be ID or 2D, and need not be regularly spaced in either direction. They can be identical in size, or differ. They can take on any shape, such as, but not limited to: box, cube, rectangle, cylinder, cone. They do not need to be symmetrical in shape. Their dimensions along any axis can be as small as 10 nm, and as long as 50 microns. Their volume can vary from 1 atto-liters to 1 nano-liter. The separation distance between adjacent traps can range from 50 nm to 500 microns. The amount of nucleic acid that falls into each trap is determined by a number of factors including the DNA persistence length (which is a weak function of buffer conditions) the dimensions of the trap, the spacing between adjacent traps and the degree of entropic restriction imposed on the portions of the nucleic acid that bridge the inter-trap regions [Reisner, 2009]. For example the same size trap will hold less DNA if it is within a few (<10) microns of an adjacent trap, if the volume of the trap is smaller, or if the regions between traps is a 2D nanoslit then a larger nanoslit height will also result in less DNA occupying the same trap. While there is no upper limit on trap size, 1kb represents an approximate lower limit of the amount of DNA that can be segmented into each pit of an array of reasonably spaced pits.

Once inside the entropic traps, specific forces and/or reagents may be targeted to specific segments of long nucleic acid molecule and/or portions of the molecule. For example, by directing a laminar flow of reagents to flow over a particular entropic trap, or a particular region between traps, and thus exclusively exposing the desired section of long nucleic acid molecule to the reagents. Such an embodiment is advantageous, in that once the molecule has occupied at least one trap, there exists a flow rate for the delivery and exchange of reagents such that the molecule will not escape from the trap.

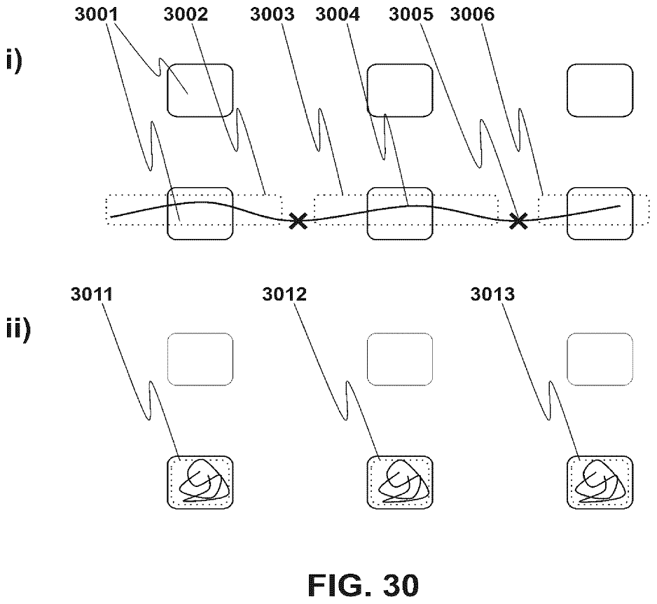

In some embodiments of segmentation as shown in FIG. 30, the long nucleic acid molecule 3004 can be transported over an array of traps 3001, and then photo-cleaved prior to relaxation into the traps. With such an embodiment, the desired boundaries between the child segments (3002, 3003, 3006) where the photo-cleaving is to occur (3005) can be chosen with greater flexibility. In addition, the physical map of each child can be captured prior to photo-cleaving and subsequent children (3011, 3012, 3013) relaxing into deformable objects of coiled nucleic acid in their respective traps.

Once the children are segmented into individual entropic traps, they can be released simultaneously via an application of sufficiently large external force to escape them. Tracking of the individual children can be accomplished via their individual physical map and/or binding of unique, and known barcodes to each child.

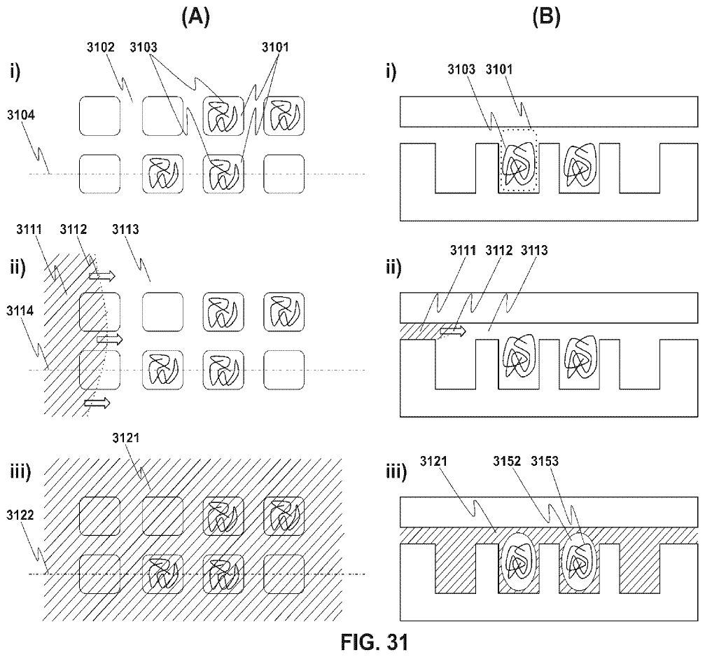

FIG. 31 demonstrates an embodiment device and method for forming droplets from the child molecule segments 3103 in entropic traps 3101 by displacing the surrounding aqueous liquid environment with an oil. (FIG. 31(B)(i) is as cross-section of FIG. 31(A)(i) at 3104, FIG. 31(B)(ii) is as cross-section of FIG. 31(A)(ii) at 3114, and FIG. 31(B)(iii) is as cross-section of FIG. 31(A)(iii) at 3122.) Upon entry of an oil solution 3111 into the confining fluidic device, oil will displace3112 the aqueous solution 3113 within the channel, and in the wake of the oil displacement, water-in-oil droplets 3152 will form in the traps [Amselem, 2016], resulting in each child molecule being contained in its own droplet 3153, surrounded by oil 3121.

The droplets are deformable objects in an entropic trap, and as such, they can be released from their respective trap with a sufficiently large force. In some embodiments, the droplets are released almost simultaneously with a sufficiently large external force applied on all droplets. In some applications, an embodiment to have addressable release the desired droplet is required. In one embodiment, an agarose gel is incorporated in the aqueous solution, and the cooling the confining fluidic device post-droplet formation, gels the contents of the droplets rendering them solid/semi-solid [Amselem, 2016]. With this solid internal state, the droplets have a higher energy requirement to deform them when compared to a liquid internal state. Then by selectively melting gel within a selected droplet, there exists an applied force sufficiently large to escape the melted droplet, but not the solid droplet. In this embodiment, a focused IR laser, in combination with the appropriate level of applied external force, can be used to escape the desired droplet.

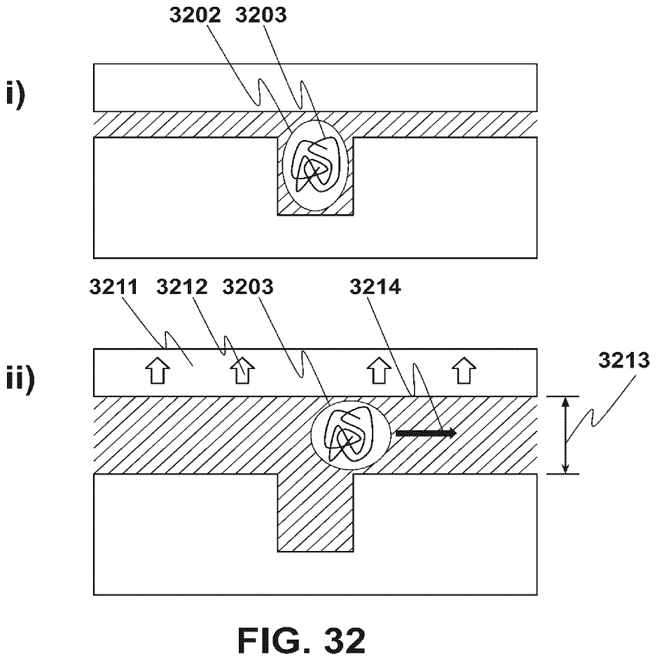

In another possible embodiment demonstrated in FIG. 32, a water-in-oil droplet 3202 containing a child segment 3203 in an entropic trap of a confined fluidic device can be released from the trap by reducing the entropic barrier. In this embodiment, the barrier is reduced by modulating 3212 the position of a channel wall 3211 such that the confining dimension 3213 is increased, and thus lowering the entropic barrier such that the droplet can escape from the trap with an external force 3214 which would have been insufficient to escape the droplet prior to the modulation. There are a number of different devices and methods for modulating the physical position of channel walls (see previous section on use of such confining dimension modulation for “preparing for interrogation”). In the preferred embodiment, the modulation can be limited to regions within the fluidic device, where-by each region is associated with at least one entropic trap, and each region is individually addressable.

### Separation and Capture of Child Molecules Immobilized in an Open Microfluidic Device

In the following set of embodiments, the long nucleic acid molecule in an open fluidic device is segregated into children molecules in a manner such that information associating a child's originating position within the parent molecule and/or relative order with the other children is maintained with the child as the child is separated and removed from the surface (captured). In the preferred embodiment, the parent long nucleic acid molecule is interrogated to generate a physical map, either before or after the segmentation of the parent into children, but with knowledge of the segmentation boundaries in the map maintained such that the individual child's physical map, and the map's orientation with respect to the other children, is known.

In this set of embodiments, the children to be segmented from the parent are on the surface of the open fluidic device, or are contained within a thin porous film on the surface of the device, or a combination there-of, and thus the children and their boundaries are accessible to direct interaction with an applied solution, an focused photon, or contact probing.

In the preferred set of embodiments, the process of interrogating the long nucleic acid molecule's physical map (and/or the respective children's map) generates a coordinate map of the surface of the device in which the children and their respective boundaries are located within said coordinate map. Employing such a map, the targeting of focused photons, dispensed solution, or contact probe can be guided to the child on the surface.

In the preferred embodiment the children are segmented from the parent long nucleic acid molecule by photo-cleaving or exposure to restriction enzymes. Once the children are segmented, they can then be removed (captured) from the surface of the open fluidic device. In some embodiments, all children are individually captured. In some embodiments, only a sub-set of children are individually captured.

In one embodiment, a child is captured using a contact probe, as previously described for Figure $AH, where-by the ROI is child. In one embodiment, a child is captured using absorption into a drop of solution, and then solution capture, as previously described for FIG. 25, where-by the ROI is a child. In one embodiment, a child is captured using absorption into a of solution contained within a patterned well, as previously described for FIG. 26, where-by the ROI is a child,

### Long Nucleic Acid Molecule Continuity Via Barcoding

In this set of embodiment devices and methods, regional unique barcodes (RUBs) are used to tag regions of a long nucleic acid molecule such the relative physical relationship between the regions along the molecule can be obtained via down-stream sequencing. MDA is known to cause issues with downstream bioinformatic analysis due to its non-linear amplification [Huang, 2015]. This makes assembling complicated genomes with large numbers of copy numbers especially challenging. By incorporating RUBs into the primers used for amplification (eg: MDA primers), significant ambiguities in the sequence data can be reduced or eliminated.

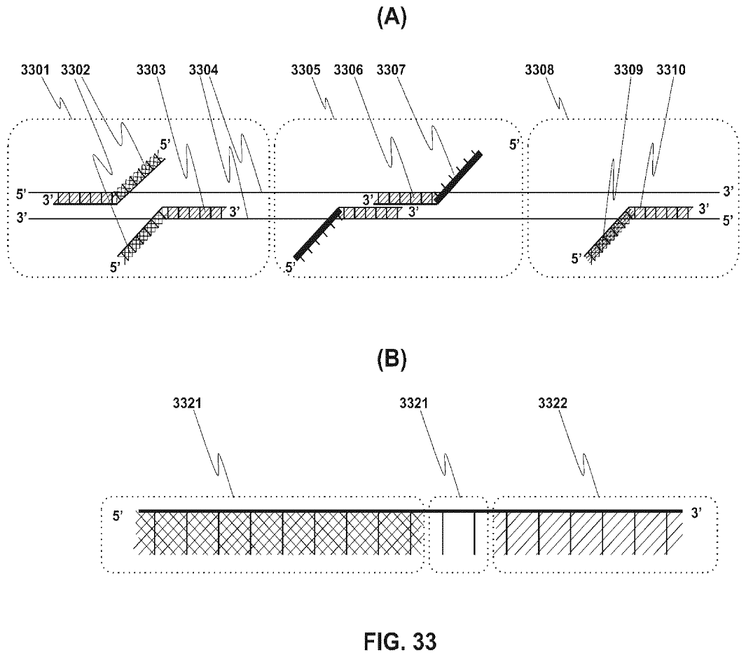

FIG. 33 shows an embodiment where-by long nucleic acid molecule 3304 (shown here with both strands) is divided into 3 regions 3301, 3305, and 3308. Within each region, at random positions, are bound universal primers (3303, 3306, 3310) comprised of which is a barcode that is unique for each region (3302, 3307, 3309) within the molecule. In one embodiment shown in FIG. 33(B), the primer of a universal primer segment (3322), an optional connection segment (3321), and then a barcode segment (3321). In some embodiments, the universal primer contains a 6-base (hexamer) sequence. In some embodiments, the last two bases of each barcoded primer contain thiophosphate modifications, which protect the primer from the 3′ exonuclease activity of Phi-29 nucleic acid polymerase. In the preferred embodiment, the barcode sequence is 4-24 bases in length.

In some embodiments, the barcode contains a PCR sequence target that can then be used for targeted amplification with PCR primers after MDA using said universal primers. In the preferred embodiment, the PCR sequence target within the barcode is identical for all combinations of barcodes.

It has been shown in prior art [Dean, 2002] that ds-nucleic acid when exposed to MDA (universal) primers in a sufficiently high concentration of alkaline solution, that the ds-nucleic acid molecule sufficiently denatures so to allow the MDA universal primers to hybridize to the exposed ss-nucleic acid strand. Further, it has also been shown in prior art that this process can still be employed when the universal primers have additional sequencing information (barcodes, UMIs, eg) [Chen, 2011, U.S. Pat. No. 9,469,874]. Thus, upon sequencing the stands in this embodiment, their regional origin can be determined within the larger nucleic acid molecule by the barcode content. In this embodiment, the transition of RUBs need not be distinct from one region to the next. Depending on the process used for applying the primers, there may overlap of RUBs between adjacent regions. However, this can be accounted for in downstream bioinformatics of the sequencing data.

In some embodiments, the reagent solution in which the universal primer binds to the long nucleic acid molecule, or when the primer extension occurs with a polymerase, includes a recombinase enzyme to form D-loop as described by [Chen, 2016] such that a localized, stable de-natured portion can be maintained.

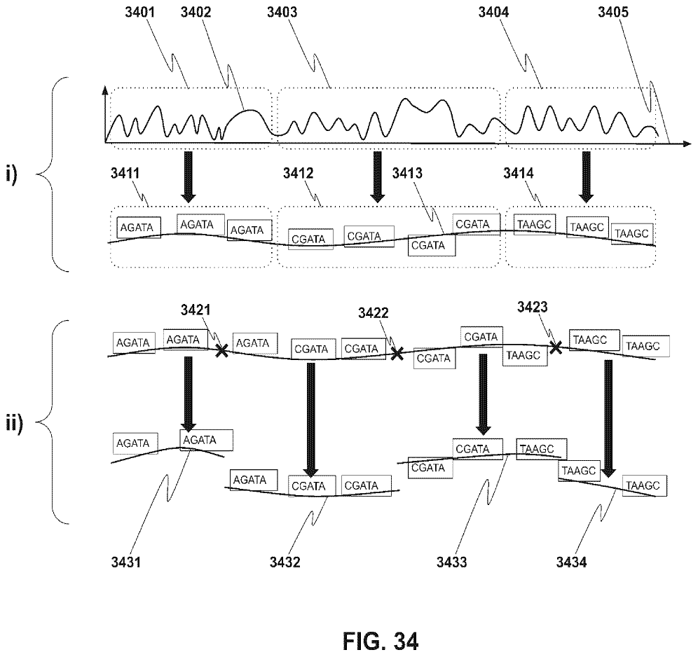

FIG. 34 demonstrates one embodiment, where-by a long nucleic molecule 3413 is divided into 3 regions 3411, 3412, and 3414, each of which is assigned its own unique barcode universal primer. In the preferred embodiment, the long nucleic acid molecule has also been interrogated such that a physical map 3402 has been generated, in which there is informational content that correlates with the molecule's underlying genetic information, along the length of the molecule 3405. In some embodiments, the choice of regional boundaries is at least partially determined via an analysis of the physical map. For example, the physical size of the region may be adjusted depending on the degree of complexity the content may present for sequencing assembly. In some embodiments, there is no physical map of the long nucleic acid molecule generated prior to defining the regions, or if such a map has been generated, it's not used in the determination of the regions.

Once the regions have been defined and each region appropriately barcoded, in some embodiments, the long nucleic acid molecule is then segmented by cleaving (3421, 3422, 3423) into child molecules (3431, 3432, 3433, 3434). In some embodiments the cleaving sites may be chosen at least partially due an analysis of the physical map, or at least partially due to the regional boundaries. In some embodiments, the cleaving sites are randomly selected by a process that generates a known distribution of different sizes. The actual number of unique barcodes can be increased or decreased based on the type of application and requirements for uniqueness within the genome being sequenced. The size of the regions can be tuned for the needs of the application, and the sequencing requirements downstream depending on the complexity of genome, for example the amount of copies or repeats. A region can vary from 10 bp to 1000 Mbp where the ‘region’ may be an entire chromosome, or all chromosomes from a single cell. This can be highly advantageous for applications where-by the nucleic acid material is translocated or copied through genomic rearrangements from one chromosome to another chromosome, as the barcoding of the original genomic content from the cell will allow the down-stream sequencing application to determine the chromosome origin of the long nucleic acid molecule without bias from a reference. In some embodiments the region size is consistent for a particular sample. In some embodiments, the region size can be selected by the user. In some embodiments the region size can be random, or can change do to some criteria. The number of RUB(s) bonded to a region can vary from region to region. In some embodiments, as few as one RUB may be associated with a region. In some embodiments two or more, or 10 or more, or 100 or more, or 1,000 or more, or 10,000 or more, or 100,000 or more.

Furthermore, depending on the down-stream needs of the final application, RUBs may be re-used. For example if there are 4 unique RUBs: {A, B, C, D} which each identify a region approximately 10 kbp in length, which are then cycled with a known pattern (eg: A=>B=>C=>D=>A . . . } this information can guide any downstream sequencing assembly, as any ‘assembly solution’ determined by the bioinformatics assembly that does not demonstrate such a cycling of barcodes in that order approximately every 10 kbp will be known to be erroneous. This embodiment thus provides an effective means of validating sequence assemblies.

In some embodiments not knowing the identity and order in which the RUBs are incorporated, only that the RUBs will regularly change at fixed length scales along the length of the long nucleic acid molecule, provides valuable information for downstream bioinformatics, again as any bioinformatics assembly solution that does not demonstrate this random cycling of RUBs at regular lengths scales will be erroneous.

In some embodiments the RUB can be an inserted piece of nucleic acid into the long nucleic acid molecule, for example via process that uses a transposon or Crispr system to insert RUIs.

### Molecule Confined Within a Confined Fluidic Device

The following set of embodiments describes various methods and devices for binding RUB(s) to regions along the length of a long nucleic acid molecule in a confined fluidic device.

### Bonding of Regional Unique Barcodes Via Targeted Exposure of Reagents

Here we disclose embodiment devices and methods for hybridizing universal primers with barcodes within specific regions of a long nucleic acid molecule via the targeted exposure of reagent solution flow within a confined fluidic device, where-by the reagent solution can include at least one RUB universal primer. In some embodiments, the reagent solution is also comprised of components to promote the de-naturation of ds-nucleic acid.

In one embodiment device and method shown in FIG. 8, a long nucleic acid molecule fragment 813 has a tail section exposed to a reagent solution (816) in a reagent channel (814) flowing from (802) to (803). In this embodiment, the reagent solution contains RUB universal primers in an alkaline solution, that can vary in concentration and barcode composition with time and on demand. Here, an external force on the long nucleic acid molecule is the fluid flow (815) of the reagent solution flow on the tail of the molecule. The retarding force (812) on the molecule maintains at least a portion of the molecule in a delivery channel 811 while the external force pulls the molecule taut. Should a portion of the tail break from the parent long nucleic acid molecule, forming a child molecule, either by design, or at random from stress, the remaining tail can be elongated via the application of an electro-kinetic force applied between (801) and (803). Furthermore, such an electro-kinetic force between could be used to retrack the tail from the reagent exposure if desired.

In other possible embodiments, the retarding force is an entropic barrier (822) interacting with a long nucleic acid molecule (823), or a collection of physical obstacles (832) interacting with a long nucleic acid molecule (833). In the preferred embodiment, the flow of barcode reagents 815 both exposes the tail section of the long nucleic acid molecule in the reagent channel to the barcodes for hybridization, and simultaneously pulls out additional nucleic acid molecule length from the delivery channel 811. The flow rate, barcode concentration, and exposure time can all be tuned as desired to achieve the desired barcode binding coverage along the tail. Once a sufficiently long section of the nucleic acid tail has been exposed to the specific RUB universal primers, the tail section can then be released from the parent molecule via photo-cleaving to produce a child molecule uniquely bound to by the selected RUB. Once released, additional tail material can then be introduced into the reagent channel, for example via application of an external force (for example, an electric field from 801 to 803), and the reagent solution flow composition can be changed to a different RUB.

By continuing this process along the length of the long nucleic acid molecule, and keeping track of which RUBs was used, child segments of nucleic acid molecules hybridized to known RUBs are generated and can be collected for amplification and/or sequencing.

In another embodiment device and method, demonstrated by FIG. 7(A), a long nucleic acid molecule 702, in an elongation channel 701 is transported through an intersection with a cross-flow reagent delivery channel 705 in which portion of the molecule exposed to the reagents is in a substantially elongated state 708, and in which the reagents comprise RUB universal primers 704 of varying concentration and composition. Such an embodiment may be operated in any number of ways.

In one embodiment, different regions of the long nucleic acid molecule can be defined by different RUBs by controlling the translocation speed of the long nucleic acid molecule through the elongation channel via an external force 706, while coordinating a change in the RUB composition in the reagent flow. Various combinations of coordinated molecule movement and reagent flow rate and composition are possible. In some embodiments, the molecule movement through the intersection is with a constant velocity. In some embodiments, a stepping movement is used. In another embodiment, a long nucleic acid molecule may be exposed to multiple reagent delivery channels simultaneously, with each channel comprising a different RUB.

### Bonding of Regional Unique Barcodes Via Proximity to an Array of Barcode Pads

In another set of embodiment devices and methods, at least a partially elongated portion of a long nucleic acid molecule is brought into proximity with an array of pads within a confined fluidic device, where-by each pad is associated with a specific RUB universal primer, connected to the pad via a cleavable linker. In the preferred embodiment, the linkers are photo-cleavable. In the preferred embodiment, the specific RUB associated with each pad is known. In some embodiments, the linkers are cleaved after hybridization to the long nucleic acid molecule. In some embodiments, the linkers are cleaved before hybridization.

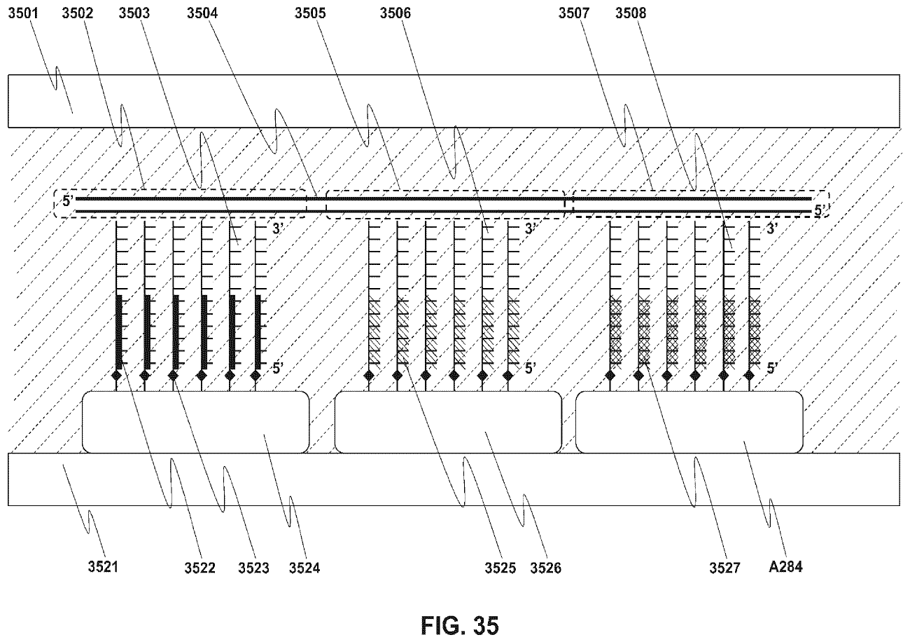

FIG. 35 demonstrates an embodiment where-by a long nucleic acid molecule 3504 is brought into proximity, or contact with, an array of pads (3524, 3526, 3528) contained within an elongation channel of a confined fluidic device. In this particular embodiment each pad within the device is associated with a unique RUB (3522, 3525, 3527), each with a respective universal primer (3503, 3506, 3508), all of which are connected to their respective pad via a photo-cleavable linker 3523. In some embodiments, the long nucleic acid molecule comes into proximity with pads via the confining boundaries of the elongation channel. In the preferred embodiment, the confinement dimension of the channel is less than 50 nm, or less than 25 nm, or less than 10 nm. In some embodiments, an external DEP force may be applied on the molecule to achieve proximity. In some embodiments the elongation channel dimensions may be modulated, as discussed previously in processes for “preparing for elongation”. The embodiments where-by the confining dimension can be modulated are particularly advantageous, as the long nucleic acid molecule can be brought within 10 nm, or within 5 nm, or within 2 nm of the RUB universal primers. In all embodiments, the region sizes (3502, 3505, 3507) are defined by the pad geometrics and physical interaction with the long nucleic acid molecule 3504.

In some embodiments, the pads are comprised of beads. In some embodiments each bead can also include a unique combination of fluorescent colors that correspond to the unique barcode of each RUB such that the particular RUB and its physical location can be identified if desired. In some embodiments, the beads can be flown into a fluidic channel of the confining fluidic device, the channel having a cross-section dimension of sufficiently small in size that the beads must transit through the channel in a single-file fashion. Once the beads are in position, the long nucleic acid molecule can then be transported over the beads in the same channel, and then brought into proximity with the RUB universal primers.

### Molecule Immobilized in an Open Fluidic Device

The following set of embodiments describes various methods and devices for binding RUB(s) to regions along the length of a long nucleic acid molecule in an open fluidic device.

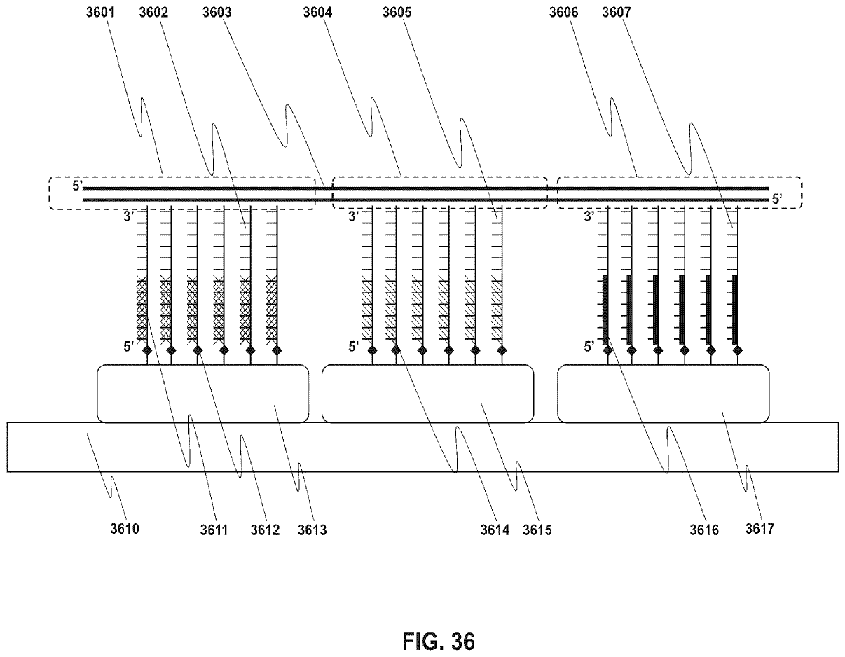

In one embodiment demonstrated in FIG. 36 a least one long nucleic acid molecule 3603 is combed on a surface of a substrate 3610 that is patterned with an array of pads (3613, 3615, 3617), where-by each pad is associated with a unique RUB (3611, 3614, 3616), each with a respective universal primer (3602, 3605, 3607), all of which are connected to their respective pad via a photo-cleavable linker 3612. In this particular embodiment, the size of the pads, along with the alignment of combed long nucleic acid molecule on the pads defines the regions (3601, 3604, 3606), such that each region within the molecule will be hybridized to a specific RUB universal primer. In one particular embodiment, each pad is positioned within a patterned well on the surface of the open fluidic device, with each well defined by surface energy variation and/or topological variation such that a drop of solution can be contained within the well. In this embodiment, after the long nucleic acid molecule is combed on the surface, over the wells of pads, drops of solution are dispensed into each desired well, and the cleavable linker connecting the RUB universal primer is cleaved, allowing the universal primers to be suspended in the solution drop and bind to the long nucleic acid molecule. In this embodiment, regions are defined by the drop. In some embodiments, the combed molecules are in physical contact with the RUB universal primers immediately after combing. In some embodiments, the combed molecules are in proximity to the primers immediately after combing, suspended over the primers that are contained within the well.

In some embodiments, the long nucleic acid molecule is combed on the surface of an open fluidic device, and then RUB universal primers are brought into proximity with the combed molecule. In one embodiment, the RUB primers are attached to pads on a patterned substrate, and said substrate is then brought into contact with the combed molecule, with the alignment of the pads and molecule defining the regions. In one embodiment, the RUBs are brought into contact with RUB primers by dispensing a solution of primers onto the combed molecule, with the drop of solution containing a unique RUB, and the intersection of the molecule and the drop defining the region.

### Droplet Devices and Methods for Long Nucleic Acid Fragments

The following embodiments devices and methods pertain the controlled encapsulation of long nucleic acid molecules into a single droplet. In some embodiments only a single long nucleic acid molecule is encapsulated in a single droplet. In addition, embodiment devices and methods are disclosed that allow for the association of a known unique barcode (or unique signature) with a specific droplet, such that that specific droplet can be uniquely tracked.

### Long DNA Concentration and Encapsulation in a Droplet

Generating a droplet with a single long nucleic acid molecule has been demonstrated previously [Lan, 2017], however in order to reduce the probably of having droplets with multiple molecules encapsulated, this process relied on using a source solution of low concentration of nucleic acid molecules such that the Possion distribution of droplet occupancies were such that the majority of the droplets were vacant. Similarly, injecting a solution containing a low concentration of nucleic acid molecules into a droplet [Weitz, 2009, U.S. Pat. No. 9,757,698], would also rely on Poisson statistics of the injection solution nucleic acid concentration to manage the droplet occupancy distribution. Here we describe various embodiment devices and methods designed to control the concentration of long nucleic acid molecules locally at the point of encapsulation, such that the concentration of long nucleic acid molecules can be independently controlled, separate from the encapsulation mechanism. In addition, long nucleic acid molecules fragments can be fluorescently stained with a dye so they can be imaged and identified at the single molecule level, thus allowing for confirmation of the encapsulation event, enabling a feedback system to modulate the process.

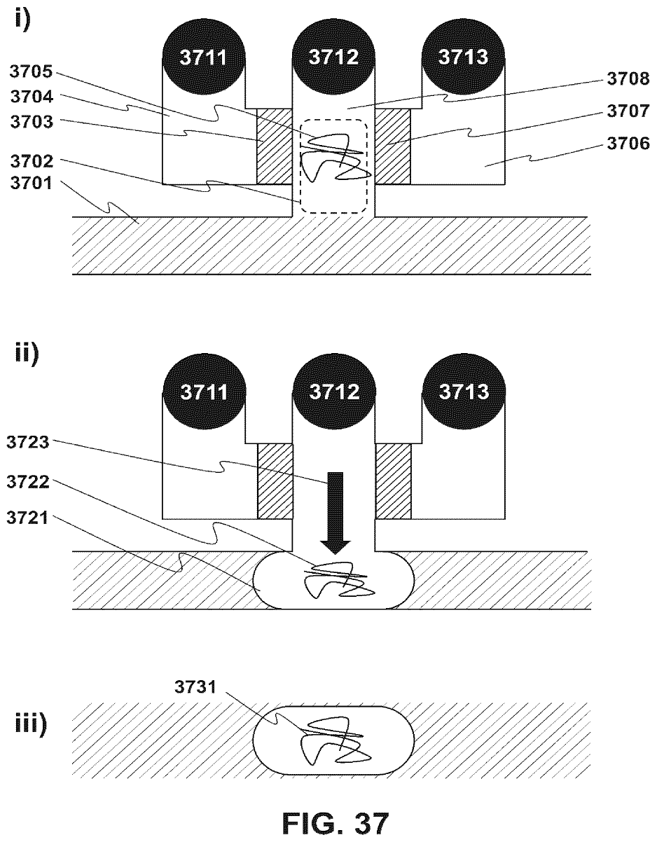

In one embodiment device and method demonstrated in FIG. 37, a cross-channel is used such that a long nucleic acid molecule can be pre-concentrated against an entropic barrier at the point of encapsulation. Once confirmed visually via fluorescent imaging that the long nucleic acid is suitably located at the encapsulation region, the molecule can then be encapsulated at will, with fluorescent imaging employed to confirm nucleic acid molecule encapsulation. In this embodiment, an aqueous solution droplet generating channel 3708 is in fluidic connection with an oil droplet transport channel 3701. The two fluid channels are maintained at pressure equilibrium when droplets are not being formed. To form a droplet, a pressure increase from fluidic connection port 3712 flows the aqueous solution into the oil channel to generate a water-in-oil droplet, where-by the contents of the droplet consists of the contents within the encapsulation site 3702, a region located in the droplet transport channel, immediately adjacent to the droplet transport channel. To enable the formation of droplets with a controlled occupancy of long nucleic acid molecules, a nucleic acid delivery cross-channel 3704 and 3706 are in fluidic connection with droplet generating channel 3702, within close proximity of the encapsulation site 3702. There are multiple ways of operating such a device, depending on the configuration of the embodiment. There are two entropic barriers 3703 and 3707, of which both, or either of which, or none, may be present. If an entropic barrier is not present, then its respective nucleic acid delivery channel is in direct fluidic contact with the droplet generating channel. In the embodiment where-by entropic barrier 3707 is present, and 3703 is not, a long nucleic acid molecule 3705, originating from fluidic port 3711, is transported to the encapsulation site 3702, via an external force applied from 3711 to 3713, such that molecule is brought up to the barrier 3707, but the force is insufficient for the molecule to pass. Either by maintaining the same level of force, or less, or none, the molecule will remain at the encapsulation region until a droplet 3721 is generated that encapsulates the solution and molecule 3722 in the encapsulation site, via an applied pressure 3723. The result is a water-in-oil droplet containing the long nucleci acid molecule 3731. In some embodiments, the geometry of the encapsulation region 3702 allows for a nozzle shape, such that there is a narrowing as the encapsulation region interfaces with the droplet channel.

Such an embodiment is beneficial, as the process of transporting the molecule to the encapsulation site is decoupled from the process of generating a droplet. This allows for a much more flexible system design, as droplets need only then be generated when a molecule is confirmed to be present, and once confirmed, there is no time limit on when the droplet need be formed, as the molecule will remain in place ready for encapsulation. This allows for droplet generation to be timed with other system level events, such as the need to synchronize with the current state of other droplets and their respective contents. In addition, this alleviates the need for generating a large number of ‘vacant’ droplets, which can complicate system level functions of the device when tracking single droplets is required, as system level resources will be consumed tracking droplets of no value.

In the embodiment whereby both entropic barriers are present (3703 and 3707), there is an added level of control, in that it allows for complete removal of the applied force between 3711 and 3713 after the molecule is in the encapsulation site, and physically separates said molecule from other molecules that may be in the 3704 channel that originate from 3711. In the preferred embodiment, a single-file flow of long nucleic molecules originate from 3711, sufficiently separated, such that they can be placed in the encapsulation site one-at-a-time via appropriately applied and timed external forces, and then when desired, encapsulated into droplets.

The encapsulation site should be appropriately sized for the desired droplet size to be generated. In some embodiments, the encapsulation site should have a sufficient volume to generate a 100 micron diameter droplet or larger, or 50 micron diameter droplet or larger, or 10 micron diameter droplet or larger, or 1 micron diameter droplet or larger.

A previous art demonstrated the use of nano-cracks to concentrate ions [Yu, 2015] at the point of droplet formation. However, in this prior art the physical mechanism of concentration, the encapsulated molecules, and application is different. The nano-crack serves to provide an ion concentration polarization (ICP) effect [Fu, 2018] in which an ion-selective nanochannel (nano-crack) allows for the generation of a charge depletion region from the balance of electrophoretic migration and electroosmotic flow, resulting in anions (the sample) to be concentrated at the boundary of the depletion region. Here, an entropic barrier prevents the transport of long polymer-like macro-molecules when in a deformable object coiled state through a mechanism described previously.

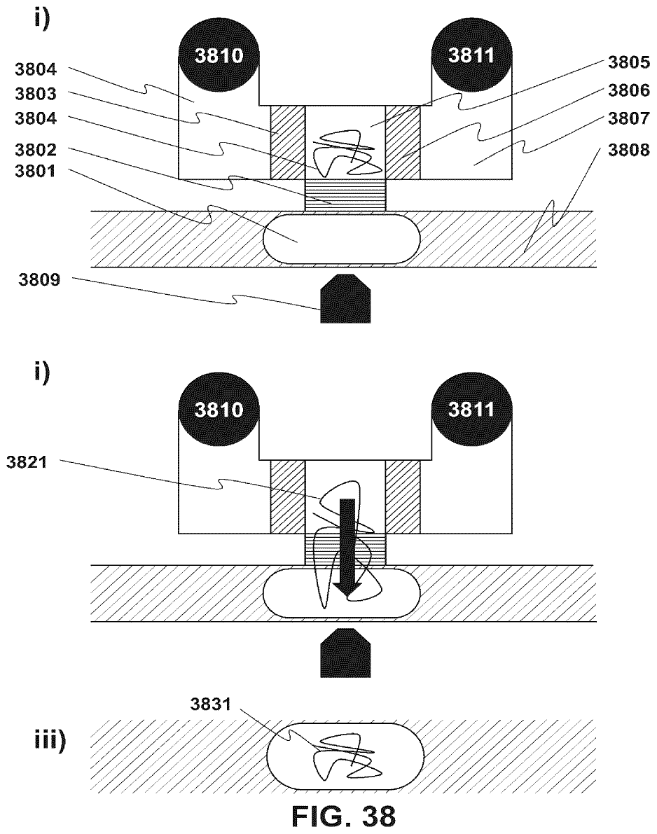

Another embodiment device and method shown in FIG. 38 is very similar in its operation to FIG. 37, except in this embodiment, a long nucleic acid molecule is encapsulated in a droplet by injecting the molecule into a pre-existing droplet. In this embodiment, an aqueous solution injecting region (“the encapsulation site”) 3805 is in fluidic connection with an oil droplet transport channel 3808 through an injector 3802, described in previous art [Weitz, 2009, U.S. Pat. No. 9,757,698]. To inject at least some of the solution in the injecting region, an electric field is applied from the injecting region across the droplet 3801 to an opposite terminal 3809. To enable the injection into droplets with a controlled occupancy of long nucleic acid molecules, a nucleic acid delivery cross-channel 3804 and 3807 are in fluidic connection with the injecting region 3805. There are multiple ways of operating such a device, depending on the configuration of the embodiment. There are two entropic barriers 3803 and 3806, of which both. or either of which, or none, may be present. If an entropic barrier is not present, then its respective nucleic acid delivery channel is in direct fluidic contact with the injection region. In the embodiment where-by entropic barrier 3806 is present, and 3803 is not, a long nucleic acid molecule 3804, originating from fluidic port 3810, is transported to the injecting region 3805, via an external force applied from 3810 to 3811, such that molecule is brought up to the barrier 3806, but the force is insufficient for the molecule to pass. Either by maintaining the same level of force, or less, or none, the molecule will remain at the injection region until it is desired to inject into a droplet 3801. When injection is to take place, an electric field is applied, here in this embodiment between fluidic connection point 3810 and the terminal 3809, and the solution from the injection region containing the molecule 3821 is then injected into a droplet. The result is a water-in-oil droplet containing the long nucleic acid molecule 3731.

Such an embodiment is beneficial, as the process of transporting the molecule to the injection region is decoupled from the process of injecting into a droplet. This allows for a much more flexible system design, as droplets need only then be injected when a molecule is confirmed to be present in the injection region, and once confirmed, there is no time limit on when the droplet need be formed, as the molecule will remain in place ready for injection. This allows for droplet injection to be timed with other system level events, such as the need to synchronize with the current state of other droplets and their respective contents.

The injection region should be appropriately sized for the desired amount of solution to be injected, and the desired size of the molecule contained within said solution. In some embodiments, the injection region should have volume to inject 100 picolitres of solution or more, or 10 picoliters or more, or 1 picoliter or more, or 100 femtoliters or more, or 10 femtoliters or more, or 1 femtoliter or more.

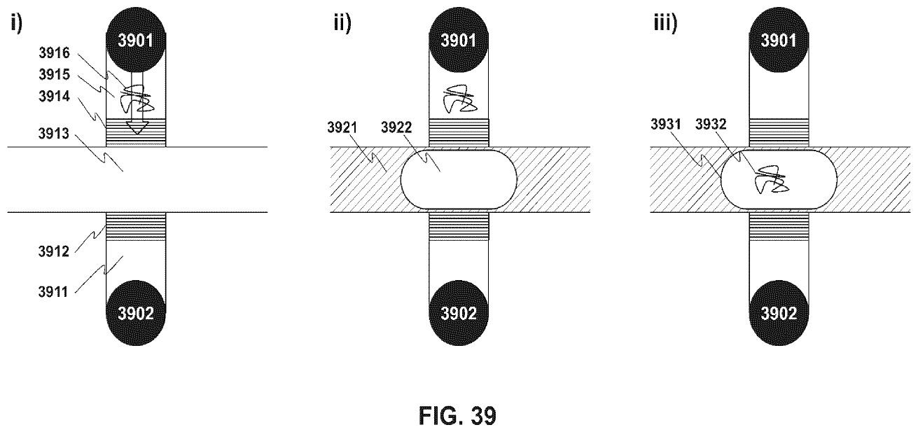

An additional embodiment device and method of injecting a long nucleic acid molecule is shown in FIG. 39. In this embodiment, the injector 3914 serves both as an injector and as an entropic barrier, such that a large nucleic acid molecule 3916 can be brought to the injector (“the encapsulation site”), but not over, via an appropriately small external force applied from 3901 towards the droplet transport channel 3913. In this particular embodiment, the applied force is an electric field applied between fluidic connection ports 3901 and 3902, where-by 3902 is similarly connected to the droplet transport channel via an entropic barrier (or injector) 3912. In order to allow charge transport carriers to flow from 3901 to 3902 such that long nucleic molecule 3916 can be brought up to the injector 3914, the droplet channel 3913 is filled with an aqueous solution. However, once the molecule has reached the injection point, oil 3921 can displace the water in the droplet transport channel, allowing for transport of a water-in-oil droplet 3922 to the vicinity of the injector. When desired, the long nucleic acid molecule 3932 can then be injected into the droplet 3931, via an applied electrical field from 3901 to 3902.

In all embodiments, fluorescent imaging can be employed to confirm the presence of the long nucleic acid molecule at the encapsulation site prior to encapsulation, and to confirm the molecule has been encapsulated. Furthermore, multiple encapsulation sites may be employed on a device, in which they can be triggered independently, or have a shared triggering mechanism. Electrodes if used may be solid or liquid in nature.

As described in the prior art [Weitz, 2009, U.S. Pat. No. 9,757,698], a solution can be injected into a droplet when the droplet is in the vicinity of the injector site. Such a device is extremely useful, but can present synchronization challenges when multiple droplets are desired to be injected into, each with a different injector, or combination of injectors. Such an operation would require independent firing control of the injectors, thus complicating the device's operation and design, or very precise control over the droplet's physical separation and speed such that the droplets can all simultaneously be in the proximity of their respective required injector, and then initiate simultaneous injection. Furthermore, unless the timing of the transport of the droplets past the injectors was extremely well managed, visual confirmation of successful alignment of the droplet and injection at the time of injection would be required.

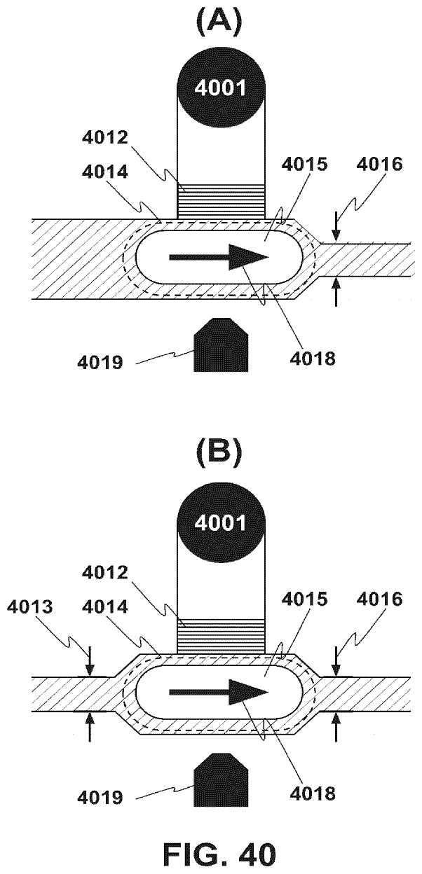

Here we describe an embodiment where-by a droplet can be trapped at the injector site such that it remains there until injection is desired, and then removed. It has been shown previously that transport of droplets in a channel can be blocked using restrictions in the channel [Fraden, 2007, U.S. Pat. No. 8,592,221], or limited expansions into which droplets can expand into [Boehm, 2008, U.S. Pat. No. 9,664,619]. Essentially both mechanisms are similar, in that they block the transit of the droplet (a deformable object) with an entropic barrier, which then requires the application of a sufficient force to overcome. FIG. 40(A) shows an embodiment device and method where-by a droplet 4015 is maintained in an injection region 4014 (which is also the capture site) in close proximity to an injector 4012 and counter electrode 4019. In one embodiment, the droplet is maintained at the injection region by presenting to the drop a restriction 4016 that has more confining dimension when compared to the injection region, such that there exists an external force 4018 that can be applied to the droplet that pushes the droplet against the barrier 4016, but insufficiently large that the droplet can deform and pass through. As such, with the application of this force, the droplet can be maintained at the injection region. In another embodiment shown in FIG. 40(B), there is a second restriction 4013, again that has a more confining dimension when compared to the injection region. The benefit of this second embodiment shown in FIG. 40(B), is that once the droplet has entered the injection region (capture site), it cannot exit in any direction unless there is a sufficiently applied force. (For clarity: the “restriction” for both 4016 and 4013 are defined from the perspective of a droplet in the injection region 4014, not necessarily outside of it. As such, there may be no apparent restriction to the droplet when entering the injection region, as is the case for an enlargement of the droplet channel at the injector site.)

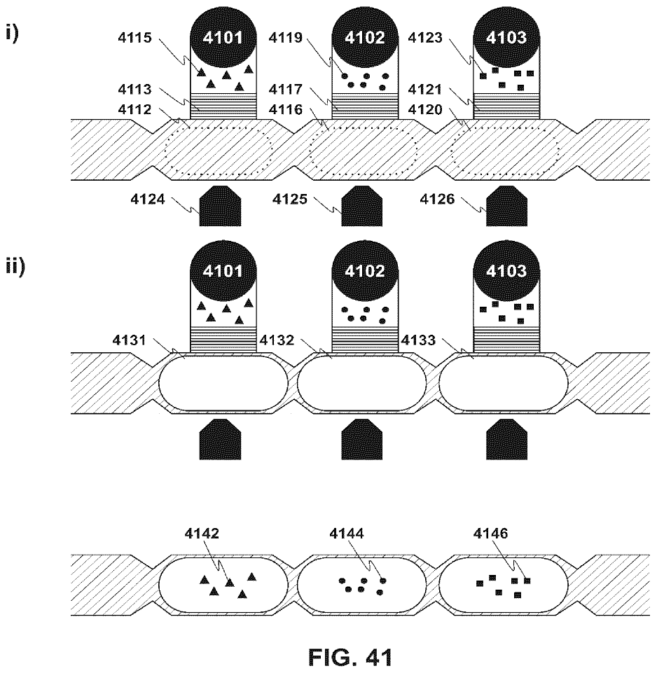

Such a droplet capture site is especially valuable when it is desired to have multiple injectors inject solution into multiple independent droplets simultaneously as shown in embodiment of FIG. 41. In such an embodiment, the injectors can all share the same electrodes, thus reducing the complexity of the device. Even with just one injector, such a droplet capture mechanism is of value, as it allows for blind injection into the droplet, as once a droplet has been captured, at any point afterwards, the control system can confidently control the injection of a solution into the droplet, and the droplets escape from the capture site, without a visual feedback system to monitor the events. By adding a capture site, these processes of droplet capture and droplet injection can be de-coupled. First the droplet is captured, and when it's desired for the system, the droplet injected.

The embodiment shown in FIG. 41 has 3 injectors (4113, 4117, 4121), each with their own respective droplet capture sites (4112, 4116, 4120), and each with their own solution composition to inject (4115, 4119, 4123). In this particular embodiment, they also each have their own independent counter electrode (4124, 4125, 4126), although in some embodiments the electrodes may be electrically joined, or they may all be the same shared electrode. After loading droplets (4131, 4132, 4133) into the respective droplet capture sites, the injectors can then be fired simultaneously, or independently at arbitrarily chosen times, resulting in droplets containing the desired solutions (4142, 4144, 4146).

In some embodiments, there may be shunting channels around the capture sites, such that once a droplet is captured, other droplets behind it in the droplet channel can go around the capture site.

### Associating Known Content in a Droplet With a Known Barcode

Currently there exists a variety of mechanisms that allow for ‘barcoding’ of droplets such that the contents of a droplet can be labeled as ‘unique’ from that of the other droplets [Regev, 2014, 2019/0127782], [Lan, 2017]. This is advantageous, as later on, if all the droplets, or a sub-set there-of, are to have their contents merged, as is common for most multiplexing applications, the unique barcode allows for maintaining separation of the sequencing datasets from the individual droplets. Once pooled, bodies with the same barcode can then be assumed to originate from the same droplet. However, since the barcodes are assigned randomly to droplets, what is not known is the relationship, if any, between the droplets. The only information provided by the barcode with these methods is the ability to identify droplet content as being different from each other. In certain applications it would be highly advantageous to associate a known unique barcode to a droplet, rather than a random, and blindly assigned, barcode to a droplet. For example, in situations where-by the maintaining a record of the relationship between barcodes is important, or tracking the origin of the contents within the droplet. In particular, when a long nucleic acid molecule is segmented into smaller child molecules in such way that the originating physical position within the parent molecule is known, and then the child is encapsulated into a droplet, a means to associate a known unique barcode with this droplet and its contents would be highly advantageous, as the association of this information with the droplet could then be maintained.

A prior art [Weitz, 2014, 2017/0029813] described a method of associating one or multiple tags (or barcodes) which track a droplet's history, thus enabling tracking the relationship between droplets once merged. Here we describe new methods and devices for tracking the precise relationship between individual droplets. For applications where it's critical that each individual droplet be differentiated from each other, and their relationship between each other known, each droplet needs to be associated with a unique, and known, barcode.

### Combinatorial Barcodes

In one embodiment device and method, a sample is encapsulated in a droplet under a controlled process such that the encapsulated sample in the droplet can be injected into with a unique combination of barcodes. In the preferred embodiment, the droplet contains a single long nucleic acid molecule, said molecule having been encapsulated in the droplet via one of the methods previously described. The droplet is transported in a droplet transport channel past a series of injectors, with each injector capable of injecting a solution containing a unique barcode, such that said droplet can then be injected with a known, and unique combination of unique barcodes. As each droplet passes the series of injectors, each droplet will receive a unique combination of injections, thus each droplet will then have a unique combination of barcodes inside. Once each droplet has been associated with a known unique combination of barcodes, the entire contents of the droplet can be amplified and prepared for sequencing. For example, previous work [Abate, 2015, 2017/0009274] describes a method of uniquely (but randomly) barcoding the entire contents of droplet such that after sequencing, the barcode can be determined.

In some cases, it may be advantageous to amplify the sample prior to adding the barcodes

### DNA Physical Map as a Unique Signature

In another embodiment device and method for tracking single droplets with a sample of interest relies on encapsulating one single long nucleic acid fragment with a known physical map profile in a droplet, where-by said molecule's physical map becomes the unique signature used to identify the droplet (eg: provides a unique pattern that can be used as an ID for tracking, much like a barcode). In some embodiments, the long nucleic acid molecule is itself the sample of interest. At a later point in time, after the droplet's long nucleic acid molecule has been sequenced, an in-silico physical map profile of the molecule of can be generated from the sequence data, which can then be matched back with the recorded physical map profile of the set of long nucleic acid molecules that were encapsulated in droplets and used as unique signatures. In some cases, the match will not be perfect because the assembled contigs are not continuous, or there are errors in the sequencing data, or there was a contamination or loss of nucleic acid in the droplet. In all cases, by using a best-fit match between sequenced data and recorded profiles, not only can the originating nucleic acid location be identified, but errors can also be corrected in the final sequence assembly.

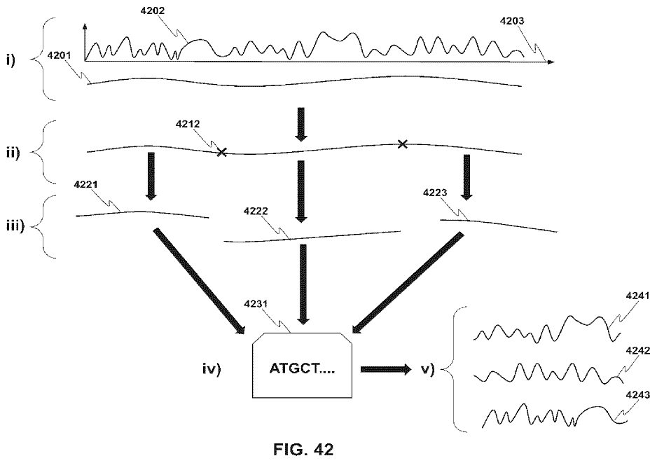

In one embodiment, shown in FIG. 42, a physical map 4202 is generated from the interrogation of a long nucleic acid parent molecule 4201. This parent molecule is then segmented by cleaving 4212 in a controlled manner as previously described for segregating parent molecules, or in a random manner, to generated three child molecules 4221, 4222, 4223, such that physical map is known for each child. Each child molecule is then encapsulated into a droplet. Using methods previously disclosed [Abate, 2015, 2017/0009274] a collection of droplets, each contain long fragments of DNA can be amplified and then sequenced 4231 using multiplexing techniques, such that sequencing contigs can be generated from each droplet individually. From these contigs, in-silico physical maps can be generated (4241, 4242, 4243) thus revealing the identity of the children. In some embodiments, the physical map of the child molecules are generated after the segmentation event to create the child from the parent. In some embodiments, the long nucleic acid molecule that provides the unique signature via its physical map is any long nucleic acid molecule, and not necessarily a child molecule.

The length of the long nucleic acid molecule to be used as a unique signature has no upper bounds, and can be long as single chromosomes <100 Mbp. The lower bounds molecule will depend on a variety of factors including the number of unique signatures required, the physical mapping method to be used for generating a unique signature, and the interrogation method used for reading the unique signature. For example, if only two unique signatures are required to uniquely track two droplets, then the length of the molecule need only be long enough to ensure with high confidence that two molecule's respective maps can be identified from each other. In most cases, the lower bound is approximately 1 kbp.

### Numbered Aspects of the Disclosure Herein

The disclosure is further elucidated through reference to the following numbered aspects of the embodiments herein. 1. A method, comprising: isolating an individual macromolecule; interrogating a physical characteristic of said macromolecule; and selectively performing a manipulation on least a region of said macromolecule. 2. The method of any of the above aspects, wherein the manipulation is a chemical manipulation. 3. The method of any of the above aspects, wherein the manipulation is a physical manipulation. 4. The method of any of the above aspects, wherein the physical characteristic is a physical map. 5. The method of any of the above aspects, wherein the physical map is generated by interrogating an elongated portion of the macromolecule's major axis. 6. The method of any of the above aspects, wherein the physical map is determined by interrogating at least two labeling bodies bound to the elongated portion of macromolecule. 7. The method of any of the above aspects, wherein the physical map correlates with the macromolecule's spatial genomic or structural content. 8. The method of any of the above aspects, wherein the physical map anti-correlates with the macromolecule's spatial genomic or structural content. 9. The method of any one of the above aspects, wherein the structural content includes DNA binding factors. 10. The method of any of the above aspects, wherein the selection of the region is at least in part informed by the comparative analysis of the physical map and a reference. 11. The method of any of the above aspects, wherein the region is one segment of at least two segments in the macromolecule. 12. The method of any of the above aspects, wherein the physical characteristic is interrogated on an elongated portion of the macromolecule's major axis. 13. The method of any of the above aspects, wherein the physical characteristic is located on a segment of the macromolecule that excludes the region. 14. The method of any of the above aspects, wherein the manipulation involves the delivery of at least one reagent in proximity to the region of said macromolecule, such that the at least one reagent can directly, or indirectly, enable, enhance, activate, or modify a reaction, binding, or cleaving within the region. 15. The method of any of the above aspects, wherein the reagent is delivered by positioning at least a portion of the macromolecule region in a channel of a fluidic device that transports the reagent. 16. The method of any of the above aspects wherein the reagent transport in the channel is by laminar flow. 17. The method of any of the above aspects, wherein the reagent is delivered by positioning at least a portion of the region in proximity to a reagent attached to a substrate via a cleavable linker, and releasing said reagent. 18. The method of any of the above aspects, wherein the substrate is a bead. 19. The method of any of the above aspects, wherein the substrate is a surface on fluidic device. 20. The method of any of the above aspects, wherein the substrate is a surface on a channel in a fluidic device. 21. The method of any of the above aspects, wherein the reagent is delivered by melting of a gelled material containing the reagent in proximity to the region. 22. The method of any of the above aspects, wherein the reagent is delivered by contacting at least a portion of the region to a drop of solution containing the reagent. 23. The method of any of the above aspects, wherein the solution drop is positioned by a dispensing system. 24. The method of any of the above aspects, wherein delivery of a reagent comprises photoactivating a photoactivatable pre-reagent in the vicinity of the reagent. 25. The method of any of the above aspects, wherein the reagent comprises an endonuclease. 26. The method of any of the above aspects, wherein the reagent comprises a nickase. 27. The method of any of the above aspects, wherein the reagent comprises a nucleic acid degrading component. 28. The method of any of the above aspects, wherein the reagent comprises a nucleic acid binding component. 29. The method of any of the above aspects, wherein the reagent comprises a degradation inhibitor. 30. The method of any of the above aspects, wherein the reagent comprises a nuclease inhibitor. 31. The method of any of the above aspects, wherein the reagent comprises an oligonucleotide. 32. The method of any of the above aspects, wherein the reagent comprises a recombinase. 33. The method of any of the above aspects, wherein the reagent comprises a primer. 34. The method of any of the above aspects, wherein the primer comprises a universal primer. 35. The method of any of the above aspects, wherein the universal primer comprises a barcode. 36. The method of any of the above aspects, wherein the reagent comprises a plurality of oligonucleotides. 37. The method of any of the above aspects, wherein the plurality of oligonucleotides comprises barcoded oligonucleotides. 38. The method of any of the above aspects, wherein the barcoded oligonucleotides indicate origin of the region. 39. The method of any of the above aspects, wherein the physical or chemical manipulation involves the delivery of at least one photon in proximity to the region of said macromolecule, such that the at least one photon can directly, or indirectly, enable, enhance, activate, or modify a reaction, binding, or cleaving event within the region. 40. The method of any of the above aspects wherein the photon un-cages an affinity group. 41. The method of any of the above aspects wherein the affinity group is connected to a binding body, said binding body bound to the macromolecule. 42. The method of any of the above aspects wherein the photon is used to cleave a photo-cleavable linker in close proximity to the region, and release a reagent. 43. The method of any of the above aspects wherein the reagent is released from a body. 44. The method of any of the above aspects wherein the reagent is released from a substrate 45. The method of any of the above aspects wherein the reagent is released from a surface on a fluidic device. 46. The method of any of the above aspects wherein the reagent is released from a surface of a fluidic channel within a fluidic device. 47. The method of any of the above aspects wherein the photon is used to photo-cleave the terminator of a reversable terminated nucleotide. 48. The method of any of the above aspects wherein the reversible terminated nucleotide is located on a the 3′ end of a primer hybridized to the macromolecule, and the macromolecule is a long nucleic acid molecule. 49. The method of any of the above aspects wherein the photon is used to photo-cleave nucleic acid within the region. 50. The method of any of the above aspects, wherein the physical or chemical manipulation involves the delivery of at least one contact probe in proximity to the region of said macromolecule, such that the at least one contact probe can directly, or indirectly, enable, enhance, activate, or modify a reaction, binding, or cleaving event within the region. 51. The method of any of the above aspects, wherein the contact probe is functionalized. 52. The method of any of the above aspects wherein the contact probe is an AFM. 53. The method of any of the above aspects wherein the contact probe delivers a reagent. 54. The method of any of the above aspects wherein the contact probe delivers a solution. 55. The method of any of the above aspects wherein the contact probe extracts the region. 56. The method of any of the above aspects, wherein the physical or chemical manipulation involves the delivery of at least one drop of solution in proximity to the region of said macromolecule, such that the at least one drop of solution can directly, or indirectly, enable, enhance, activate, or modify a reaction, binding, or cleaving event within the region. 57. The method of any of the above aspects wherein the at least one drop of solution is delivered by a dispenser. 58. The method of any of the above aspects wherein the at least one drop of solution is delivered by a contract probe. 59. The method of any of the above aspects, wherein the macromolecule comprises a polymer. 60. The method of any of the above aspects, wherein the macromolecule comprises a linear polymer. 61. The method of any of the above aspects, wherein the the macromolecule comprises a branched polymer. 62. The method of any of the above aspects, wherein the macromolecule comprises a nucleic acid. 63. The method of any of the above aspects, wherein the nucleic acid comprises a chromosome. 64. The method of any of the above aspects, wherein the nucleic acid is a branched nucleic acid. 65. The method of any of the above aspects, wherein the branched nucleic acid is generated through multiple displacement amplification. 66. The method of any of the above aspects, wherein the nucleic acid comprises a DNA stand reverse transcribed from an RNA template. 67. The method of any of the above aspects, wherein the nucleic acid comprises an RNA molecule. 68. The method of any of the above aspects, wherein the nucleic acid comprises a DNA stand reverse transcribed from an RNA template. 69. The method of any of the above aspects, wherein the nucleic acid comprises an RNA molecule. 70. The method of any of the above aspects, wherein the macromolecule comprises a long nucleic acid molecule. 71. The method of any of the above aspects, wherein the macromolecule is not cleaved prior to the physical or chemical manipulation. 72. The method of any of the above aspects, wherein the region comprises at least 10 bp. 73. The method of any of the above aspects, wherein the region comprises at least 50 bp. 74. The method of any of the above aspects, wherein the region comprises at least 100 bp. 75. The method of any of the above aspects, wherein the region comprises at least 500 bp. 76. The method of any of the above aspects, wherein the region comprises at least 1,000 bp. 77. The method of any of the above aspects, wherein the region comprises at least 5,000 bp. 78. The method of any of the above aspects, wherein the region comprises at least 10,000 bp. 79. The method of any of the above aspects, wherein the region comprises at least 100,000 bp. 80. The method of any of the above aspects, wherein the region comprises at least 1,000,000 bp. 81. The method of any of the above aspects, wherein isolating comprises extracting the individual macromolecule from a biological sample. 82. The method of any of the above aspects, wherein the biological sample comprises tissue from a healthy individual. 83. The method of any of the above aspects, wherein the biological sample comprises tissue form an individual seeking a diagnosis. 84. The method of any of the above aspects, wherein the biological sample comprises cancer tissue. 85. The method of any of the above aspects, wherein the biological sample comprises a cell. 86. The method of any of the above aspects, wherein the biological sample comprises no more than a single cell. 87. The method of any of the above aspects, wherein the biological sample comprises a viral particle. 88. The method of any of the above aspects, wherein the biological sample comprises a droplet. 89. The method of any of the above aspects, comprising analyzing the region. 90. The method of any of the above aspects, comprising providing a diagnosis. 91. The method of any of the above aspects, comprising selecting a treatment regimen. 92. The method of any of the above aspects, comprising administering the treatment regimen. 93. The method of any of the above aspects, wherein the macromolecule extracted from a sample retain at least some native three dimensional configuration. 94. The method of any of the above aspects, wherein extracting comprises removing the individual macromolecule from the biological sample while retaining at least some binding moieties bound to the individual macromolecule. 95. The method of any of the above aspects, wherein the binding moieties comprise chromatin constituents. 96. The method of any of the above aspects, wherein the binding moieties comprise histones. 97. The method of any of the above aspects, wherein the binding moieties comprise transcription factors. 98. The method of any of the above aspects, wherein the binding moieties comprise guide nucleic acids. 99. The method of any of the above aspects, wherein the binding moieties comprise nucleic acid protein complexes. 100. The method of any of the above aspects, wherein the binding moieties comprise CRISPR/CAS complexes. 101. The method of any of the above aspects, wherein isolating comprises positioning the macromolecule such that at least a portion of the region is elongated in a fluidic device. 102. The method of any of the above aspects, wherein isolating comprises positioning the macromolecule in a fluidic device such that it may be individually identified. 103. The method of any of the above aspects, wherein isolating comprises positioning the macromolecule such that it may be individually manipulated in a fluidic device. 104. The method of any of the above aspects, wherein isolating comprises positioning the nucleic acid in a fluidic device such that it may be subjected to a treatment that does not impact any other macromolecule. 105. The method of any of the above aspects, wherein interrogation comprises measuring an optical signal originating from at least one label body bound to the macromolecule. 106. The method of any of the above aspects, wherein the label body comprises an intercalating dye. 107. The method of any of the above aspects, wherein the physical characteristic is interrogated on at least one portion of the macromolecule in an elongated state, along the major axis. 108. The method of any of the above aspects, wherein the physical characteristic comprises macromolecular mass. 109. The method of any of the above aspects, wherein the physical characteristic comprises length along the major axis of the macromolecule. 110. The method of any of the above aspects, wherein the physical characteristic comprises spatial coordinates of the macromolecule. 111. The method of any of the above aspects, wherein the physical characteristic comprises spatial configuration of the macromolecule. 112. The method of any of the above aspects, wherein the physical characteristic comprises local melting temperature. 113. The method of any of the above aspects, wherein the physical characteristic comprises AT spatial density. 114. The method of any of the above aspects, wherein the physical characteristic comprises GC spatial density. 115. The method of any of the above aspects, wherein the physical characteristic comprises nucleic acid spatial density. 116. The method of any of the above aspects, wherein the physical characteristic comprises nucleic acid sequence spatial density. 117. The method of any of the above aspects, wherein the sequence is a recognition site. 118. The method of any of the above aspects, wherein the physical characteristic comprises nucleic acid sequence spatial pattern. 119. The method of any of the above aspects, wherein the sequence is a recognition site. 120. The method of any of the above aspects, wherein the physical characteristic comprises methylation spatial density. 121. The method of any of the above aspects, wherein the physical characteristic comprises histone occupancy. 122. The method of any of the above aspects, wherein the physical characteristic comprises transcription factor occupancy. 123. The method of any of the above aspects, wherein the physical characteristic comprises binding compound occupancy. 124. The method of any of the above aspects, wherein the physical characteristic comprises guide nucleic acid binding occupancy. 125. The method of any of the above aspects, wherein the physical characteristic comprises nucleic acid protein binding occupancy. 126. The method of any of the above aspects, wherein the physical characteristic comprises CRISPR/CAS complex binding occupancy. 127. The method of any of the above aspects, wherein the physical characteristic comprises phosphodiester bond integrity. 128. The method of any of the above aspects, wherein the physical characteristic comprises nucleobase integrity. 129. The method of any of the above aspects, wherein the physical characteristic comprises at least one ribose backbone lacking a nucleobase. 130. The method of any of the above aspects, wherein the physical characteristic comprises fluorescence. 131. The method of any of the above aspects, wherein the physical characteristic comprises antibody binding. 132. The method of any of the above aspects, wherein the manipulation comprises cleavage to release a segment from the nucleic acid. 133. The method of any of the above aspects, wherein the cleavage mechanism is photo-cleavage. 134. The method of any of the above aspects, wherein the cleavage mechanism is digestion with an enzyme. 135. The method of any of the above aspects where the enzyme is a restriction enzyme. 136. The method of any of the above aspects, comprising removing the segment spatially from remaining portions of the nucleic acid. The method of any of the above aspects, wherein the physical or chemical manipulation comprises amplification of the region of the nucleic acid. 137. The method of any of the above aspects, wherein the physical or chemical manipulation comprises binding at least one primer to the region of the nucleic acid. 138. The method of any of the above aspects, wherein the primers are universal primers. 139. The method of any of the above aspects, wherein the primers include a barcode. 140. The method of any of the above aspects, wherein the primers include a PCR binding site. 141. The method of any of the above aspects, wherein the physical or chemical manipulation comprises binding at least one barcode to the region of the nucleic acid. 142. The method of any of the above aspects, wherein the physical or chemical manipulation comprises delivery of a reagent to only the region. 143. The method of any of the above aspects, wherein the physical or chemical manipulation comprises delivery of a recombinase enzyme to enable loop formation. 144. The method of any of the above aspects, wherein the region is sequenced. 145. The method of any of the above aspects, wherein the region is encapsulated in a droplet. 146. The method of any of the above aspects, wherein the macromolecule is physically or chemically manipulated in a fluidic device. 147. The method of any of the above aspects, wherein the macromolecule is interrogated in a fluidic device. 148. The method of any of the above aspects, wherein at least a portion of the macromolecule is surrounded by a porous material. 149. The method of any of the above aspects wherein the porous material is a gelled material. 150. The method of any of the above aspects, wherein the fluidic device is a confined fluidic device. 151. The method of any of the above aspects, whereby the confined fluidic device includes at least one channel with a confining dimension <100 nm. 152. The method of any of the above aspects, wherein the fluidic device is an open fluidic device. 153. The method of any of the above aspects, wherein the open fluidic device comprises hydrophilic wells patterned on a hydrophobic surface. 154. The method of any of the above aspects, wherein the molecules are combed on the surface of the fluidic device. 155. A method than enables the physical partitioning of a long nucleic acid molecule into at least 2 partitions of nucleic acid, each partition occupying a separate entropic trap, connected by a connection portion of said molecule, in a fluidic device. 156. A method of any of the above aspects wherein the selection of partitions is at least in part informed by an interrogation of a physical map along an elongated portion of the molecule's major axis. 157. A method of any of the above aspects wherein at least a portion of the connecting portion of the molecule has its physical map interrogated. 158. A method of any of the above aspects wherein at least a portion of the long nucleic acid molecule has its physical map interrogated. 159. A method of any of the above aspects wherein the entropic traps have a least one dimension that can range from 50 nm to 50 microns. 160, A method of any of the above aspects wherein the entropic trap size is designed to contain a desired quantity of nucleic acid. 161. A method of any of the above aspects wherein at least one reagent is delivered to at least one partition in an entropic trap. 162. A method of any of the above aspects wherein at least one reagent is delivered to at least one connecting portion of the molecule. 163. A method of any of the above aspects wherein 2 partitions are physically separated from each other to form 2 segments by cleaving the connection portion of the molecule that connects them. 164. A method of any of the above aspects wherein the cleaving is enzymatic. 165. A method of any of the above aspects wherein the cleaving is photo-cleaving. 166. A method of any of the above aspects wherein information relating to the physical positional relationship of the segment within the originating long nucleic acid molecule is retained with said segment. 167. A method wherein a long nucleic acid parent molecule in a fluidic device can be segmented into child molecules in such a way that information relating child molecule's positional relationship along the parent molecule's major axis can be retained. 168. A method of any of the above aspects wherein the child boundaries within the parent are selected from an analysis of at least a portion of the parent molecule's physical map. 169. A method of any of the above aspects wherein at least one child's physical map is interrogated along the child's major axis. 170. A method of any of the above aspects wherein the information is retained with the child's physical map. 171. A method of any of the above aspects wherein the child is recognized by re-interrogating the child's physical map and comparing to a database physical maps. 172. A method of any of the above aspects wherein the child is recognized by sequencing the child, and generating an in-silico physical map from the sequence data, and comparing to a database of physical maps. 173. A method of any of the above aspects wherein the information is retained with physical isolation of the child from the other children and parent. 174. A method of any of the above aspects wherein the child is isolated in a droplet. 175. A method of any of the above aspects wherein the child is isolated in an entropic trap 176. A method of any of the above aspects wherein the child is isolated by extraction from the fluidic device. 177. A method of any of the above aspects wherein the information is used for sequencing assembly. 178. A method of any of the above aspects wherein the segmentation is by cleaving. 179. A method of any of the above aspects wherein the cleaving is enzymatic. 180. A method of any of the above aspects wherein the cleaving is photo-cleaving. 181. A method of any of the above aspects wherein the child is segmented from an elongated tail portion of a parent. 182. A method of any of the above aspects wherein at least one child is encapsulated in a droplet. 183. A method of any of the above aspects wherein at least one child is isolated in an entropic trap. 184. A method of any of the above aspects wherein the positional relationship is the numerical order along the parent molecule's major axis with respect to the other children. 185. A method of any of the above aspects wherein the positional relationship is the physical position within the parent from which the child was segmented. 186. A method of any of the above aspects wherein at least one child has at least one barcode associated with it. 187. A method of any of the above aspects wherein the barcode is bound to the child. 188. A method of any of the above aspects wherein the barcode is co-localized with the child in a droplet. 189. A method of concentrating at least one long nucleic acid molecule at a droplet encapsulation site with at least one entropic barrier. 190. A method of any of the above aspects wherein the mechanism for encapsulating the long nucleic acid molecule in a droplet, and the external force used for concentrating the long nucleic acid are de-coupled. 191. A method for claim X wherein the presence of the long nucleic acid molecule at the encapsulation site or its presence in the droplet can be confirmed by interrogation. 192. A method of any of the above aspects wherein the encapsulation method is droplet formation via pressure differential modulation between aqueous channel and an oil channel. 193. A method of any of the above aspects wherein the encapsulation method is injection of aqueous solution into an existing droplet in the droplet channel by an applied electrical field. 194. A method of any of the above aspects wherein the long nucleic acid molecule is concentrated at the encapsulation site of a droplet injector with an entropic barrier at the interface of the encapsulation site and droplet channel. 195. A method of any of the above aspects wherein the entropic barrier also serves as an injector. 196. A method of any of the above aspects wherein a solution capable of electrokinetic flow of charge carriers occupies the droplet channel during concentration. 197. A method of any of the above aspects wherein this solution is displaced with oil. 198. A method of maintaining a droplet at an injector site along a channel in a fluidic device with an entropic barrier or entropic trap. 199. A method of any of the above aspects wherein the injector can be triggered to inject a solution into a droplet maintained at the injector site at any time after confirmation of a droplet being present at the injector site. 200. A method of any of the above aspects wherein 2 or more droplets are simultaneously injected. 201. A method of any of the above aspects wherein for at least 2 injectors, the injector's positive electrodes are electrically connected and the injector's negative electrodes are electrically connected. 202. A method of generating a droplet containing at least one long nucleic acid molecule by trapping the said at least one molecule in an entropic trap, and then displacing the surrounding aqueous liquid with an oil liquid. 203. A method claim X of releasing said droplet from the entropic trap by modulating the trap's energy barrier to escape. 204. A method of associating information related to a droplet by encapsulating in said droplet a known combination of unique barcodes. 205. A method of any of the above aspects of confirming the association by sequencing the barcodes. 206. A method of any of the above aspects wherein the barcodes are encapsulated in the droplet by injection. 207. A method of any of the above aspects wherein the information known can include, but not limited to the following: droplet source, droplet contents, droplet history, droplet content history, droplet content origin. 208. A method of associating information related to a droplet by encapsulating in said droplet at least one long nucleic acid molecule with a known physical map. 209. A method of any of the above aspects of confirming the association by sequencing the at least one long nucleic acid molecule and reconstructing the physical map in-silico from the sequence data. 210. A method of any of the above aspects of confirming the association by interrogating the physical map of the at least one long nucleic acid molecule. 211. A method of any of the above aspects wherein the long nucleic acid molecule is encapsulated in the droplet by injection. 212. A method of any of the above aspects wherein the information known can include, but not limited to the following: droplet source, droplet contents, droplet history, droplet content history, droplet content origin. 213. A method of generating a positionally tagged nucleic acid library, comprising: positioning an long nucleic acid molecule; delivering a first reagent to a first elongated segment of the long nucleic acid molecule, wherein the first reagent comprises first positional tag information; delivering a second reagent to a second elongated segment of the long nucleic acid molecule, wherein the second reagent comprises second positional tag information; and wherein the first reagent is not delivered to the second region, and wherein the second reagent is not delivered to the first region. 214. The method of any of the above aspects, wherein the long nucleic acid molecule is not consumed pursuant to reagent delivery. 215. The method of any of the above aspects, comprising delivering a third reagent to a third elongated segment of the long nucleic acid molecule, wherein the third reagent comprises third positional tag information, and wherein the third segment of the long nucleic acid molecule overlaps the first segment and the second segment. 216. A positionally tagged nucleic acid library, comprising: a first set of library components sharing a first positional tag and a second set of library components sharing a second positional tag, wherein the first positional tag indicates an origin at a first segment of a nucleic acid molecule, and the second positional tag indicates an origin at a second segment of a nucleic acid molecule. 217. The library of any of the above aspects, wherein the first set of library components and the second set of library components are derived from a single common nucleic acid. 218. The library of any of the above aspects, wherein the single common nucleic acid is a chromosome. 219. The library of any of the above aspects, comprising a third set of library components sharing a third positional tag, wherein the third positional tag indicates an origin at a region overlapping at least a portion of the first segment and at least a portion of the second segment. 220. A method of selecting for a long nucleic acid molecule in a population of long nucleic acid molecules in a fluidic device, the method comprising interrogating the physical map of members of the population, and selecting a long nucleic acid molecule from the population based upon said molecule's physical map. 221. The method of any of the above aspects, wherein the population of long nucleic acid molecules comprises nucleic acids extracted from a sample. 222. The method of any of the above aspects, wherein the nucleic acids extracted from a sample retain native binding moieties. 223. The method of any of the above aspects, wherein the native binding moieties comprise proteins. 224. The method of any of the above aspects, wherein the proteins comprise chromatin constituents. 225. The method of any of the above aspects, wherein the proteins comprise histones. 226. The method of any of the above aspects, wherein the proteins comprise transcription factors. 227. The method of any of the above aspects, wherein the nucleic acids extracted from a sample retain at least some native three dimensional configuration. 228. The method of any of the above aspects, wherein the nucleic acids extracted from a sample are contacted to at least one labelling body prior to interrogation. 229. The method of any of the above aspects, wherein the labelling body comprises an intercalating agent. The method of any of the above aspects, wherein the labelling body differentially binds AT vs GC base pairs. 230. The method of any of the above aspects, wherein the labelling body differentially binds methylated nucleobases. 231. The method of any of the above aspects, wherein the labelling body comprises a protein. 232. The method of any of the above aspects, wherein the labelling body comprises a chromatin constituent. 233. The method of any of the above aspects, wherein the labelling body comprises a transcription factorThe method of any of the above aspects, wherein the labelling body comprises a nucleic acid binding protein. 234. The method of any of the above aspects, wherein the labelling body comprises a ligand. 235. The method of any of the above aspects, wherein the labelling body comprises an antibody. 236. The method of any of the above aspects, wherein the labelling body comprises an aptomer. 237. The method of any of the above aspects, wherein the labelling body comprises a guide nucleic acid. 238. The method of any of the above aspects, wherein the labelling body comprises a nucleic acid protein complex. 239. The method of any of the above aspects, wherein the labelling body comprises a CRISPR/CAS complex. 240. The method of any of the above aspects, wherein the molecule's physical map is interrogated on an elongated portion of the macromolecule's major axis, on which there is at least two labelling bodies. 241. The method of any of the above aspects, wherein the physical map comprises local AT basepair concentration. 242. The method of any of the above aspects, wherein the physical map comprises local nucleic acid density. 243. The method of any of the above aspects, wherein the physical map comprises local nucleic acid three dimensional structure. 244. The method of any of the above aspects, wherein the physical map comprises local density of a particular sequence. 245. The method of any of the above aspects, wherein the physical map comprises local frequency of a particular sequence. 246. The method of any of the above aspects, wherein the interrogation comprises a fluorescence monitor. 247. The method of any of the above aspects, wherein the interrogation detects protein binding. 248. The method of any of the above aspects, wherein the interrogation detects guide oligonucleotide binding. 249. The method of any of the above aspects, wherein the interrogation detects fluorescence. 250. The method of any of the above aspects, wherein the interrogation detects methylation status. 251. The method of any of the above aspects, wherein the interrogation detects local nucleic acid AT density. 252. The method of any of the above aspects, wherein the interrogation detects local nucleic acid density. 253. The method of any of the above aspects, wherein the interrogation detects nucleic acid three dimensional structure. 254. The method of any of the above aspects, wherein selecting a long nucleic acid molecule from the population based upon said molecule's physical map comprises selectively delivering a reagent to said molecule. 255. A method of any of the above aspects wherein the reagent is delivered by positioning at least a portion of the molecule in a channel of a fluidic device that transports the reagent. 256. A method of any of the above aspects wherein the reagent transport in the channel is by laminar flow. 257. A method of any of the above aspects wherein the reagent is delivered by a dispenser. 258. The method of any of the above aspects, wherein selecting a long nucleic acid molecule from the population based upon said molecule's physical map comprises selectively redirecting said molecule. 259. The method of any of the above aspects, wherein selecting a long nucleic acid molecule from the population based upon said molecule's physical map comprises selectively isolating said molecule. 260. A method of any of the above aspects, wherein the isolation comprises encapsulating the molecule in a droplet 261. A method of any of the above aspects, wherein the isolation comprises trapping the molecule in an entropic trap. 262. A method of any of the above aspects, wherein the isolation comprises extracting the molecule from the fluidic device. 263. The method of any of the above aspects, wherein selecting a long nucleic acid molecule from the population based upon said molecule's physical map comprises selectively exposing said molecule to photons. 264. The method of any of the above aspects, wherein selecting a long nucleic acid molecule from the population based upon said molecule's physical map comprises selectively exposing said molecule to a contact probe. 265. The method of any of the above aspects, wherein selecting a long nucleic acid molecule from the population based upon said molecule's physical map comprises selectively exposing said molecule to a drop of solution. 266. The method of X, wherein the drop of solution is delivered by a dispenser. 267. The method of any of the above aspects, wherein interrogating comprises elongating at least a portion of the long nucleic acid molecule in a confined fluidic device. 268. The method of any of the above aspects, wherein interrogating comprises combing at least a portion of the long nucleic acid molecule on an open fluidic device. 269. The method of any of the above aspects, wherein selecting a long nucleic acid molecule from the population based upon said molecule's physical map comprises selectively activating a pre-reagent locally at said molecule. 270. The method of any of the above aspects, wherein selectively activating a pre-reagent locally at the long nucleic acid molecule comprises directing photons locally at the nucleic acid molecule. 271. The method of any of the above aspects, wherein selectively activating a pre-reagent locally at the long nucleic acid molecule comprises delivering a liquid drop locally at the said molecule. 272. The method of any of the above aspects, wherein selecting a long nucleic acid molecule from the population based upon said molecule's physical map comprises comparing the molecule's physical map to a reference. 273. The method of any of the above aspects, wherein the reference comprises a predicted pattern. 274. The method of any of the above aspects, wherein the reference comprises an experimentally determined pattern. 275. The method of any of the above aspects, wherein the reference comprises a pattern assigned to at least one nucleic acid obtained from a database. 276. The method of any of the above aspects, wherein the reference comprises a pattern assigned to at least one genome obtained from a database. 277. The method of any of the above aspects, wherein the reference comprises a pattern assigned to at least one species obtained from a database. 278. The method of any of the above aspects, wherein the reference comprises a pattern generated from a simulation. 279. A method of any of the above aspects, wherein the simulation uses any of the following as inputs, including combinations there-of: sequence data, array data, 3D data, physical map data. 280. The method of any of the above aspects, wherein the reference comprises a consensus of at least two data sets. 281. A method of any of the above aspects, wherein the data sets can be any of the following: sequence data, array data, 3D data, physical map data. 282. The method of any of the above aspects, comprising selecting a long nucleic acid molecule having a physical map that matches the reference. 283. The method of any of the above aspects, comprising selecting a long nucleic acid molecule having a physical map that differs from the reference. 284. The method of any of the above aspects, wherein population of long nucleic acid molecules are extracted from a tumor. 285. The method of any of the above aspects, wherein population of long nucleic acid molecules are extracted from a patient suspected of having an infectious discase. 286. The method of any of the above aspects, wherein population of long nucleic acid molecules are extracted from a patient at risk of having a heritable disease. 287. The method of any of the above aspects, wherein population of long nucleic acid molecules are extracted from an environmental sample.

### REFERENCES

[Abate, 2015, 2017/0009274] Abate, A. R., Halliburton, J., Lan, F., & Sciambi, A. R. (filed 2015). SEQUENCING OF NUCLEIC ACIDS VIA BARCODING IN DISCRETE ENTITIES. US 2017/0009274 A1
[Abbyad, 2011] Abbyad, P., Dangla, R., Alexandrou, A., & Baroud, C. N. (2011). Rails and anchors: Guiding and trapping droplet microreactors in two dimensions. Lab on a Chip, 11(5), 813-821. website://doi.org/10.1039/c01c00104j
[Agresti, 2014, 2016/0060621] Agresti, J. J., Cooper, S., Karlin-Newmann, G., Heredia, N., & Lebofsky, R. (2014). 2016/0060621 DIGITAL PCR BARCODING,
[Åkerman, 1996] Åkerman, B., & Tuite, E. (1996). Single- and double-strand photocleavage of DNA by YO, YOYO and TOTO. Nucleic Acids Research, 24(6), 1080-1090. website://doi.org/10.1093/nar/24.6.1080
[Alberts, 2014] Alberts B, Bray D, Hopkin K, Johnson A, Lewis J, Raff M. Roberts K, Walter P (2014). Essential Cell Biology (Fourth ed.). New York, NY, USA: Garland Science. pp. 621-626. ISBN 978-0-8153-4454-4.
[Antonin, 2016] Antonin W, Neumann H (June 2016). “Chromosome condensation and decondensation during mitosis”. Current Opinion in Cell Biology. 40:15-22. doi:10.1016/j.ceb.2016.01.013. PMID 26895139.
[Baroud, 2010] Baroud, C. N., Gallaire, F., & Emi Dangla, R. (2010). Dynamics of microfluidic droplets. Lab on a Chip. website://doi.org/10.1039/c001191f
[Barrett, 1989, U.S. Pat. No. 5,252,743] Barrett, R. W., Pirrung, M. C., Stryer, L., Holmes, C. P., & Sundberg, S. A. (1989). U.S. Pat. No. 5,252,743 SPATIALLY-ADDRESSABLE IMMOBILIZATION OF ANTI-LIGANDS ON SURFACES.
[Barrett, 1989, U.S. Pat. No. 5,451,683] Barrett, R. W., Pirrung, M. C., Stryer, L., Holmes, C. P., & Sundberg, S. A. (1989). U.S. Pat. No. 5,451,683 SPATIALLY-ADDRESSABLE IMMOBILIZATION OF ANTI-LIGANDS ON SURFACES.
[Benner, 2005, U.S. Pat. No. 7,544,794] Benner, S. A. (2005). U.S. Pat. No. 7,544,794 METHOD FOR SEQUENCING DNA AND RNA BY SYNTHESIS.
[Benner, 2009, U.S. Pat. No. 8,034,923] Benner, S. A., Hutter, D., Leal, N. A., & Chen, F. (2009). U.S. Pat. No.8,034,923 REAGENTS FOR REVERSIBLY TERMINATING PRIMER EXTENSION.
[Bensimon, 1994, U.S. Pat. No. 5,840,862] Bensimon, D., Bensimon, A., & Heslot, F. (1994). U.S. Pat. No. 5,840,862PROCESS FOR ALIGNING, ADHERING AND STRETCHING NUCLEIC ACID STRANDS ON A SUPPORT SURFACE BY PASSAGE THROUGH A MENISCUS.
[Bensimon, 1995, U.S. Pat. No. 7,122,647] Bensimon, D., Bensimon, A., & Heslot, F. (1995). U.S. Pat. No. 7,122,647PROCESS FOR ALIGNING MACROMOLECULES BY PASSAGE OF A MENISCUS AND APPLICATIONS.
[Bensimon, 1995, WO 97/18326] Bensimon, A., Cremer, T., Kraus, J., & Lichter, P. (1995). WO 97/18326 ULTRAHIGH RESOLUTION COMPARATIVE NUCLEIC ACID HYBRIDIZATION TO COMBED DNA FIBERS.
[Bensimon, 1997, U.S. Pat. No. 7,368,234] 1 Bensimon, A., Bensimon, D., & Michalet, X. (1997). U.S. Pat. No. 7,368,234 PHYSICAL MAPPING METHOD USING MOLECULAR COMBING TECHNIQUE ALLOWING POSITIONING OF A GREAT NUMBER OF CLONES WITHIN A GENOME.
[Bensimon, 1999, WO 00/73503] Bensimon, A., Herrick, J., & Hyrien, O. (1999). WO 00/73503 THE USE OF THE COMBING PROCESS FOR THE IDENTIFICATION OF DNA ORIGINS OF REPLICATION.
[Boehm, 2008, U.S. Pat. No. 9,664,619] Boehm, C., Rowat, A. C., Koester, S., Agresti, J. J., & Weitz, D. A. (filed 2008). MICROFLUIDIC DEVICE FOR STORAGE AND WELL-DEFINED ARRANGEMENT OF DROPLETS. U.S. Pat. No. 9,664,619 B2
[Bonev, 2016] Bonev, Boyan; Cavalli, Giacomo (14 Oct. 2016), “Organization and function of the 3D genome”. Nature Reviews Genetics. 17 (11): 661-678. doi:10.1038/nrg.2016.112. ISSN 1471-0056.
[Caló, 2015] Caló, E., & Khutoryanskiy, V. V. (2015). Biomedical applications of hydrogels: A review of patents and commercial products. European Polymer Journal, 65, 252-267. website://doi.org/10.1016/j.curpolymj.2014.11.02
[Casavant, 2013] Casavant, B. P., Berthier, E., Theberge, A. B., Berthier, J., Montanez-Sauri, S. I., Bischel, L. L., . . . Beebe, D. J. (2013). Suspended microfluidics. Proceedings of the National Academy of Sciences of the United States of America, 110(25), 10111-10116. website://doi.org/10.1073/pnas.1302566110
[Chan, 1999, U.S. Pat. No. 6,696,022] Chan, E. Y., Gleich, L. C., & Wellman, P. S. (1999). METHODS AND APPARATUSES FOR STRETCHING POLYMERS., U.S. Pat. No. 6,696,022 B1
[Chan, 2006] Chan, T.-F., Ha, C., Phong, A., Cai, D., Wan, E., Leung, L., . . . Xiao, M. (2006). A simple DNA stretching method for fluorescence imaging of single DNA molecules. Nucleic Acids Research, 34(17). website://doi.org/10.1093/nar/gk1593
[Chen, 2011, U.S. Pat. No. 9,469,874] Chen, F., Zhang, T., Singh, K. K., Pennacchio, L. A., Froula, J. L., & Eng, K. S. (filed 2011), LONG-RANGE BARCODE LABELING-SEQUENCING. U.S. Pat. No. 9,469,874 B2
[Chen, 2013] Chen, F., Dong, M., Ge, M., Zhu, L., Ren, L., Liu, G., & Mu, R. (2013). The History and Advances of Reversible Terminators Used in New Generations of Sequencing Technology. Genomics, Proteomics and Bioinformatics, Vol. 11, pp. 34-40. website://doi.org/10.1016/j.gpb.2013.01.003
[Chen, 2016] Chen, G., Dong, J., Yuan, Y., Li, N., Huang, X., Cui, X., & Tang, Z. (2016). A general solution for opening double-stranded DNA for isothermal amplification. Scientific Reports, 6. website://doi.org/10.1038/srep34582
[Conti, 2003] Conti, et al. (2003) Current Protocols in Cytometry John Wiley & Sons, Inc
[Cook, 1999] Cook, G. P., Chen, T., Koppisch, A. T., & Greenberg, M. M. (1999). The effects of secondary structure and O2 on the formation of direct strand breaks upon UV irradiation of 5-bromodeoxyuridine-containing oligonucleotides. Chemistry and Biology, 6(7), 451-459. website://doi.org/10.1016/S1074-5521(99)80063-5
[Craighead, 1999, U.S. Pat. No. 6,635,163] Han, J., & Craighead, H. G. (filed 1999). ENTROPIC TRAPPING AND SIEVING OF MOLECULES. U.S. Pat. No. 6,635,163 B1
[Craighead, 2011, U.S. Pat. No. 9,926,552] Craighead, H. G., Topolancik, J., Tian, H. C., & Wallin, C. (2011). U.S. Pat. No. 9,926,552 MICROFLUIDIC DEVICE FOR EXTRACTING, ISOLATING, AND ANALYZING DNA FROM CELLS.
[Da Ros, 2005] Da Ros, T., Spalluto, G., Boutorine, A. S., Bensasson, R. V., & Prato, M. (2005). DNA-Photocleavage Agents. Current Pharmaceutical Design, 7(17). website://doi.org/10.2174/1381612013397140
[Dai, 2016] Dai, L., Renner, C. B., & Doyle, P. S. (2016). The polymer physics of single DNA confined in nanochannels. Advances in Colloid and Interface Science, Vol. 232, pp. 80-100. website://doi.org/10.1016/j.cis.2015.12.002
[Dean, 2001] Dean, F. B., Nelson, J. R., Giesler, T. L., & Lasken, R. S. (2001). Rapid amplification of plasmid and phage DNA using Phi29 DNA polymerase and multiply-primed rolling circle amplification. Genome Research, 11(6), 1095-1099. website://doi.org/10.1101/gr.180501
[Dean, 2002] Dean, F. B., Hosono, S., Fang, L., Wu, X., Faruqi, A. F., Bray-Ward, P., Sun, Z., Zong, Q., Du, Y., Du, J., Driscoll, M. D., Song, W., Kingsmore, S. F., Egholm, M., & Lasken, R. S. (2002). Comprehensive human genome amplification using multiple displacement amplification. Proceedings of the National Academy of Sciences of the United States of America, 99(8), 5261-5266. website://doi.org/10.1073/pnas.082089499
[deCarvalho, 2018] deCarvalho AC, Kim H, Poisson LM, Winn ME, Mueller C, Cherba D, et al. (May 2018). “Discordant inheritance of chromosomal and extrachromosomal DNA elements contributes to dynamic disease evolution in glioblastoma”. Nature Genetics. 50 (5): 708-717. doi:10.1038/s41588-018-0105-0. PMC 5934307. PMID 29686388.
[Desai, 2003] Desai, N., & Shankar, V. (2003). Single-strand-specific nucleases. FEMS Microbiology Reviews, 26. website://onlinelibrary.wiley.com/doi/10.1111/j.1574-6976.2003.tb00626.x/full
[Doddridge, 1998] Doddridge, Z. A., Warner, J. L., Cullis, P. M., & Jones, G. D. D. (1998). UV-induced strand break damage in single stranded bromodeoxyuridine-containing DNA oligonucleotides. Chemical Communications, 18, 1997-1998. website://doi.org/10.1039/a804416c
[Drmanac, 2018, 2018/0223358] Drmanac, R., Drmanac, S., Li, H., Xu, X., Callow, M. J., Eckhardt, L., . . . Ding, Q. (2017). 2018/0223358 STEPWISE SEQUENCING BY NON-LABELED REVERSIBLE TERMINATORS OR NATURAL NUCLEOTIDES.
[Drmanac, 2020] Drmanac, S., Callow, M., Chen, L., Zhou, P., Eckhardt, L., Xu, C., . . . Drmanac, R. (2020). CoolMPS TM: Advanced massively parallel sequencing using antibodies specific to each natural nucleobase. website://doi.org/10.1101/2020.02.19.953307
[Ebner, 2019] Ebner, A., Wildling, L., & Gruber, H. J. (2019). Functionalization of AFM tips and supports for molecular recognition force spectroscopy and recognition imaging. In Methods in Molecular Biology (Vol. 1886). website://doi org/10.1007/978-1-4939-8894-5_7
[Efcavitch, 2005, U.S. Pat. No. 8,071,755] Efcavitch, J. W., Siddiqi, S. M., Buzby, P. R., Mitchell, J., Krzymanska-Olejnik, E., Marappan, S., . . . Bowers, J. (2005). U.S. Pat. No. 8,071,755 NUCLEOTIDE ANALOGS.
[Efcavitch, 2013, U.S. Pat. No. 8,808,989] Efcavitch, J. W., & Siddiqi, S. M. (2013). U.S. Pat. No. 8,808,989 METHODS AND APPARATUS FOR SYNTHESIZING NUCLEOTIDES.
[Esteban, 1993] Esteban, J. A., Salas, M., & Blanco, L. (1993). Fidelity of 029 DNA polymerase. Comparison between protein-primed initiation and DNA polymerization. Journal of Biological Chemistry, 268(4), 2719-2726. website://doi.org/10.1016/s0021-9258(18)53833-3
[Fodor, 1989, 2003/0119011] Fodor, S. P., Solas, D. W., & Dower, W. J. (1989). 2003/0119011 ARRAYS FOR DETECTING NUCLEIC ACIDS.
[Fodor, 1989, U.S. Pat. No. 6,919,211] Fodor, S. P., Read, J. L., Stryer, L., & Pirrung, M. C. (1989). U.S. Pat. No. 6,919,211 POLYPEPTIDE ARRAYS.
[Fodor, 1989, U.S. Pat. No. 6,955,915] Fodor, S. P., Stryer, L., Pirrung, M. C., Read, J. L., & Hoeprich, P. D. (1989). U.S. Pat. No. 6,955,915 APPARATUS COMPRISING POLYMERS.
[Fraden, 2007, U.S. Pat. No. 8,592,221] Fraden, S., Boukellal, H., Selimovic, S., Rowat, A., Agresti, J., & Weitz, D. A. (filed 2007). MANIPULATION OF FLUIDS, FLUID COMPONENTS AND REACTIONS IN MICROFLUIDIC SYSTEMS. U.S. Pat. No. 8,592,221 B2
[Fu, 2018] Fu, L. M., Hou, H. H., Chiu, P. H., & Yang, R. J. (2018). Sample preconcentration from dilute solutions on micro/nanofluidic platforms: A review. Electrophoresis, Vol. 39, pp. 289-310. website://doi.org/10.1002/elps.201700340
[Gačanin, 2020] Gačanin, J., Synatschke, C. V., & Weil, T. (2020, January 11). Biomedical Applications of DNA-Based Hydrogels. Advanced Functional Materials, Vol. 30, p. 1906253. website://doi.org/10.1002/adfm.201906253
[Gersen, 2013] Gersen and Keagle, Principles of Cytogenetics, 3rd Ed 2013
[Gibb, 2012] Gibb, B., Silverstein, T. D., Finkelstein, I. J., & Greene, E. C. (2012). Single-stranded DNA curtains for real-time single-molecule visualization of protein-nucleic acid interactions. Analytical Chemistry, 84(18), 7607-7612. website://doi.org/10.1021/ac302117z
[Gole, 2013, 2016/0138013] Gole, J., & Zhang, K. (2013). 2016/0138013 SUBSTANTIALLY UNBIASED AMPLIFICATION OF GENOMES.
[Gueroui, 2002] Gueroui, Z., Place, C., Freyssingeas, E., & Berge, B. (2002). Observation by fluorescence microscopy of transcription on single combed DNA. Proceedings of the National Academy of Sciences of the United States of America, 99(9), 6005-6010. website://doi.org/10.1073/pnas.092561399
[Hammond, 2017] Hammond CM, Strømme CB, Huang H, Patel DJ, Groth A (March 2017). “Histone chaperone networks shaping chromatin function”. Nature Reviews. Molecular Cell Biology. 18 (3): 141-158. doi:10.1038/nrm.2016.159. PMC 5319910. PMID 28053344.
[Han, 2007] Han, J., Fu, J., & Schoch, R. B. (2007). Molecular sieving using nanofilters: Past, present and future. In Lab on a Chip (Vol. 8, Issue 1, pp. 23-33). website://doi.org/10.1039/b714128a
[Hilber, 2016] Hilber, W. (2016). Stimulus-active polymer actuators for next-generation microfluidic devices. Applied Physics A: Materials Science and Processing, 122(8). website://doi.org/10.1007/s00339-016-0258-6
[Hosono, 2003]
[Huang, 2015] Huang, L., Ma, F., Chapman, A., Lu, S., & Xie, X. S. (2015). Single-Cell Whole-Genome Amplification and Sequencing: Methodology and Applications. Annual Review of Genomics and Human Genetics, 16(1), 79-102. website://doi.org/10.1146/annurev-genom-090413-025352
[Hung, 2018]
[Hutchison, 2005]
[Hutchison, 2006]
[Hutter, 2010] Hutter, D., Kim, M. J., Karalkar, N., Leal, N. A., Chen, F., Guggenheim, E., . . . Benner, S. A. (2010). Labeled nucleoside triphosphates with reversibly terminating aminoalkoxyl groups. Nucleosides, Nucleotides and Nucleic Acids, 29(11-12), 879-895. website://doi.org/10.1080/15257770.2010.536191
[Ju, 2006] Ju, J., Hyun Kim, D., Bi, L., Meng, Q., Bai, X., Li, Z., . . . Turro, N. J. (2006). Four-color DNA sequencing by synthesis using cleavable fluorescent nucleotide reversible terminators. Retrieved from www.pnas.org/cgi/content/full/
[Juncker, 2002] Juncker, D., Schmid, H., Drechsler, U., Wolf, H., Wolf, M., Michel, B., . . . Delamarche, E. (2002). Autonomous microfluidic capillary system. Analytical Chemistry, 74(24), 6139-6144. website://doi.org/10.1021/ac0261449
[Kempfer, 2020] Kempfer, R., & Pombo, A. (2020). Methods for mapping 3D chromosome architecture. Nature Reviews Genetics, 21(4), 207-226. website://doi.org/10.1038/s41576-019-0195-2
[Knapp, 2011] Knapp, D. C., Serva, S., D'Onofrio, J., Keller, A., Lubys, A., Kurg, A., . . . Engels, J. W. (2011). Fluoride-cleavable, fluorescently labelled reversible terminators: Synthesis and use in primer extension. Chemistry-A European Journal, 17(10), 2903-2915. website://doi.org/10.1002/chem.201001952
[Kozlov, 2003, 7,259,258] Kozlov, I., Zhao, C., & Steinberg-Tatman, G. (2003). U.S. Pat. No. 7,259,258 METHODS OF ATTACHING BIOLOGICAL COMPOUNDS TO SOLID SUPPORTS USING TRIAZINE.
[Lan, 2016] Lan, F., Haliburton, J. R., Yuan, A., & Abate, A. R. (2016). Droplet barcoding for massively parallel single-molecule deep sequencing. Nature Communications, 7. website://doi.org/10.1038/ncomms11784
[Lan, 2017] Lan, F., Demaree, B., Ahmed, N., & Abate, A. R. (2017). Single-cell genome sequencing at ultra-high-throughput with microfluidic droplet barcoding. Nature Biotechnology, 35(7), 640-646. website://doi.org/10.1038/nbt.3880
[Larson, 1999]
[Lebofsky, 2003] Lebofsky, R., & Bensimon, A. (2003). Single DNA molecule analysis: Applications of molecular combing. Briefings in Functional Genomics and Proteomics, 1(4), 385-396. website://doi.org/10.1093/bfgp/1.4.385
[Leriche,2012] Leriche, G., Chisholm, L., & Wagner, A. (2012). Cleavable linkers in chemical biology. Bioorganic and Medicinal Chemistry, 20(2), 571-582. website://doi.org/10.1016/j.bmc.2011.07.048
[Li, 2019] Li, H., Liu, J., Li, K., & Liu, Y (2019). Piezoelectric micro-jet devices: A review. Sensors and Actuators, A: Physical, 297, 111552. website://doi.org/10.1016/j.sna.2019.111552
[Li, 2020] Li, C., Hite, Z., Warrick, J. W., Li, J., Geller, S. H., Trantow, V. G., . . . Beebe, D. J. (2020). Under oil open-channel microfluidics empowered by exclusive liquid repellency. Retrieved from website://advances.sciencemag.org/
[Lusic, 2006] Lusic, H., & Deiters, A. (2006). A new photocaging group for aromatic N-heterocycles. Synthesis, (13), 2147-2150. website://doi.org/10.1055/s-2006-942424
[Lusic, 2007] Lusic, H., Young, D. D., Lively, M. O., & Deiters, A. (2007). Photochemical DNA activation. Organic Letters, 9(10), 1903-1906. website://doi.org/10.1021/01070455u
[Maher, 2012] Maher CA, Wilson RK (2012). “Chromothripsis and Human Disease: Piecing Together the Shattering Process”. Cell. 148 (1-2): 29-32. doi:10.1016/j.cell.2012.01.006. PMC 3658123. PMID 22265399,
[Marcy, 2007]
[Mashid, 2018, U.S. Pat. No. 10,307,769] Mahshid, S., Ahamed, M. J., Berard, D., Amin, S., Sladek, R., Leslie, S. R., & Reisner, W. W. (2015). Development of a platform for single cell genomics using convex lens-induced confinement. Lab on a Chip, 15(14), 3013-3020. website://doi.org/10.1039/c51c00492f
[Mayo website] website://individualizedmedicineblog.mayoclinic.org/2019/04/16/rare-undiagnosed-discases-are-relatively-common/
[Mertes, 2011]
[Milton, 2018, Patent WO 2020/016606] Milton, J., Nayyar, S., Riedi, J., & Ogaki, R. (2018). WO 2020/016606 POLYNUCLEOTIDE SYNTHESIS METHOD, KIT AND SYSTEM.
[Mitelman, 2020] Mitelman Database of Chromosome Aberrations and Gene Fusions in Cancer (2019). Mitelman F, Johansson B and Mertens F (Eds.), website://mitelmandatabase.isb-cgc.org
[Moore, 2001] Moore, C. M., & Best, R. G. (2001). Chromosome preparation banding. Encyclopedia of Life Sciences.
[Mugele, 2005] Mugele, F., & Baret, J. C. (2005). Electrowetting: From basics to applications. Journal of Physics Condensed Matter, Vol. 17, pp. 705-774. website://doi.org/10.1088/0953-8984/17/28/R01
[Nathanson, 2014] Nathanson DA, Gini B, Mottahedeh J, Visnyei K, Koga T, Gomez G, et al. (January 2014). “Targeted therapy resistance mediated by dynamic regulation of extrachromosomal mutant EGFR DNA”. Science, 343 (6166): 72-6. Bibcode: 2014Sci343.72N. doi:10.1126/science.1241328. PMC 4049335. PMID 24310612.
[Nilsson, 2014] Nilsson, A. N., Emilsson, G., Nyberg, L. K., Noble, C., Stadler, L. S., Fritzsche, J., . . . Westerlund, F. (2014). Competitive binding-based optical DNA mapping for fast identification of bacteria-multi-ligand transfer matrix theory and experimental applications on Escherichia coli. Nucleic Acids Research, (1). website://doi.org/10.1093/nar/gku556
[NPAAC, 2013] Requirement for Cytogenetics Testing 3rd Edition 2013 NPAAC
[Pan, 2008]
[Perkins, 1995]
[Pieken, 1998, U.S. Pat. No. 6,737,236] Pieken, W., Hill, K., Eaton, B., McGee, D., Vagle, K., Gold, L., & Stephens, A. (1998). U.S. Pat. No. 6,737,236 BIOCONJUGATION OF MACROMOLECULES.
[Pieken, 1998, U.S. Pat. No. 7,427,678] Pieken, W., Wolter, A., Sebesta, D. P., Leuck, M., Latham-Timmons, H. A., Pilon, J., & Husar, G. M. (1998). U.S. Pat. No. 7,427,678 METHOD FOR IMMOBILIZING OLIGONUCLEOTIDES EMPLOYING THE CYCLOADDITION BIOCONJUGATION METHOD.
[Pihan, 2013] Pihan, G. a. (2013). Centrosome dysfunction contributes to chromosome instability, chromoanagenesis, and genome reprograming in cancer. Frontiers in Oncology, 3(November), 277. doi:10.3389/fonc.2013.00277
[Pirrung, 1996] Pirrung, M. C., & Huang, C. Y. (1996). A general method for the spatially defined immobilization of biomolecules on glass surfaces using “caged” biotin. Bioconjugate Chemistry, 7(3), 317-321. website://doi.org/10.1021/bc960013v
[Plawsky, 2009] Plawsky, J. L., Kim, J. K., & Schubert, E. F. (2009). Engineered nanoporous and nanostructured films. Materials Today, 12(6), 36-45. website://doi.org/10.1016/S1369-7021(09)70179-8
[Raghunathan, 2005]
[Ramsey, 2015, 10,106,848] Ramsey, J. M., & Menard, L. (2015). U.S. Pat. No. 10,106,848 NANOFLUIDIC DEVICES FOR THE RAPID WHOLE GENOMES AND RELATED SYSTEMS AND METHODS OF ANALYSIS. In US Patent.
[Rand, 2017] Rand, A. C., Jain, M., Eizenga, J. M., Musselman-Brown, A., Olsen, H. E., Akeson, M., & Paten, B. (2017). Mapping DNA methylation with high-throughput nanopore sequencing. Nature Methods, 14(4), 411-413. website://doi.org/10.1038/nmeth.4189
[Regev, 2014, 2019/0127782] Regev, A., Macosko, E. Z., McCarroll, S. A., Shalek, A. K., Basu, A., Ford, C. B., . . . Weitz, D. A. (filed 2014). DROPLET-BASED METHOD AND APPARATUS FOR COMPOSITE SINGLE-CELL NUCLEIC ACID ANALYSIS. US 2019/0127782 A1
[Reisner, 2005] Reisner, W. W., Morton, K. J., Richn, R., Wang, Y. M., Yu, Z., Rosen, M., . . . Austin, R. H. (2005). Statics and dynamics of single DNA molecules confined in nanochannels. Physical Review Letters, 94(19). website://doi.org/10.1103/PhysRevLett.94.196101
[Reisner, 2009] Reisner, W. W., Larsen, N. B., Flyvbjerg, H., Tegenfeldt, J. O., & Kristensen, A. (2009). Directed self-organization of single DNA molecules in a nanoslit via embedded nanopit arrays. Proceedings of the National Academy of Sciences of the United States of America. 106(1), 79-84.
[Richn, 2011]
[Rose, 2013, 2014/0272954] Rose, S., & Broeckel, U. (filed 2013). METHODS AND SYSTEMS FOR ELECTRONIC KARYOTYPING US 2014/0272954 A1
[Rush, 1985] Rush MG, Misra R (November 1985). “Extrachromosomal DNA in eucaryotes”. Plasmid. 14 (3): 177-91. doi:10.1016/0147-619X(85)90001-0. PMID 3912782.
[Samorodnitsky, 2015]
[Schenk, 2017]
[Schleyden, 1847] Schleyden, M. J. (1847). Microscopical researches into the accordance in the structure and growth of animals and plants.
[Schwartz, 1988, U.S. Pat. No. 6,147,198] Schwartz, D. C. (1988). U.S. Pat. No. 6,147,198 METHODS AND COMPOSITIONS FOR THE MANIPULATION AND CHARACTERIZATION OF INDIVIDUAL NUCLEIC ACID MOLECULES.
[Sharim, 2019] Sharim, H., Grunwald, A., Gabrieli, T., Michaeli, Y., Margalit, S., Torchinsky, D., . . . Ebenstein, Y. (2019). Long-read single-molecule maps of the functional methylome. Genome Research, 29(4), 646-656. website://doi.org/10.1101/gr.240739.118
[Sharpless, 2002, U.S. Pat. No. 7,375,234] Sharpless, K. B., Fokin, V., Rostovtsev, V. A., Green, L., & Himo, F. (2002). 7,375,234 COPPER-CATALYSED LIGATION OF AZDES AND ACETYLENES.
[Shen, 2013]
[Shiroguchi, 2012] Shiroguchi, K., Jia, T. Z., Sims, P. A., & Xie, X. S. (2012). Digital RNA sequencing minimizes sequence-dependent bias and amplification noise with optimized single-molecule barcodes. Proceedings of the National Academy of Sciences of the United States of America, 109(4), 1347-1352. website://doi.org/10.1073/pnas.1118018109
[Siddiqi, 2007, U.S. Pat. No. 7.956,171] Siddiqi, S. M. (2007). U.S. Pat. No. 7.956,171 NUCLEOTIDE ANALOGS.
[Smith, 2004, 2011/0059865] Smith, M. E., Sabot, A., Rasalongatovo, Isabelle, M., Sohna Sohna, J.-E. Horgan, A. M., & Swerdlow, H. P. (2004). 2011/0059865 MODIFIED MOLECULAR ARRAYS.
[Smith, 2010] Smith, A. M., Heisler, L. E., St. Onge, R. P., Farias-Hesson, E., Wallace, I. M., Bodeau, J., . . . Nislow, C. (2010). Highly-multiplexed barcode sequencing: An efficient method for parallel analysis of pooled samples. Nucleic Acids Research, 38(13). website://doi.org/10.1093/nar/gkq368
[Sounart, 2005] Sounart, T. L., Michalske, T. A., & Zavadil, K. R. (2005). Frequency-dependent electrostatic actuation in microfluidic MEMS. Journal of Microelectromechanical Systems, 14(1), 125-133. website://doi.org/10.1109/JMEMS.2004.839006
[Sounart, 2010] Sounart, T. L., Panchawagh, H. V, & Mahajan, R. L. (2010). Frequency-dependent stability of parallel-plate electrostatic actuators in conductive fluids. Applied Physics Letters, 96(20), 203505. website://doi.org/10.1063/1.3389491
[Stephens, 2011] Stephens PJ, Greenman CD, Fu B, et al. (2011). “Massive Genomic Rearrangement Acquired in a Single Catastrophic Event during Cancer Development”. Cell. 144 (1): 27-40. doi:10.1016/j.cell.2010.11.055. PMC 3065307. PMID 21215367.
[Stupi, 2011, U.S. Pat. No. 9,399,798] Stupi, B. P., Li, H., Wu, W., Hersh, M. N., Hertzog, D., Morris, S. E., & Metzker, M. L. (2011). U.S. Pat. No. 9,399,798 3′-OH UNBLOCKED, FAST PHOTOCLEAVABLE TERMINATING NUCLEOTIDES AND METHODS FOR NUCLEIC ACID SEQUENCING.
[Takahashi, 2017] Takahashi, Y., Kumatani, A., Shiku, H., & Matsue, T. (2017). Scanning Probe Microscopy for Nanoscale Electrochemical Imaging. Analytical Chemistry, 89(1), 342-357. website://doi.org/10.1021/acs.analchem.6b04355
[Tan, 2004] Tan, Y.-C., Fisher, J. S., Lee, A. I., Cristini, V., & Lee, A. P. (2004). Design of microfluidic channel geometries for the control of droplet volume, chemical concentration, and sorting f. Miniaturisation for Chemistry, Biology & Bioengineering. website://doi.org/10.1039/b403280m
[Tanyeri, 2011] Tanyeri, M., Ranka, M., Sittipolkul, N., & Schroeder, C. M. (2011). A microfluidic-based hydrodynamic trap: Design and implementation. Lab on a Chip, 11(10), 1786-1794.website://doi.org/10.1039/c01c00709a
[Tegenfeldt, 2008, U.S. Pat. No. 9,597,687] Tegenfeldt, J. O., Reisner, W. W., & Flyvbjerg, H. (Priority: 2008). U.S. Pat. No. 9,597,687 METHOD FOR THE MAPPING OF THE LOCAL AT/GC RATO ALONG DNA.
[Tegenfeldt, 2005] Wang, Y. M., Tegenfeldt, J. O., Reisner, W. W., Riehn, R., Guan, X. J., Guo, L., Golding. I., Cox, E. C., Sturm, J. C., & Austin, R. H. (2005). Single-molecule studies of repressor-DNA interactions show long-range interactions. Proceedings of the National Academy of Sciences of the United States of America, 102(28), 9796-9801. website://doi.org/10.1073/pnas.0502917102
[Tegenfeldt, 2009, U.S. Pat. No. 10,434,512] Tegenfeldt, J. O., Reisner, W. W., & Flyvbjerg, H. (filed 2009). METHOD FOR THE MAPPING OF THE LOCAL AT/GC RATIO ALONG DNA. U.S. Pat. No. 10,434,512 B2
[Telenius, 1992]
[Tøstesen, 2005] Tøstesen, E., Jerstad, G. I., & Hovig, E. (2005). Stitchprofiles.uio.no: Analysis of partly melted DNA conformations using stitch profiles. Nucleic Acids Research, 33(SUPPL. 2), 573-576. website://doi.org/10.1093/nar/gki424
[Turner, 2017] Turner KM, Deshpande V, Beyter D, Koga T, Rusert J, Lee C, et al. (March 2017). “Extrachromosomal oncogene amplification drives tumour evolution and genetic heterogeneity”. Nature. 543(7643): 122-125. Bibcode: 2017Natur.543.122T. doi:10.1038/nature21356. PMC 5334176. PMID 28178237
[Unger, 1999, U.S. Pat. No. 7,144,616] Unger, M. A., Chou, H.-P., Thorsen, T. A., Scherer, A., & Quake, S. R. (1999). U.S. Pat. No. 7,144,616 MICROFABRICATED ELASTOMERIC VALVE AND PUMP SYSTEMS (Vol. 520).
[Verhaak, 2019] Verhaak RG, Bafna V, Mischel PS (May 2019). “Extrachromosomal oncogene amplification in tumour pathogenesis and evolution”. Nature Reviews. Cancer. 19 (5): 283-288. doi:10.1038/s41568-019-0128-6. PMID 30872802.
[Volkmuth, 1992] Volkmuth, W. D., & Austin, R. H. (1992). DNA electrophoresis in microlithographic arrays. Nature, 358(6387), 600-602. website://doi.org/10.1038/358600a0
[Weissleder, 2013, 2017/0275669] Weissleder, R., Agasti, S. S., Peterson, V. M., & Ullal, A. (2013). 2017/0275669 METHODS, KITS, AND SYSTEMS FOR MULTIPLEXED DETECTION OF TARGET MOLECULES AND USES THEREOF.
[Weitz, 2009, U.S. Pat. No. 9,757,698] Weitz, D. A., Abate, A. R., Hung, T., & Marya, P. (filed 2009). FLUID INJECTION. U.S. Pat. No. 9,757,698 B2
[Weitz, 2014, 2017/0029813] Weitz, D. A., Iafrate, A. J., Zheng, Z., & Zhang, H. (filed 2014). METHODS AND SYSTEMS FOR DROPLET TAGGING AND AMPLIFICATION. US 2017/0029813 A1
[Whitesides, 2001] “Soft Lithography in Biology and Biochemistry,” by George M. Whitesides, Emanuele Ostuni, Shuichi Takayama, Xingyu Jiang and Donald E. Ingber, Annual Review of Biomedical Engineering, 2001, Vol. 3, pages 335-373;
[Wilson, 2002] Wilson, John (2002). Molecular biology of the cell: a problems approach. New York: Garland Science. ISBN 978-0-8153-3577-1.
[Wollhofen, 2017]
[Wu, 2007] Wu, J., Zhang, S., Meng, Q., Cao, H., Li, Z., Li, X., . . . Ju, J. (2007). 3′-O-modified nucleotides as reversible terminators for pyrosequencing. Proceedings of the National Academy of Sciences of the United States of America, 104(42), 16462-16467. website://doi.org/10.1073/pnas.0707495104
[Xia, 1998] “Soft Lithography,” by Younan Xia and George M. Whitesides, Annual Review of Material Science, 1998, Vol. 28, pages 153-184,
[Xiao, 2007] Xiao, M., Phong, A., Ha, C., Chan, T. F., Cai, D., Leung, L., . . . Kwok, P. Y. (2007). Rapid DNA mapping by fluorescent single molecule detection. Nucleic Acids Research, 35(3)
[Yasen, 2020] Yasen, A., Aini, A., Wang, H., Li, W., Zhang, C., Ran, B., . . . Wen, H. (2020). Progress and applications of single-cell sequencing techniques. Infection, Genetics and Evolution, Vol. 80. website://doi.org/10.1016/j.meegid.2020.104198
[Yoon, 2011]
[Yu, 2015] Yu, M., Hou, Y., Zhou, H., & Yao, S. (2015). An on-demand nanofluidic concentrator. Lab on a Chip, 15(6), 1524-1532. website://doi.org/10.1039/c41c01480d
[Zhang, 1992]
[Zhang, 2012] Zhang, M., Zhang, Y., Scheuring, C. F., Wu, C. C., Dong, J. J., & Zhang, H. Bin. (2012). Preparation of megabase-sized DNA from a variety of organisms using the nuclei method for advanced genomics research. Nature Protocols, 7(3), 467-478. website://doi.org/10.1038/nprot.2011.455
[Zhao, 2013] Zhao, Y.-P., & Wang, Y. (2013). Fundamentals and applications of electrowetting: A critical review. Reviews of Adhesion and Adhesives, 1(1), 114-174. website://doi.org/10.7569/RAA.2013.097304
[Zong, 2012]

### EXAMPLES

### Example 1: Fabrication of a Confined Fluidic Device and Operation

As an initial proof of concept, a model system for a confined fluidic device is developed in a geometry similar to the embodiment shown in FIG. 7(A) such that an elongated portion of the long nucleic acid molecule can be targeted with a flow of reagents. The intended device lateral geometries are first defined using a CAD software program such that contact photomasks can be specified for order from a mask vendor. Once obtained, a glass borofloat wafer 0.5 mm thick is spin coated with a layer of positive photoresist, and then prepared for exposure according to the resist manufactures instructions. Operating a mask aligner in contact mode, the resist on the wafer is exposed through the mask to UV light, after which the resist is developed according to the instructions and chemicals recommended by the manufacturer to remove the exposed resist and expose the glass surface in the elongation channel (0701) regions. The exposed glass is then etched in reactive ion etcher using a CHF 3 plasma to etch 50 nm deep. Here a very shallow etch is defined such that the vertical height provides a confining dimension to the long nucleic acid molecule, while the elongation channel width is 5 microns across. The resist is then removed in an oxygen ash plasma. The reagent channel (705) is manufactured in a similar manner, aligned to the elongation channel via fiducials. Here a reagent channel 1 micron deep is etched into the glass using an inductively coupled plasma (ICP) etcher with gas mixtures of SF6, NF3 and H2O. Notably, the reagent channel is 1 micron wide at the point in which it intersects with the elongation channel, as this width dimension defines the smallest ROI that can be selectively targeted on the elongated DNA.

With both the reagent and elongation channels now patterned in the surface of the glass substrate, the channels ends are connected to ports by sand blasting through the glass wafer using a metal shadow mask. The glass substrate is then thoroughly washed in a heated mixture of water, ammonia, and hydrogen peroxide to remove any remaining organic material and facilitate particle removal from the surface. Finally, the fluidic device is completed by plasma assisted fusion bonding the patterned glass wafer to a non-patterned glass wafer at 400 C, and then annealed in an oven at 650 C. Once cooled, the wafer is then diced into individual chips, and the fluidic ports are interfaced with a plastic manifold allowing for luer lock connections to all inlet and outlet ports.

The confined fluidic device is designed to operate such that a syringe pump can flow a reagent solution through the reagent channel that intersects with the elongation channel, with laminar flow maintaining the reagents within the reagent channel

### Example 2: Fabrication of an Open Fluidic Device

As an initial proof of concept, a model system for an open fluidic device is developed in a geometry similar to the embodiment shown in FIG. 16. The intended device lateral geometries are first defined using a CAD software program such that contact photomasks can be specified for order from a mask vendor. Once obtained, a glass borofloat wafer 0.5 mm thick is coated with 20 nm of chrome and 100 nm of gold evaporated over the surface of substrate. Next, a layer of positive photoresist is spin coated over the surface, and then prepared for exposure according to the resist manufactures instructions. Operating a mask aligner in contact mode, the resist on the wafer is exposed through the mask to UV light, after which the resist is developed according to the instructions and chemicals recommended by the manufacturer to remove the exposed resist and expose the gold film surface where the wells will be formed. The glass is submerged in a gold and chromium etchant to remove the metal in the wells, followed by an oxygen ash to remove the resist. The glass is submerged in a liquid glass etchant that contains HF and allowed to etch the glass to a depth of 2 microns. The HF wet etch is isotropic, so the wells grow in size by 2 microns in all directions after etch. In this example the 3 micron squares are patterned with 6 micron spacing, and so after removal of the metal hard masks, the final well size at the surface is 7 microns, with a 2 micron spacing. The etched glass substrate is then thoroughly washed in a heated mixture of water, ammonia, and hydrogen peroxide to remove any remaining organic material and facilitate particle removal from the surface.

Next, the top glass surface is treated with a hydrophobic silane monolayer to silanize the surface. This will both allow for DNA adhesion during the combing process, and for containment of solution within the wells. Silane treatment is performed by contact printing against a PDMS film that was previously submerged in a solvent of silane molecules, thus transferring the molecules to the regions between the wells via direct physical contact. The contact printing does not modify the wells, which due to their depressed topography, retain the glass's hydrophilic nature. After a 50 C anneal for 1 hour, the device is ready for use. As designed in this example, the wells are 7 microns in sized, spaced by 2 micron. Assuming the long nucleic acid molecules will be 100% stretched when combed on the surface, each well then will have approximately 23 kbp of nucleic acid spanned across the well, which then represents the smallest unit of ROI that can be targeted with the device, but a length scale that is easily accommodated with long range PCR. Furthermore, the well volume is able to contain approximately 1 picoliter of dispensed solution, which is achievable with piezoelectric micro-jet devices.

### Example 3: Fluorescent Control Instrument for Interrogating Physical Map

A control instrument consists of a Nikon Ti2-E inverted microscope with a CFI Apo TIRF 60XC oil immersion objective and a QHYCCD QHY294M-PRO Camera with a Sony IMX492 sensor operated in 2×2 binning mode. The instrument has a field of view of 190 μm×250 μm, allowing 750 kb of fully stretched DNA to be visualized with an optical resolution of 500 bp, allowing a simultaneous view of multiple regulatory element binding sites (6-20 bp, 2 nm-6.6 nm), intro-exons (100s bp-1000s bps-33-330 nm), gene locus/ORF (1000s bp-330 nm-3 microns). Larger features such as numerical abnormalities on the range of 1 MB-10 MB require either multiple stitched frames using an XY stage to move the device relative to the objective between frames or a combination of imaging and nucleic acid manipulation to translate the nucleic acid relative to the objective. Through objective TIRF illumination is performed with a 488 nm laser. Alternately widefield brightfield illumination is used to illuminate the fluidic device and center it in the field of view.

The control computer further controls a bank of solenoids that modulate pressure driven flow of fluid into the nanofluidic device of Example 1.

Human genomic DNA is isolated from blood samples by embedding purified nuclei in low melting point agarose plugs [Zhang, 2012]. The sample is electroeluted into low salt denaturing buffer (0.1×TBE, 20 mM NaCl, 2%-mercaptoethanol) with YOYO-1 at a ratio of 1 dye per 10 nucleotide pairs and incubated at 18 C overnight. The sample is diluted 1:1 with formamide with minimal manipulation and heated to 31 C for 10 minutes [Tegenfeldt, 2009, U.S. Pat. No. 10,434,512] before quenching on ice. The sample is immediately added to the device which is kept at temperature of 16-19 C.

The device is brought into focus using brightfield imaging and then the instrument is switched to TIRF fluorescence mode. DNA is gently flowed into the analyte elongation channel, at which point focus tracking is enabled and automated analysis is initiated. A control algorithm flows in DNA, stops flow, waits for DNA to settle, acquires 512 consecutive images of the DNA. Images are post-processed to isolate individual DNA molecules and align each of the individual frames to a consensus frame. DNA that photocleaves during the imaging process is discarded. The final consensus image is background adjusted and reduced to an 8-bit trace as a function of DNA position along the channel, and this is used as the physical map that estimates local GC content.

The physical map is compared with a pre-computed reference physical maps that are derived from sequences of the human genome assembly GRCh37 analyzed for melting state by the method of [Tøstesen, 2005]. Reference map segments are sampled at intervals corresponding to one pixel of detected image and each pixel worth of GC ratio information is normalized as a signed 8 bit integer, where—128 represents 100% AT, 127 represents 100% GC. The reference map is pre-computed for a variety (up to 20) DNA stretch ratios, so the same sequence is present multiple times. Observed maps are compared with the physical map references in two steps, first each molecule is artificially segmented into 32 pixel segments starting every other pixel. This corresponds to approximately 8-13 kbp depending on DNA stretch. The dot product of each segment and a 32 pixel tile of the reference map segments is computed. The top 4k matches are passed to the second stage, which repeats the dot product on neighboring regions in both the map and the sample and scores them with a Smith-Waterman algorithm to permit local insertions and deletions. Detection cutoffs are determined empirically.

In this example, only molecules that do not match to the known reference maps are selected for further manipulation and the control algorithm repeats this flow/imaging/selection process until stopped manually.

### Example 4: Creating a Sequencing Library of 60-80 kbp Contigs of a Targeted Region of a Single Long DNA Molecule Selected From Native Genomic DNA

The instrument and sample of Example 3 are used with a device that builds upon the device of Example 1. The device further contains an array of nanopit entropic traps downstream of the elongation channel. As per Example 3, long DNA molecules (megabase length) are repeatedly loaded, interrogated and compared with a reference map in order to select the region of interest. In the present example the target ROI is any molecule matching the DYZ3 locus of the human Y chromosome which contains the centromere and contains a 300 kbp region comprised of 5.8 kb repeats. When a molecule is found that matches that region, further manipulation is performed to flow the selected molecule over the array of nanopits (FIG. 29).

The nanopits are located within a nanoslit of depth 110 nm. The pits are 400 nm deep (510 nm between pit bottom and glass) and square with an edge length of 400 nm. The grid of pits is square with 2 μm spacing between pits. The pits each confine approximately 50 kb of DNA, while approximately 30 kb of DNA stretches between them.

The instrument relocates its field of view to follow the molecule into the nanopits and the molecule is allowed to relax for 10 minutes to equalize the amount of DNA in each pit. As per Example 3, a series of images are recorded of the molecule and the regions of DNA spanning the pits are processed to create a physical map of the molecule writhing through 2-D instead along a 1-D channel. The mean path of the DNA backbone is estimated using Gaussian process regression and the physical map is computed along the contour of the DNA. The map is compared with the original images of the ROI in order to map the pose of the molecule on and around the nanopits with the original pose of the molecule in the elongation channel. Matching is accomplished by computing scale-invariant moments of the nanopit physical maps and matching them against the same moments computed on sliding windows along the physical map of the elongated molecule.

The instrument photocleaves the DNA by first flowing a photocleaving buffer over the DNA, which is otherwise identical to the loading buffer but which omits-mercaptoethanol. A brightfield image of the nanopits is taken and the grid is located computationally. 488 nm light is then directed specifically to the regions of the device between the nanopits by illuminating a digital micromirror device (DMD) placed at a conjugate plane to the sample and relayed through the primary microscope objective in an epi-illumination configuration. The DMD is programmed to match the 488 nm light to the regions between the nanopits.

The result of the photocleaving is a cluster of DNA fragments in neighboring pits. Due to the regular construction of the pits, the DNA fragments are of uniform length, here between 60 and 80 kb. The physical mapping results from both the original elongated pose and the nanochannel pose at time of cleaving are saved to the control computer and the DNA is eluted at high flow rate and captured. The molecules are barcoded, amplified, sequenced and assembled into contigs using the method of Lan et. Al. 2016. The contigs are used to create a reference physical map, which is compared with the saved physical maps and used to assemble the contigs into a larger contig, or fragments thereof if some of the eluted molecules are not successfully sequenced.

### Example 5: Capture ROI With Entropic Device

The instrument, sample and device of Example 4 are used, but a telomere staining probe TelC-Cy5 (PNA Bio Inc) is added to the sample at a final concentration of 200 nm prior to incubation at 31 C.

Molecules are sequentially loaded and interrogated as per Example 3, with the addition of a second image through a Cy5 channel using 635 nm laser excitation through objective TIRF. The physical map selection criteria is simply the presence of a Cy5 signal, which indicates that a telomere from some chromosome was present. A secondary physical map of YOYO-1 fluorescence is also taken, but not used for the ROI selection.

Telomeric DNA is selectively moved to the nanopit array and gently manipulated back and forth using finely controlled fluid flow to place the telomere end cleanly in a nanopit. The inter-pit regions are mapped back to the elongated molecule YOYO-1 physical map for reference. The region 150 kb-550 kb away from the telomer end is identified by counting nanopit intervals and selecting the 3rd through 9th nanopits, where the 1st nanopit contains the TelC-Cy5 labeled telomer, The remaining DNA is photocleaved using the method of Example 4, but in this case all DNA not in the selected nanopits is irradiated and cleaved, regardless of whether it is in a nanopit or between nanopits. The cleaved fragments are washed away with gentle flow. The long ROI is eluted from the device by strong flow.

### Example 6: Targeting ROIs of a DNA With Specific MDA Primers in a Confined Fluidic Device

The following example uses the confined microfluidic chip described in Example #1, along with the DNA sample preparation and interrogation instrument described in Example #3. In this example a 500 kbp long molecule prepared with a physical map as described in Example #2 is in fully elongated state in the elongation channel such that an ROI can identified by the interrogation system, per the process previously described in Example #3. In this example, the ROI of interest is the translocation event that forms the chimeric gene BCR/ABL on chromosome 22. Here, the physical map allows break-point resolution of <1 kbp, and it is desired to selectively sequence the ROI defined as the break-point plus 25 kbp in either direction such that both gene fragments and any regulatory content up stream or downstream can also be captured. A 50 kbp ROI corresponds to approximately 15 microns in length at 100% elongation.

The reagent channel contains a denaturing alkaline solution along with an MDA universal primer mix. Here, the MDA universal primer consists of a PCR binding site at the 5′ end, followed by a 6 base random sequence (eg: 6′-NNNNNN-3′) which is the universal primer. The reagent channel is first primed with the MDA primer solution. Once primed, the flow rate is stopped, and elongated molecule is transported in the elongation channel, through the intersection region until the interrogation instrument registers the alignment of the start of the ROI boundary via the molecule's physical map with the reagent channel, at which point the reagent flow re-starts. The molecule continues its transport through the intersection as the 15 micron length ROI is exposed to flowing denaturing solution and primers. Confirmation of de-naturing of the molecule, thus allowing for primer binding, is achieved by loss of the physical map within the regent channel due to shedding of the intercalating dye. Once the 15 micron ROI has been exposed, the reagent flow is ceased, and the remainder of the molecule transported through the intersection region, and collected at the channel outlet to perform MDA followed by targeted PCR amplification off-device.

### Example 7: Targeting ROIs of DNA With Magnetic Beads on an Open Fluidic Device With Dispenser

The following examples used an open fluidic chip as described in Example 2, consisting of 7 micron square hydrophilic wells, 2 microns deep, patterned at 2 micron spacing on a glass substrate. Long nucleic acid molecules are prepared with a YOYO melt-map as described previously by [Tegenfeldt, 2008, patent], and then combed on the surface by dispensing the solution of DNA onto the glass substrate while maintained at a 45 degree angle, allowing the trailing meniscus of the solution droplet to attach the molecule ends to the hydrophobic glass top surface.

When the surface has completely dried, the open fluidic device is then transferred to the interrogation system (previously described in Example 3) and the molecule physical maps are interrogated. In this particular example, the interrogation system identifies a translocation breakpoint in the physical map of a single 250 kbp long nucleic acid molecule as the ROI, and registers the physical x-y location of the ROI on the surface of the device. A 1 picoliter drop of DNA-binding magnetic beads solution is dispensed in the well over which the ROI is suspended, using the previously determined x-y location., Next, the nucleic acid molecule is photo cleaved on either side of the well, such that the desired segment containing the translocation is now an isolated nucleic acid fragment approximately 23 kbp in length suspended in the solution of dna-binding magnetic beads within the well. After sufficient time to bind, a pipette dispensing and extraction system dispenses 1 uL of solution over the sample to re-suspend the DNA in the larger drop, and then extracts the 1 uL drop of solution from the surface of the open fluidic chip via suction. The ROI is isolated from any non-ROI DNA that may have been collected with a magnetic field.

### Example 8: Generating a Droplet With a Single Long Nucleic Acid Molecule With Physical Map Signature

As an initial proof of concept, a model system for a confined fluidic device is developed in a geometry similar to the embodiment shown in FIG. 37. In this particular example, the droplet generating channel (3708), the droplet channel (3701) and the nucleic acid delivery channels (3704, 3706) are all 50 microns wide, and 50 microns deep. In addition, in this example, the entropic barrier 3703 is not present, only the entropic barrier 3707 is defined in this device, such that the channel 3704 and the channel 3708 are in direct fluidic contact with each other. The entropic barrier (3707) has a constricting vertical dimension of 50 nm, and is 20 microns in length.

A 250 kbp long nucleic acid molecule in a buffer solution that has previously had its physical map interrogated, enters the device through the inlet port (3711) via an applied electric field of 10V applied from 3713 to 3711, flowing the molecule via the electro kinetic force to the encapsulation region (3702), where the molecule is pushed up against the entropic barrier (3707), but not does not pass over. Fluorescent imaging using the interrogation system described in Example 3 confirms the presence of the molecule in the encapsulation region. The applied voltage is decreased to 2 volts, so as to relax the molecule, but maintain its physical position within the encapsulation region, and adjacent to the entropic barrier.

At the desired time, a droplet is formed that encapsulates the long nucleic acid molecule. Droplet generation is achieved via removal of the applied voltage, and an applied pressure spike from fluidic connection 3712 into the droplet channel 3701 such that the aqueous solution in the encapsulation region is injected into the oil droplet channel, where-by a droplet is formed by interaction of the immiscible fluids. The droplet size is controlled by the duration and intensity of the pressure spike. Fluorescent monitoring with the interrogation system is used to confirm transit of the molecule into the droplet. In this example, the entire volume of the encapsulation region is used to generate a droplet approximately 200 pico-liters. The droplet is taken off device to undergo amplification and sequencing the per the protocol previously outlined by [Abate, 2015, 2017/0009274], from which sequence contigs for the droplet are generated. From these sequence contigs, an in silico physical map can be generated, and compared to the physical map interrogated from the long nucleic acid molecule originally encapsulated in the droplet, thus confirming the identify of the sequenced droplet.

We claim:

1. A method, comprising: isolating an individual macromolecule; interrogating a physical characteristic of said macromolecule; and selectively performing a manipulation on least a region of said macromolecule.

2. The method of claim 1, wherein the manipulation is a chemical manipulation.

3. The method of claim 1, wherein the manipulation is a physical manipulation.

4. The method of claim 1, wherein the physical characteristic is a physical map.

5. The method of claim 4, wherein the physical map is interrogated on an elongated portion of the macromolecule's major axis.

6. The method of claim 4, wherein the physical map comprises at least two labeling bodies bound to the elongated portion of macromolecule's major axis.

7. The method of claim 4, wherein the physical map correlates with the macromolecule's spatial genomic or spatial structural content.

8.-9. (canceled)

10. The method of claim 1, wherein the selection of the region is at least in part informed by the comparative analysis of the physical map and a reference.

11.-13. (canceled)

14. The method of claim 1, wherein the manipulation involves the delivery of at least one reagent in proximity to the region of said macromolecule, such that the at least one reagent can directly, or indirectly, enable, enhance, activate, or modify a reaction, binding, or cleaving within the region.

15.-28. (canceled)

29. The method of claim 1, wherein the physical or chemical manipulation involves the delivery of at least one photon in proximity to the region of said macromolecule, such that the at least one photon can directly, or indirectly, enable, enhance, activate, or modify a reaction, binding, or cleaving event within the region.

30.-35. (canceled)

36. The method of claim 1, wherein the physical or chemical manipulation involves the delivery of at least one contact probe in proximity to the region of said macromolecule, such that the at least one contact probe can directly, or indirectly, enable, enhance, activate, or modify a reaction, binding, or cleaving event within the region.

37.-41. (canceled)

42. The method of claim 1, wherein the physical or chemical manipulation involves the delivery of at least one drop of solution in proximity to the region of said macromolecule, such that the at least one drop of solution can directly, or indirectly, enable, enhance, activate, or modify a reaction, binding, or cleaving event within the region.

43.-44. (canceled)

45. The method of claim 1, wherein the macromolecule comprises a long nucleic acid molecule.

46. The method of claim 1, wherein the macromolecule is not cleaved prior to the physical or chemical manipulation.

47. (canceled)

48. The method of claim 1, wherein the macromolecule extracted from a sample retain at least some native three dimensional configuration.

49. The method of claim 1, wherein extracting comprises removing the individual macromolecule from the biological sample while retaining at least some binding moieties bound to the individual macromolecule.

50. The method of claim 1, wherein isolating comprises positioning the macromolecule such that at least a portion of the region is elongated in a fluidic device.

51. The method of claim 1, wherein isolating comprises positioning the macromolecule in a fluidic device such that it may be individually identified.

52. The method of claim 1, wherein isolating comprises positioning the macromolecule such that it may be individually manipulated in a fluidic device.

53. The method of claim 1, wherein the macromolecule is interrogated in a fluidic device.

54.-60. (canceled)
